(19) United States
(12) Patent Application Publication
(10) Pub. No.: US 2021/0261583 A1
(43) Pub. Date: Aug. 26, 2021

(54) Synthetic Cytotoxic Molecules, Drugs, Methods of Their Synthesis and Methods of Treatment

(71) Applicants: The Regents of the University of California, Oakland, CA (US); Universite de Montreal, Montreal (CA)

(72) Inventors: Aimee Edinger, Irvine, CA (US); Stephen Hanessian, Irvine, CA (US)

(73) Assignees: The Regents of the University of California, Oakland, CA (US); Universite de Montreal, Montreal, QC (CA)

(21) Appl. No.: 17/252,226

(22) PCT Filed: Jun. 14, 2019
(86) PCT No.: PCT/US2019/037362

§ 371 Date: Dec. 14, 2020

Related U.S. Application Data
(60) Provisional application No. 62/685,197, filed on Jun. 14, 2018.

(51) Int. Cl. C07F 9/572; C07D 207/12; C07D 487/04; A61K 45/06

(52) U.S. Cl. CPC C07F 9/572; A61K 45/06; C07D 487/04; C07D 207/12

(57) ABSTRACT

Small molecules compounds and methods of their synthesis are provided. Formulations and medicaments are also provided that are directed to the treatment of disease, such as, for example, neoplasms, cancers, and other diseases. Therapeutics are also provided containing a therapeutically effective dose of one or more small molecule compounds, present either as pharmaceutically effective salt or in pure form, including, but not limited to, formulations for oral, intravenous, or intramuscular administration.

### CROSS REFERENCE TO RELATED APPLICATIONS

This application claims priority to U.S. Provisional Application Ser. No. 62/685,197 entitled “Synthetic Cytotoxic Molecules, Drugs, Methods of Their Synthesis and Methods of Treatment,” filed Jun. 14, 2018, which is herein incorporated by reference in its entirety.

### STATEMENT REGARDING FEDERALLY SPONSORED RESEARCH OR DEVELOPMENT

This invention was made with Governmental support under Grant Nos. R01 GM089919 awarded by the National Institutes of Health. The government has certain rights in the invention.

### FIELD OF THE INVENTION

The invention is generally directed to synthetic cytotoxic molecules, medicaments formed from these molecules, methods of synthesis of these molecules, and methods for the treatment of disorders or neoplasms using such therapeutics.

### BACKGROUND

Sphingolipids are a class of molecules that are derivatives of sphingosine. These molecules are typically found in the membranes of cells and can trigger many different signaling cascades. In yeast, phytosphingosine is produced in response to environmental stress, triggering proliferative arrest by reducing surface levels of transporters for amino acids and uracil. The chemical structure of phytosphingosine is provided in FIG. 1. Phytosphingosine also triggers nutrient transporter down-regulation in mammalian cells.

Various compounds based on diastereomeric 3- and 4-C-aryl 2-hydroxymethyl pyrrolidines have been found to phenocopy the actions of phytosphingosine, disrupting nutrient transport systems and lysosomal fusion reactions, selectively killing cancer cells by limiting their access to nutrients. An example of these molecules, SH-BC-893, is illustrated in FIG. 1. These anti-cancer effects of SH-BC-893 and related synthetic sphingolipids occur independent of compound phosphorylation and sphingosine-1-phosphate receptor engagement.

### SUMMARY OF THE INVENTION

In many embodiments the invention is directed to small molecules, methods of synthesis, medicaments formed from these small molecules, and methods for the treatment of disorders using such therapeutics are disclosed.

In an embodiment is a compound of formula

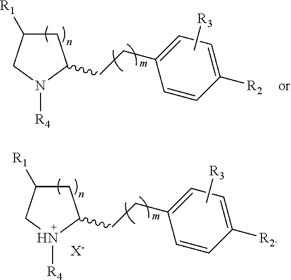

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne. R₂ is an aliphatic chain (C₆-C₁₄). R₃ is a mono-, di-, tri- or tetra-aromatic substituent that includes hydrogen, halogen, alkyl, alkoxy, azide (N₃), ether, NO₂, cyanide (CN), or a combination thereof. R₄ is a functional group selected from H, alkyl including methyl (Me), Boc, or Ac. X⁻ is an anion of the suitable acid. n is an independently selected integer selected from 1, 2, or 3. m is an independently selected integer selected from 0, 1 or 2. The compound also includes an optional functional group of the azacycle's substituent selected from the following: a polar group in the alpha, beta or gamma position with regard to the azacycle selected from carbonyls (C═O) and alcohols (CHOH); a cyclic carbon chain extending from the alpha, beta or gamma positions with regard to the azacycle back to the N of the azacycle; and a combination thereof.

In another embodiment, the compound is selected from:

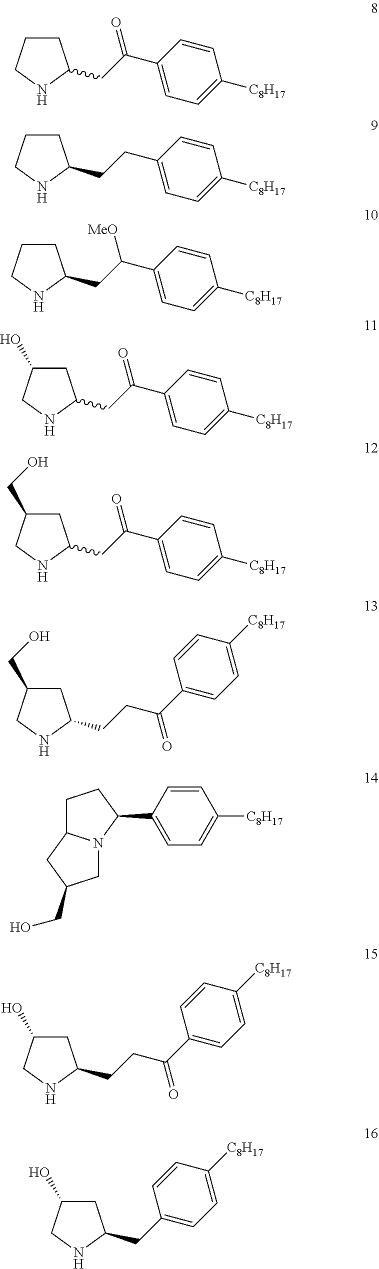

In yet another embodiment, the compound is:

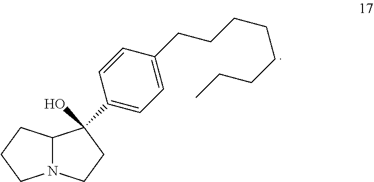 [SMILES: CCCCCCCCc1ccc([C@]2(O)CCN3CCCC32)cc1]

In a further embodiment, the compound is selected from:

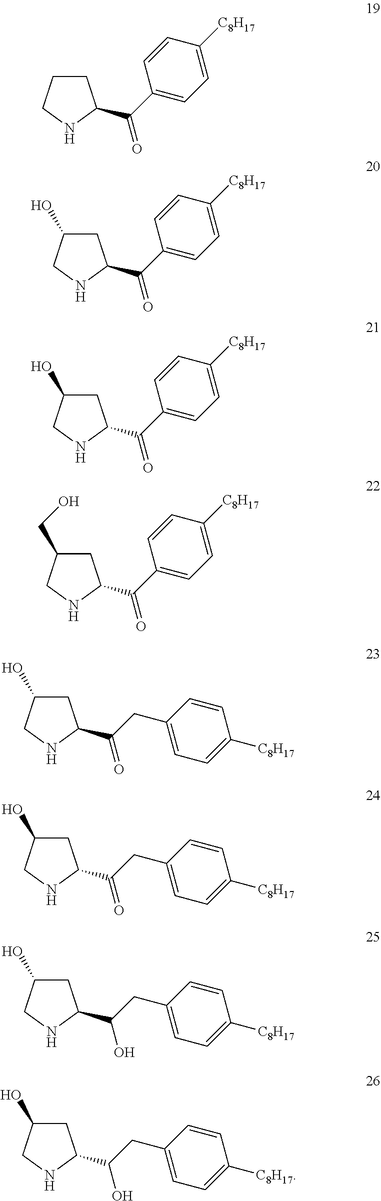

In still yet another embodiment, the compound is selected from:

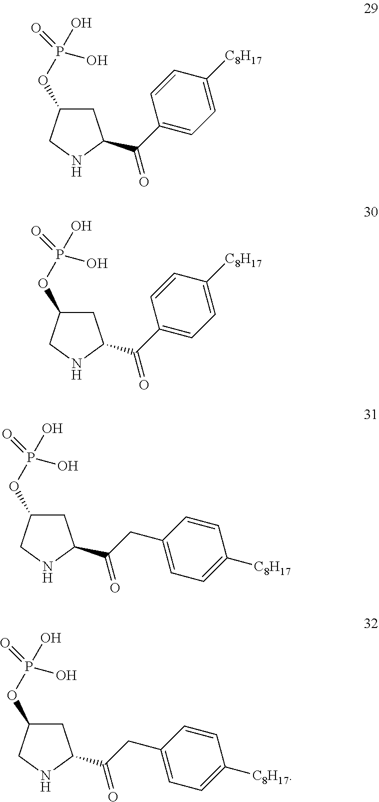

In yet a further embodiment, the compound is capable of having a cytotoxic effect on human neoplastic cells, and wherein the cytotoxic effect is defined by a reduction in the percentage of viable human neoplastic cells.

In an even further embodiment, the cytotoxic effect is achieved with a local 50% inhibitory concentration (IC₅₀) of less than twenty micromolar, wherein the local IC₅₀ is defined by the concentration of the compound that reduces the percentage of viable human neoplastic cells by 50%.

In yet an even further embodiment, the human neoplastic cells are derived from at least one neoplasm. The at least one neoplasm is selected from: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

In still yet an even further embodiment, the human neoplastic cells are characterized by one of: fast-growing, aggressive, Warburg-phenotypic, malignant, Ras-positive, PTEN-negative, having PI 3-kinase mutations, benign, metastatic, or nodular.

In still yet an even further embodiment, the compound is capable of exerting bioenergetic stress on human cells. The bioenergetic stress is characterized by a decrease of at least one nutrient available to the human cells, and wherein the at least one nutrient is selected from one or more of the group: glucose, amino acids, nucleotides, and lipids.

In still yet an even further embodiment, the human cells are comprised of neoplastic and non-neoplastic cells. The bioenergetic stress results in greater percentage of cell death in the neoplastic cells relative to non-neoplastic cells.

In still yet an even further embodiment, the compound is capable of inhibiting growth of a tumor comprised of human neoplastic cells. Growth is defined by at least one growth assessment. The at least one growth assessment is selected from: an increase in tumor diameter, an increase in tumor bioluminescence, an increase in tumor volume, an increase in tumor mass, or an increase in neoplastic cell proliferation rate.

In an embodiment, a medicament for the treatment of a human disorder includes a pharmaceutical formulation containing a therapeutically effective amount of one or more small molecule compounds having the formula

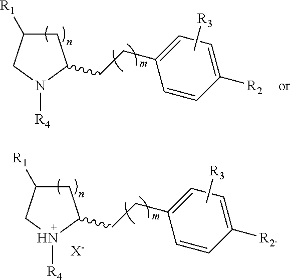

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne. R₂ is an aliphatic chain (C₆-C₁₄). R₃ is a mono-, di-, tri- or tetra-aromatic substituent that includes hydrogen, halogen, alkyl, alkoxy, azide (N₃), ether, NO₂, cyanide (CN), or a combination thereof. R₄ is a functional group selected from H, alkyl including methyl (Me), Boc, or Ac. X⁻ is an anion of the suitable acid. n is an independently selected integer selected from 1, 2, or 3. m is an independently selected integer selected from 0, 1 or 2. The compound also includes an optional functional group of the azacycle's substituent selected from the following: a polar group in the alpha, beta or gamma position with regard to the azacycle selected from carbonyls (C═O) and alcohols (CHOH); a cyclic carbon chain extending from the alpha, beta or gamma positions with regard to the azacycle back to the N of the azacycle; and a combination thereof.

In another embodiment, the compound is selected from:

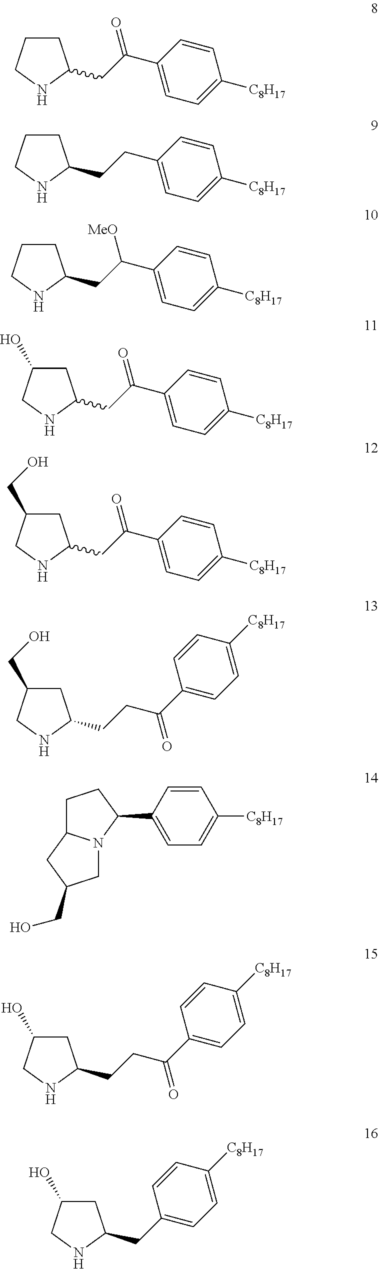

In yet another embodiment, the compound is:

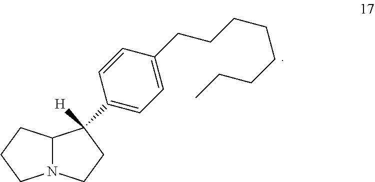 [SMILES: CCCCCCCCc1ccc([C@@H]2CCN3CCCC23)cc1]

In a further embodiment, the compound is selected from:

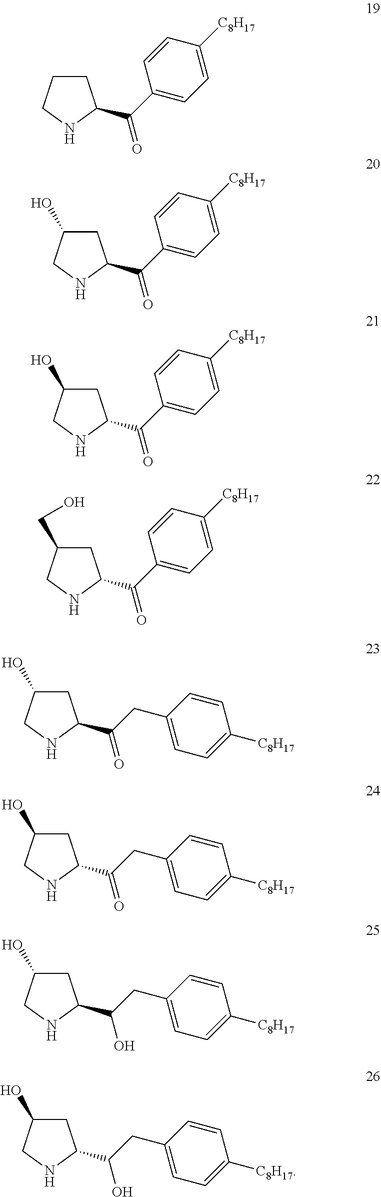

In still yet another embodiment, the compound is selected from:

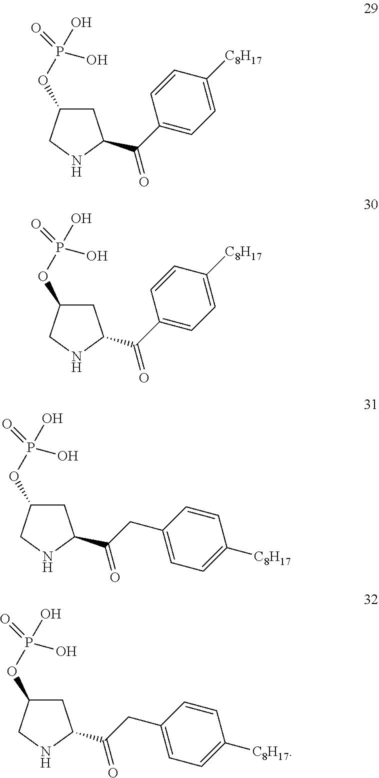

In yet a further embodiment, the compound is capable of having a cytotoxic effect on human neoplastic cells, and wherein the cytotoxic effect is defined by a reduction in the percentage of viable human neoplastic cells.

In an even further embodiment, the cytotoxic effect is achieved with a local 50% inhibitory concentration (IC₅₀) of less than twenty micromolar, wherein the local IC₅₀ is defined by the concentration of the compound that reduces the percentage of viable human neoplastic cells by 50%.

In yet an even further embodiment, the human neoplastic cells are derived from at least one neoplasm. The at least one neoplasm is selected from: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

In still yet an even further embodiment, the human neoplastic cells are characterized by one of: fast-growing, aggressive, Warburg-phenotypic, malignant, Ras-positive, PTEN-negative, having PI 3-kinase mutations, benign, metastatic, or nodular.

In still yet an even further embodiment, the compound is capable of exerting bioenergetic stress on human cells. The bioenergetic stress is characterized by a decrease of at least one nutrient available to the human cells, and wherein the at least one nutrient is selected from one or more of the group: glucose, amino acids, nucleotides, and lipids.

In still yet an even further embodiment, the human cells are comprised of neoplastic and non-neoplastic cells. The bioenergetic stress results in greater percentage of cell death in the neoplastic cells relative to non-neoplastic cells.

In still yet an even further embodiment, the compound is capable of inhibiting growth of a tumor comprised of human neoplastic cells. Growth is defined by at least one growth assessment. The at least one growth assessment is selected from: an increase in tumor diameter, an increase in tumor bioluminescence, an increase in tumor volume, an increase in tumor mass, or an increase in neoplastic cell proliferation rate.

In still yet an even further embodiment, the medicament further includes at least one cytotoxic FDA-approved compound for the treatment of a neoplasm.

In still yet an even further embodiment, the at least one cytotoxic FDA-approved compound is selected from the group: methotrexate, gemcitabine, tamoxifen, taxol, docetaxel, and enzalutamide.

In an embodiment, a method for treatment of a human disorder includes administering a pharmaceutical formulation to a human subject, the pharmaceutical formulation containing a therapeutically effective amount of one or more small molecule compounds having the formula

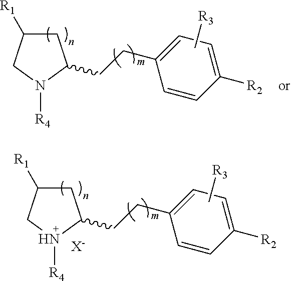

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne. R₂ is an aliphatic chain (C₆-C₁₄). R₃ is a mono-, di-, tri- or tetra-aromatic substituent that includes hydrogen, halogen, alkyl, alkoxy, azide (N₃), ether, NO₂, cyanide (CN), or a combination thereof. R₄ is a functional group selected from H, alkyl including methyl (Me), Boc, or Ac. X⁻ is an anion of the suitable acid. n is an independently selected integer selected from 1, 2, or 3. m is an independently selected integer selected from 0, 1 or 2. The compound also includes an optional functional group of the azacycle's substituent selected from the following: a polar group in the alpha, beta or gamma position with regard to the azacycle selected from carbonyls (C═O) and alcohols (CHOH); a cyclic carbon chain extending from the alpha, beta or gamma positions with regard to the azacycle back to the N of the azacycle; and a combination thereof.

In another embodiment, the compound is selected from:

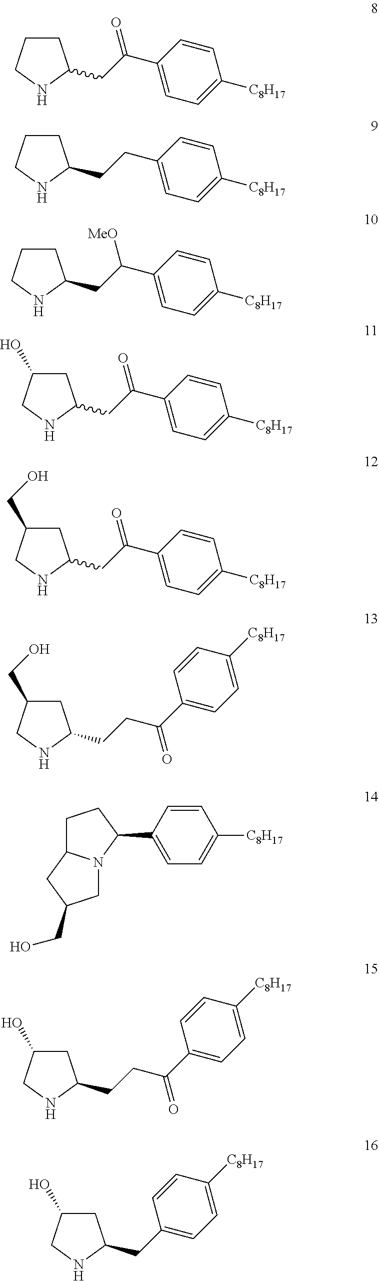

In yet another embodiment, the compound is:

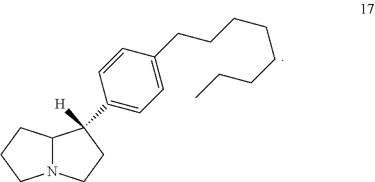 [SMILES: CCCCCCCCc1ccc([C@@H]2CCN3CCCC23)cc1]

In a further embodiment, the compound is selected from:

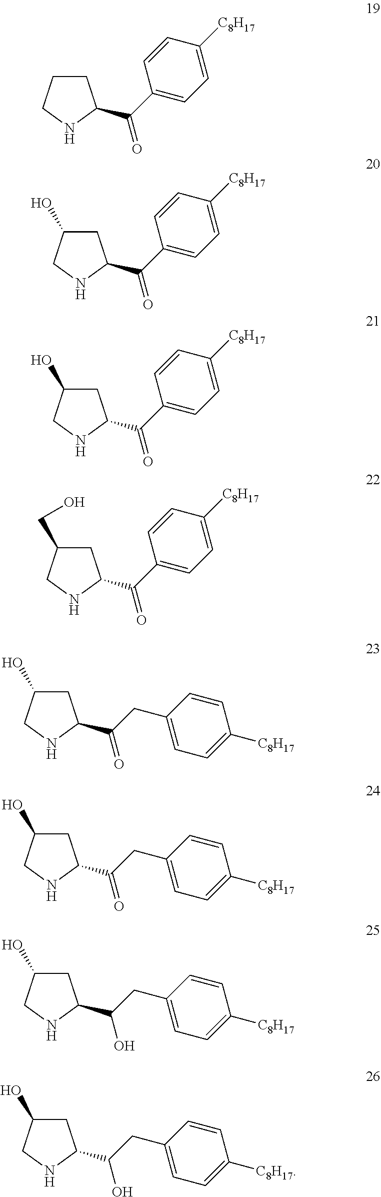

In still yet another embodiment, the compound is selected from:

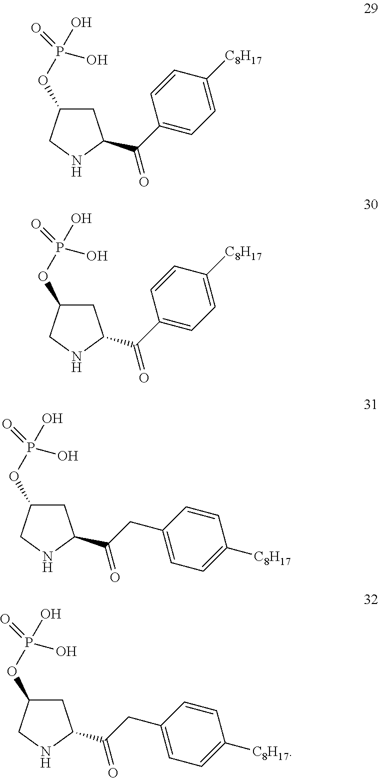

In yet a further embodiment, the method further includes diagnosing the human subject with at least one human disorder.

In an even further embodiment, the at least one human disorder is a neoplasm. The neoplasm is selected from one or more of the group: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

In yet an even further embodiment, the pharmaceutical formulation inhibits growth of a tumor comprising human neoplastic cells. Growth is defined by at least one growth assessment. The at least one growth assessment is selected from one or more of the group: an increase in tumor diameter, an increase in tumor bioluminescence, an increase in tumor volume, an increase in tumor mass, and an increase in neoplastic cell proliferation rate.

In still yet an even further embodiment, the human disorder is characterized by at least one neoplasm characterization. The at least one neoplasm characterization is selected from one or more of the group: fast-growing, aggressive, Warburg-phenotypic, malignant, Ras-positive, PTEN-negative, having PI 3-kinase mutations, benign, metastatic, or nodular.

In still yet an even further embodiment, the treatment is combined with an FDA-approved standard of care.

In still yet an even further embodiment, the pharmaceutical formulation is combined with at least one cytotoxic FDA-approved compound.

In still yet an even further embodiment, the at least one cytotoxic FDA-approved compound is selected from: methotrexate, gemcitabine, tamoxifen, taxol, docetaxel, and enzalutam ide.

In an embodiment is a compound having the formula

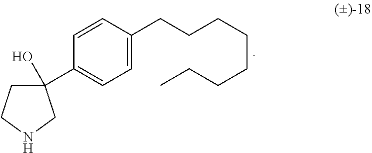 [SMILES: CCCCCCCCc1ccc(C2(O)CCNC2)cc1]

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a molecular structure of phytosphingosine and SH-BC-893 in accordance with the prior art.

FIG. 2 provides a molecular structure diagram of a number of therapeutic small molecule analogs in accordance with various embodiments of the invention.

FIG. 3 provide examples of molecular structures of therapeutic small molecule analogs in accordance with various embodiments of the invention.

FIGS. 4 to 7 provide reaction pathways for the production of therapeutic small molecule analogs in accordance with various embodiments of the invention.

FIGS. 8 to 9 provide examples of molecular structures of therapeutic small molecule analogs in accordance with various embodiments of the invention.

FIG. 10 provides reaction pathways for the production of therapeutic small molecule analogs in accordance with various embodiments of the invention.

FIG. 11 provides examples of molecular structures of therapeutic small molecule analogs in accordance with various embodiments of the invention.

### DETAILED DESCRIPTION

Turning now to the drawings and data, molecules capable of treating disorders, including neoplasms and cancer, from a variety of therapeutic mechanisms including triggering cellular nutrient transporter down-regulation and blocking lysosomal fusion reactions, medicaments formed from these molecules, methods of synthesis of these molecules, and methods for the treatment of disorders using such therapeutics are disclosed. In some embodiments, the molecules are 2-C-aryl azacycle molecules. In some embodiments, the molecules are 2-C-aryl pyrrolidines. In some embodiments, the molecules are pharmaceutically acceptable salts of 2-C-aryl azacycle molecules. In other embodiments, formulations and medicaments are provided that are directed to the treatment of disorders. In some such embodiments these formulations and medicaments target cancers, such as, for example, leukemia, prostate, colon, lung, pancreatic and breast cancer, and potentially other disorders, including metabolic disorders or disorders where oncogenic Ras or PI 3-kinase mutations or PTEN loss are associated with the neoplastic cells. Therapeutic embodiments contain a therapeutically effective dose of one or more small molecule compounds. Embodiments allow for various formulations, including, but not limited to, formulations for oral, intravenous, or intramuscular administration. Other additional embodiments provide treatment regimens for disorders using therapeutic amounts of the small molecules.

In addition to embodiments of medicaments and treatments, embodiments are directed to the ability of 2-C-aryl azacycle molecules to induce changes in cellular bioenergetics in cells. Embodiments of the mechanism will induce bioenergetic stress due to a decrease in access to nutrients. Accordingly, in some embodiments, the stress will cause death of neoplastic cells while not causing toxicity in normal, healthy cells. Many embodiments of the invention are directed to the ability of these molecules to decrease nutrient transporters on a cell surface, low-density lipoprotein degradation, macropinosome degradation, and autophagy.

### Terms of Art

“Acyl” means a —C(═O)R group.

“Alcohol” means a hydrocarbon with an —OH group (ROH).

“Alkyl” refers to the partial structure that remains when a hydrogen atom is removed from an alkane.

“Alkyl phosphonate” means an acyl group bonded to a phosphate, RCO₂PO₃².

“Alka ne” means a compound of carbon and hydrogen that contains only single bonds.

“Alkene” refers to an unsaturated hydrocarbon that contains at least one carbon-carbon double bond.

“Alkyne” refers to an unsaturated hydrocarbon that contains at least one carbon-carbon triple bond.

“Alkoxy” refers to a portion of a molecular structure featuring an alkyl group bonded to an oxygen atom.

“Aryl” refers to any functional group or substituent derived from an aromatic ring.

“Amine” molecules are compounds containing one or more organic substituents bonded to a nitrogen atom, RNH₂, R₂NH, or R₃N.

“Amino acid” refers to a difunctional compound with an amino group on the carbon atom next to the carboxyl group, RCH(NH₂)CO₂H.

“Azide” refers to N₃.

“Cyanide” refers to CN.

“Ester” is a compound containing the —CO₂R functional group.

“Ether” refers to a compound that has two organic substituents bonded to the same oxygen atom, i.e., R—O—R′.

“Halogen” or “halo” means fluoro (F), chloro (Cl), bromo (Br), or iodo (I).

“Hydrocarbon” means an organic chemical compound that consists entirely of the elements carbon (C) and hydrogen (H).

“Phosphate”, “phosphonate”, or “PO” means a compound containing the elements phosphorous (P) and oxygen (O).

“R” in the molecular formulas above and throughout are meant to indicate any suitable organic functionality.

### 2-C-aryl Azacycle Molecules

Compounds in accordance with embodiments of the invention are based on diastereomeric 2-C-aryl azacycles. A chemical compound in accordance with embodiments of the invention is illustrated in FIG. 2 and pictured below. Embodiments comprise the molecules as illustrated in FIG. 2, including an azacycle compound and its salt of a suitable acid:

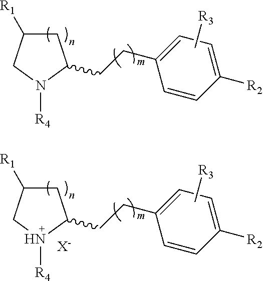

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne.

R₂ is an aliphatic chain (C₆-C₁₄). R₃ is a mono-, di-, tri- or tetra-aromatic substituent that includes hydrogen, halogen, alkyl, alkoxy, azide (N₃), ether, NO₂, cyanide (CN), or a combination thereof.

R₄ is a functional group selected from H, alkyl including methyl (Me), ester, or acyl.

X⁻ is an anion of the suitable acid.

n is an independently selected integer selected from 1, 2, or 3.

m is an independently selected integer selected from 0, 1 or 2; and comprising.

The molecule can include an optional functional group of the azacycle's substituent selected from the following:

a polar group in the alpha, beta or gamma position with regard to the azacycle selected from carbonyls (C═O) and alcohols (CHOH);

*a cyclic carbon chain extending from the alpha, beta or gamma positions with regard to the azacycle back to the N of the azacycle, and

a combination thereof.

In some embodiments, R₁ is H, OH, CH₂OH, OPO(OH)_{2.} In some embodiments, R₁ is H. In some embodiments, R₁ is OH. In some embodiments, R₁ is CH₂OH. In some embodiments, R₁ is OPO(OH)₂.

In some embodiments, R₂ is C₆₋₁₄ alkyl, C₆₋₁₀ alkyl, C₇₋₉ alkyl, C₆H₁₃, C₇H₁₅, C₈H₁₇, C₉H₁₉, C₁₀H₂₁, C₁₁H₂₃, C₁₂H₂₅, C₁₃H₂₇, or C₁₄H₂₉. In some embodiments, R₂ is C₈H₁₇.

In some embodiments R₃ is H.

In some embodiments, n is 1.

In some embodiments m is 0. In some embodiments, m is 1. In some embodiments, m is 2. In some embodiments, m is 3.

In some embodiments, the linking group connecting the phenyl ring to the azacycle is C(═O), CH₂C(═O), C(═O)CH₂, CH₂CH₂C(═O), CH_{2,}CH₂CH₂, CH₂C(OCH₃)H, or CHOHCH₂. In some embodiments, the linking group connecting the phenyl ring to the azacycle is C(═O). In some embodiments, the linking group connecting the phenyl ring to the azacycle is CH₂C(═O). In some embodiments, the linking group connecting the phenyl ring to the azacycle is C(═O)CH₂. In some embodiments, the linking group connecting the phenyl ring to the azacycle is CH₂CH₂C(═O). In some embodiments, the linking group connecting the phenyl ring to the azacycle is CH₂. In some embodiments, the linking group connecting the phenyl ring to the azacycle is CH₂CH₂. In some embodiments, the linking group connecting the phenyl ring to the azacycle is CH₂C(OCH₃)H. In some embodiments, the linking group connecting the phenyl ring to the azacycle is CHOHCH₂.

In some embodiments, the linking group connecting the phenyl ring to the azacycle includes a cyclic carbon chain extending from the alpha, beta or gamma positions with regard to the azacycle back to the N of the azacycle, so that the azacycle with the linking group form an optionally substituted bicyclic ring of the formula:

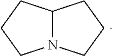

In some embodiments, R₄ is H. In some embodiments, R₄ is C₁₋₆ alkyl, such as CH₃, C₂H₅, C₃H₇, C₄H₉, C₅H₁₁, C₆H₁₃, C₁₋₃ alkyl, etc., C₁₋₆ acyl, or C₁₋₆ ester. In some embodiments, R₄ is methyl.

In still other embodiments, the R₂ and R₃ substituents can have different combinations around the phenyl ring with regard to their position.

In still other embodiments, R₂ is an unsaturated hydrocarbon chain.

In still other embodiments, the R₁ is an alkyl having 1 to 6 carbons.

It will be understood that compounds in this invention may exist as stereoisomers, including enantiomers, diastereomers, cis, trans, syn, anti, solvates (including hydrates), tautomers, and mixtures thereof, are contemplated in the compounds of the present invention.

The claimed inventions can also be related to pharmaceutically acceptable salts. A “pharmaceutically acceptable salt” retains the desirable biological activity of the compound without undesired toxicological effects. Salts can be salts with a suitable acid, including, but not limited to, hydrochloric acid, hydrobromic acid, sulfuric acid, phosphoric acid, nitric acid and the like; acetic acid, oxalic acid, tartaric acid, succinic acid, malic acid, benzoic acid, pamoic acid, alginic acid, methanesulfonic acid, naphthalenesulphonic acid, and the like. Also, incorporated cations can include ammonium, sodium, potassium, lithium, zinc, copper, barium, bismuth, calcium, and the like; or organic cations such as tetraalkylammonium and trialkylammonium cations. Also useful are combinations of acidic and cationic salts. Included are salts of other acids and/or cations, such as salts with trifluoroacetic acid, chloroacetic acid, and trichloroacetic acid.

Other azacyclic sphingolipid-like molecules, as well as modified azacyclic sphingolipid-like molecules, suitable for practice of the present invention will be apparent to the skilled practitioner. Some molecules may include any diastereomeric C-aryl pyrrolidine compound. Furthermore, these molecules may employ several mechanisms of action to inhibit neoplasm growth, without inducing toxic S1P receptor activity, even if the molecules are not structurally identical to the compounds shown above.

### Modes of Treatment

In some embodiments, the azacyclic sphingolipid-like compounds are administered in a therapeutically effective amount as part of a course of treatment. As used in this context, to “treat” means to ameliorate at least one symptom of the disorder to be treated or to provide a beneficial physiological effect. For example, one such amelioration of a symptom could be inhibition of neoplastic proliferation. Assessment of neoplastic proliferation can be performed in many ways, including, but not limited to assessing changes in tumor diameter, changes in tumor bioluminescence, changes in tumor volume, changes in tumor mass, or changes in neoplastic cell proliferation rate.

In several embodiments, an individual to be treated has been diagnosed as having a neoplastic growth or cancer. In many embodiments, the neoplasm is characterized as fast-growing, aggressive, Warburg-phenotypic, malignant, Ras-positive, PTEN-negative, having PI 3-kinase mutations, benign, metastatic, or nodular. A number of cancers can be treated, including (but not limited to) acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, or vascular tumors.

A therapeutically effective amount can be an amount sufficient to prevent reduce, ameliorate or eliminate the symptoms of diseases or pathological conditions susceptible to such treatment, such as, for example, cancers like leukemia, prostate, colon, lung, pancreatic, or breast cancer, or diseases where oncogenic Ras mutations afford multiple metabolic advantages to transformed cells. In some embodiments, a therapeutically effective amount is an amount sufficient to reduce the transport of nutrients, such as, for example, glucose, amino acids, nucleotides or lipids, into cells.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to non-neoplastic cells and, thereby, reduce side effects.

Data obtained from cell culture assays or animal studies can be used in formulating a range of dosage for use in humans. If the medicament is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of neoplastic growth) as determined in cell culture. Such information can be used to more accurately determine useful doses in humans. Levels in plasma may be measured, for example, by liquid chromatography coupled to mass spectrometry. In some embodiments, a cytotoxic effect is achieved with an IC₅₀ less than 100 μM, 50 μM, 20 μM, 10 μM, or 5 μM.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result. A single azacyclic sphingolipid-like small molecule compound may be administered, or combinations of various azacyclic sphingolipid-like small molecule compounds may also be administered.

In a number of embodiments, azacyclic sphingolipid-like small molecule compounds are administered in combination with an appropriate standard of care, such as the standard of care established by the United States Federal Drug Administration (FDA). In many embodiments, azacyclic sphingolipid-like small molecule compounds are administered in combination with other cytotoxic compounds, especially FDA-approved compounds. A number of FDA-approved cytotoxic compounds can be utilized, including (but not limited to) methotrexate, gemcitabine, tamoxifen, taxol, docetaxel, and enzalutam ide.

It is also possible to add agents that improve the solubility of these compounds. For example, the claimed compounds can be formulated with one or more adjuvants and/or pharmaceutically acceptable carriers according to the selected route of administration. For oral applications, gelatin, flavoring agents, or coating material can be added. In general, for solutions or emulsions, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

Numerous coating agents can be used in accordance with various embodiments of the invention. In some embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

Various embodiments of formulations also include at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

Several embodiments of a formulation further utilize other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Multiple embodiments of a formulation also include a surfactant. In certain embodiments, surfactants are selected from the group consisting of Tween 80, sodium lauryl sulfate, and docusate sodium.

Many embodiments of a formulation further utilize a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

Various embodiments of a formulation also include a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Modes of administration, in accordance with multiple embodiments, include, but are not limited to, oral, transdermal, transmucosal (e.g., sublingual, nasal, vaginal or rectal), or parenteral (e.g., subcutaneous, intramuscular, intravenous, bolus or continuous infusion). The actual amount of drug needed will depend on factors such as the size, age and severity of disease in the afflicted individual. The actual amount of drug needed will also depend on the effective concentration ranges of the various active ingredients.

A number of embodiments of formulations include those suitable for oral administration. Formulations may conveniently be presented in unit dosage form and may be prepared by any of the methods well known in the art of pharmacy. Typically, these methods include the step of bringing into association a compound of at least one embodiment described herein, or a pharmaceutically salt, prodrug, or solvate thereof (“active ingredient”) with the carrier which constitutes one or more accessory ingredients.

Embodiments of formulations disclosed herein suitable for oral administration may be presented as discrete units such as capsules, cachets or tablets each containing a predetermined amount of the active ingredient; as a powder or granules; as a solution or a suspension in an aqueous liquid or a nonaqueous liquid; or as an oil-in-water liquid emulsion or a water-in-oil liquid emulsion. Multiple embodiments also compartmentalize various components within a capsule, cachets, or tablets, or any other appropriate distribution technique.

Several embodiments of pharmaceutical preparations include tablets, push-fit capsules made of gelatin, as well as soft, sealed capsules made of gelatin and a plasticizer, such as glycerol or sorbitol. Tablets, in a number of embodiments, may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. Push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### Exemplary Embodiments

Biological data supports the use of the aforementioned azacyclic sphingolipid-like compounds in a variety of embodiments to treat disease. It is noted that embodiments of azacyclic 2-C-aryl analogs of FTY720, in accordance with the disclosure, kill and/or inhibit the growth of neoplastic cells. Accordingly, embodiments using these compounds to treat various diseases, such as cancer, avoid the pitfalls associated with prior approaches.

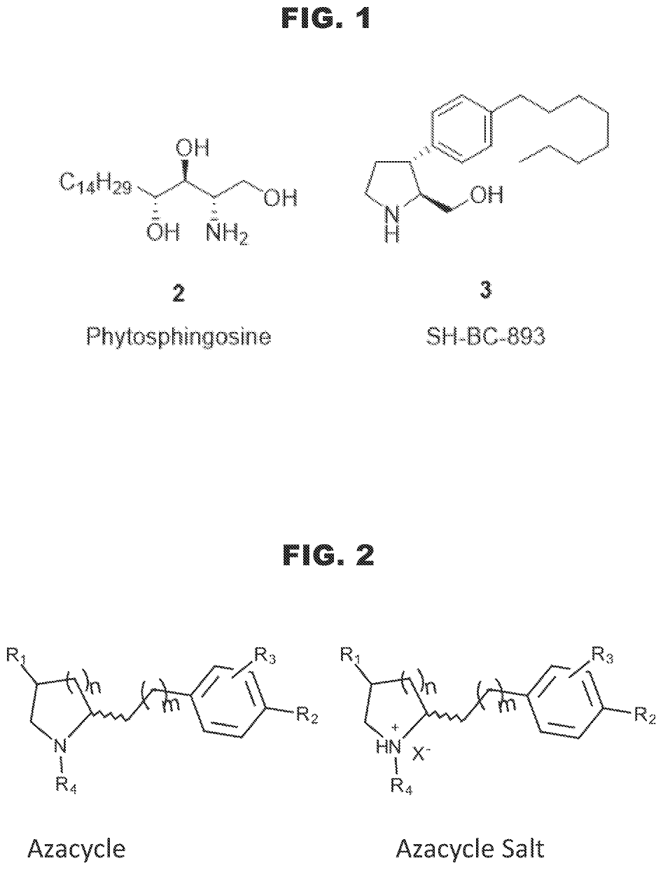

Cancer chemotherapy remains an enigmatic and challenging endeavor. In spite of heroic efforts and impressive advances on many fronts, major obstacles such as resistance and toxicity plague the search for effective drugs. Compounds that exploit the metabolic differences between cancer and normal cells provide an alternative to toxic systemic chemotherapies or therapies targeting oncogenic signal transduction cascades. In prior studies, it was shown that natural phytosphingosine (2) (FIG. 1) kills cancer cells by interfering with one or more nutrient transport systems required for sustenance (V. Brinkman, et al., Nat. Rev. Drug. Discov. 9 (2010) 883-897; L Zhang, et al., Oncology Reports 30 (2013) 2571-2578; and R. Fransson, et al., ACS Med. Chem. Lett. 4, (2013), 969-973; the disclosures of which are herein incorporated by reference). This strategy of “starving cancer cells to death” has been effectively demonstrated in vitro and in vivo with the analog SH-BC-893 (3), which is not phosphorylated in vivo and avoids cardiovascular effects induced by interaction with sphingosine-1-phosphate receptors (S. M. Kim, et al., J. Clin. Invest. 126 (2016) 4088-4102; and M. S. Perryman, et al., Bioorg. Med. Chem. 24 (2016) 4390-4397; the disclosures of which are herein incorporated by reference). The analog SH-BC-893 and other similar analogs are described in the U.S. Patent Application No. 15/760,199, the disclosure of which is herein incorporated by reference.

It has been shown that the four nutrient uptake mechanisms used by mammalian cells, cell surface transporters for amino acids and glucose, receptor-mediated LDL uptake and processing, autophagy, and macropinocytosis, are inhibited by analog 3 (S. M. Kim, et al., cited supra). Remarkably, cytotoxicity is limited to cancer cells, most likely because non-transformed cells can adapt to the stress caused by nutrient deprivation by altering their metabolic program. The ability of 3 to kill cancer cells at μM doses is attributed in part to protein phosphatase 2 (PP2A) activation which restricts access to nutrients by down-regulating amino acid and glucose transporters from the cell surface and blocking lysosomal fusion (S. M. Kim, et al., cited supra).

Variations of chain length, stereochemistry, and functional group manipulations were also performed to establish thresholds of activity for each of three phenotypes: viability, transporter loss, and vacuolation (M. S. Perryman, et al., cited supra). Various analogs with an aryloctyl chain repositioned to the 2-postion of the pyrrolidine framework were considered, many of which included polar substituents within geometric proximity to the pyrrolidine nitrogen. These analogs are discussed further in the examples described below.

### Extended C-2 Modified Analogs of 3

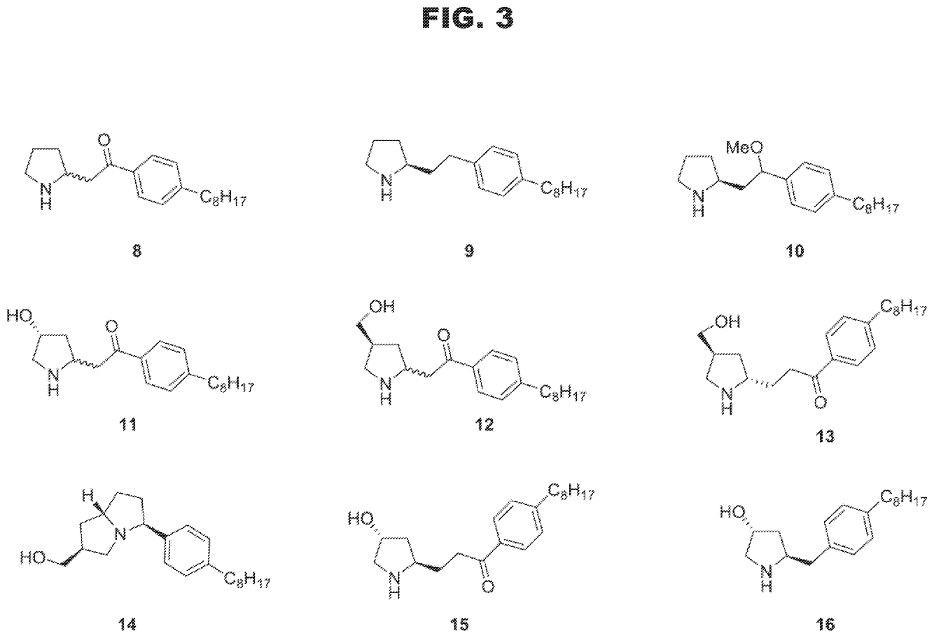

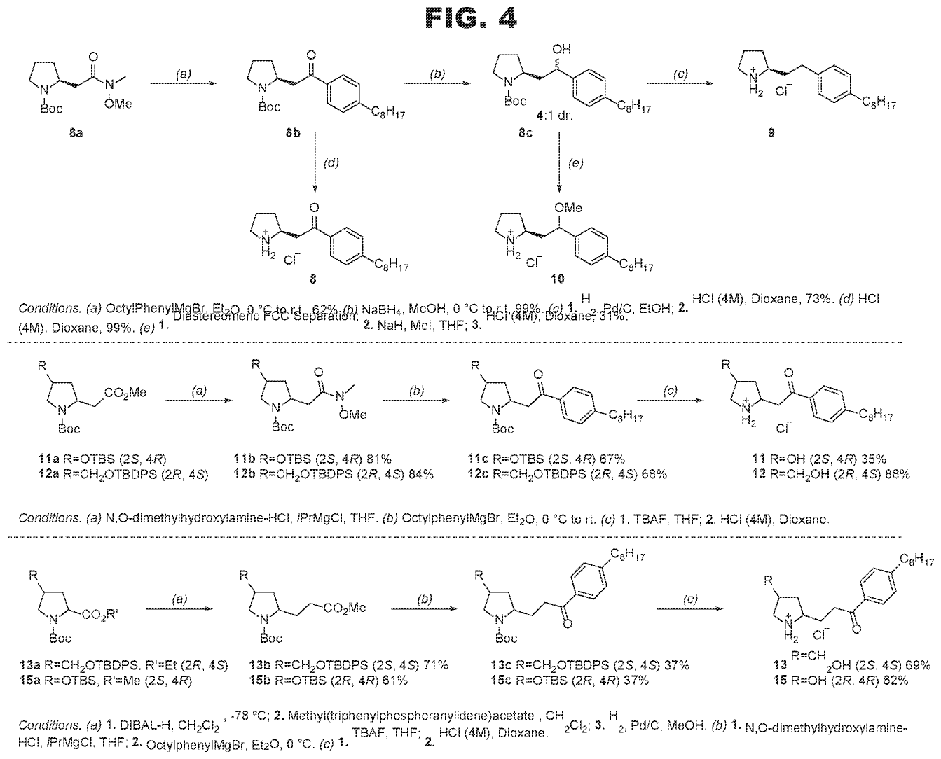

Being cognizant that the pyrrolidine core had to maintain its basic character for cytotoxicity (See R. Fransson, et al., cited supra; S. M. Kim, et al., cited supra; and B. Chen, et al., ACS Chem. Biol. 11 (2016) 409-414, the disclosure of which is herein incorporated by reference), the positioning of the aryloctyl side chain within the pyrrolidine ring was probed to determine if modified analogs would still maintain activity (FIG. 3). To this end, a series of 2-substituted pyrrolidines with extended chains was generated. To determine whether a polar moiety on the side chain affected activity, various analogs incorporated a ketone at the beta and gamma positions next to the pyrrolidine ring (Table 2). The analogs in this new series exhibited cytotoxicity similar to 3, down-regulating nutrient transporters and vacuolating at concentrations near their IC₅₀ and thus likely shared 3′s mechanism of action (Table 1).

TABLE 1
Cytotoxicity, nutrient down-regulation and vacuolation profiles of the C-2 modified analogs

| | | | % CD98 down-regulation | | | Vacuolation score | | |
|---|---|---|---|---|---|---|---|---|
| Comp. | IC50 (μM) [95% Cl] | | 2.5 μM | 10 μM | 40 μM | 2.5 μM | 10 μM | 40 μM |
| 3 | 2.1 | [2.0, 2.2] | 42 | 66 | n.d. | ++ | +++ | n.d. |
| 8 | 2.5 | [2.3, 2.8] | 78 | 57 | n.d. | + | +++ | n.d. |
| 9 | 2.8 | [2.6, 3.0] | 41 | 63 | n.d. | ++ | +++ | n.d. |
| 10 | 3.0 | [2.7, 3.3] | 15 | 58 | n.d. | 0 | +++ | n.d. |
| 11 | 2.1 | [1.5, 2.8] | 22 | 46 | n.d. | +++ | +++ | n.d. |
| 12 | 2.0 | [1.6, 2.6] | 44 | 50 | n.d. | ++ | +++ | n.d. |
| 13 | 4.0 | [3.5, 4.6] | 8 | 46 | n.d. | 0 | +++ | n.d. |
| 14 | 12.2 | [11.4, 13.0] | 0 | 32 | 68 | 0 | 0 | + |
| 15 | 2.1 | [1.9, 2.3] | 15 | 52 | n.d. | ++ | +++ | n.d. |
| 16 | 2.2 | [2.1, 2.3] | 40 | 63 | n.d. | ++ | +++ | n.d. |

Analogs 8, 9, and 10 were prepared from the Weinreb amide derivative of L-homoproline 8a previously reported by Georg et al. (F. S. Kimball, et al., Bioorg. Med. Chem. 16 (2008) 4367-4377, the disclosure of which is herein incorporated by reference). Treatment of 8a with octylphenylmagnesium bromide led to benzylic ketone 8b as a versatile common intermediate, using 3 equivalents of the Grignard reagent in Et₂O at 0° C. (FIG. 4). The presence of by-products from the reagent necessitated careful chromatography of the crude reaction product affording 62% yield of pure 8b. Removal of the N-Boc group with 4 N HCl in dioxane afforded 8 as a mixture of enantiomers due to rapid racemization in methanol or water (vide infra). Ketone 8b could also be reduced with NaBH₄ to give the corresponding benzylic alcohol 8c as a 4:1 diastereomeric mixture which could be easily separated by column chromatography. Although the stereochemistry of each diastereomer was not determined, the major diastereomer was converted to the methyl ether, then deprotected to afford product 10. The crude diastereomeric mixture of 8c was also catalytically hydrogenated at atmospheric pressure in ethanol, affording product 9 after final removal of the N-Boc protective group.

Products 11 and 12 were obtained starting from the 4-substituted homoprolines 11a and 12a respectively (FIG. 4) (see Synthesis and Characterization Section below). The corresponding Weinreb amides 11b and 12b were treated with octylphenylmagnesium bromide to give ketones 11c and 12c with acceptable yields. Careful chromatographic purification to separate by-products resulting from the Grignard reagent followed by treatment with TBAF and acid led to products 11 and 12. Products 13 and 15 were prepared starting from 13a (FIG. 4) (see Synthesis and Characterization Section) and commercially available 15a. Reduction of the ester group to the corresponding aldehyde with DIBAL-H, followed by a Wittig reaction with methyl(triphenylphosphoranylidene)acetate, and catalytic hydrogenation afforded the ester intermediates 13b an 15b. The corresponding Weinreb amides were subsequently reacted with octylphenylmagnesium bromide to give ketones 13c and 15c, each in 37% yield over two steps. Removal of the OTBDPS and N-Boc groups afforded products 13 and 15.

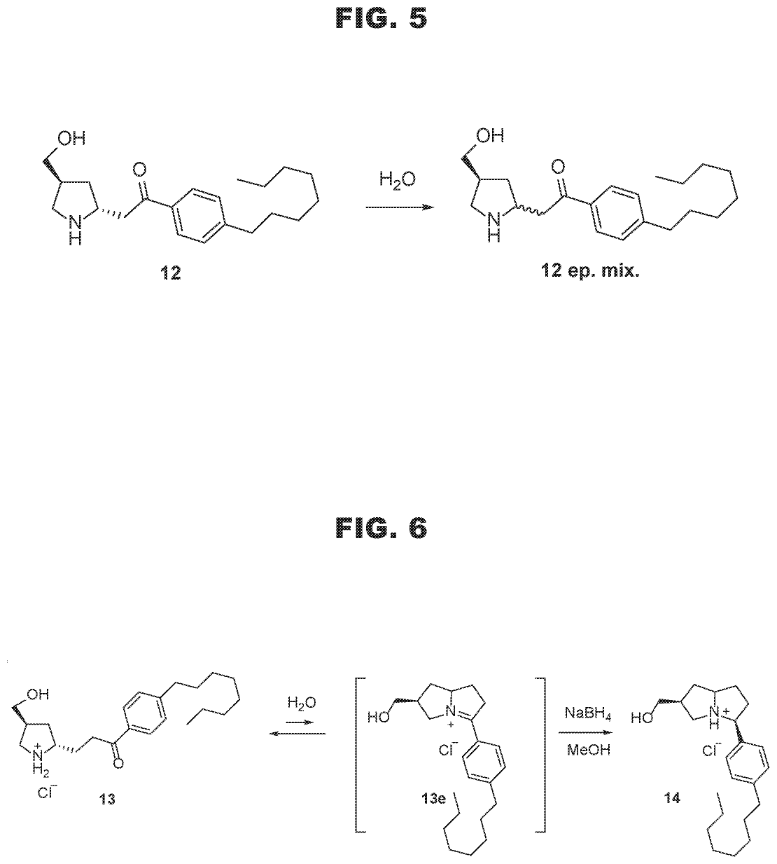

In the course of synthetic steps leading to analogs 8, 11 and 12, it was noted that after the deprotection of the N-Boc group the resulting products were prone to racemization and epimerization respectively when dissolved in protic solvents such as water and MeOH, leading to a 1:1 mixture of enantiomers or epimers (FIG. 5).

In the presence of D₂O at room temperature, deuterium was incorporated at C-2 confirming a fast enolization followed by β-elimination and ring closure. Analog 12 was stable only in very acidic conditions (pH≤3 in H₂O), while epimerization was extremely fast in basic conditions. The incorporation of deuterium is complete in 30 min at pH=10, and in 3 hours at pH=7.

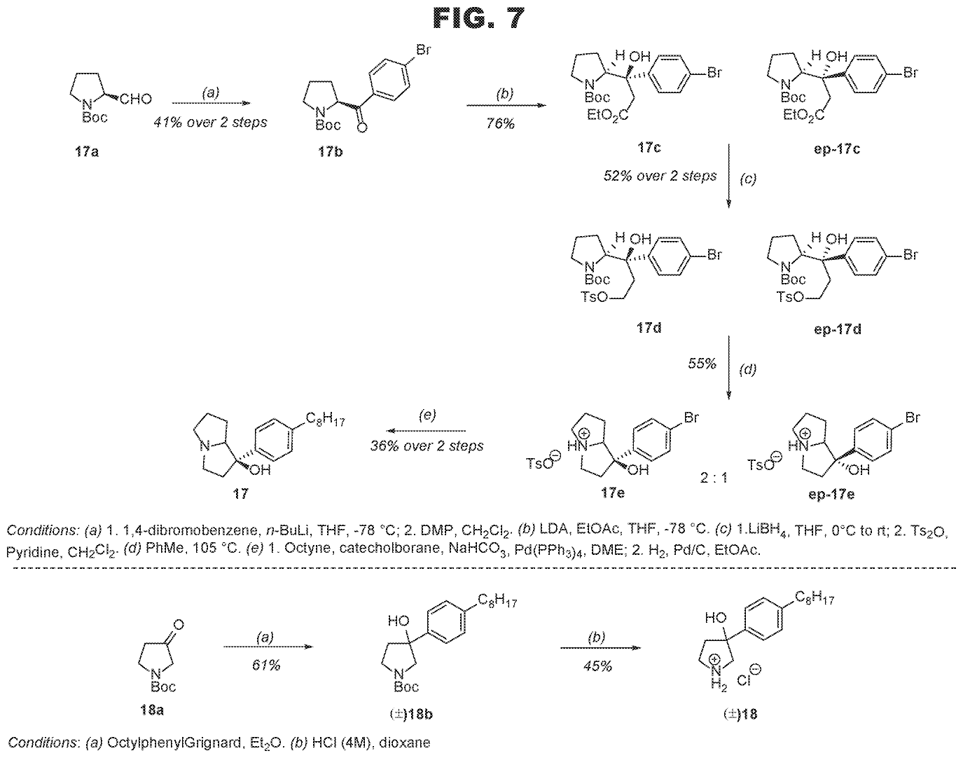

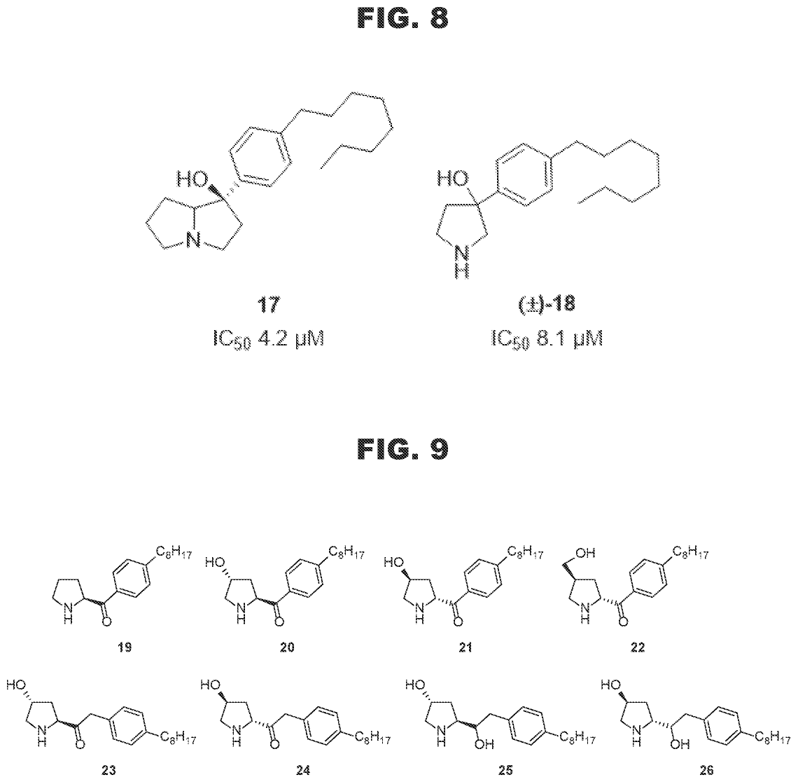

When the keto group was further removed from the pyrrolidine ring by extending the chain length as in 13, the desired ketone was found to be in equilibrium with an azabicyclic salt resulting from intramolecular iminium ion formation (2:1 mixture in H₂O) (13e), which upon reduction with NaBH₄ led to 14 (FIG. 6). The same behavior was observed in the case of compound 15. Surprisingly, the new bicyclic derivative 14 maintained a reasonable cytotoxic activity (IC₅₀ 12.4 μM). It was therefore decided to further investigate this new structural analog and we prepared the bicyclic enantiopure pyrrolizidine 17 bearing the aryloctyl appendage on C-3, in analogy with our lead compound 3 as well as the monocyclic variant 18, to check the effect of the substituent position and the nature of the azacycles (FIG. 7).

The synthesis was started from the readily available (S)-prolinal which was arylated and reoxidized to the corresponding ketone since the addition of the corresponding aryl Grignard on the Weinreb amide resulted in decomposition of the reagent without conversion of the substrate. Subsequent addition of ethyl acetate delivered 17c as a mixture of diastereomers which could not be separated at this stage (FIG. 7). The ester was further reduced to the alcohol and tosylated, which underwent a spontaneous deprotection/cyclisation process leading to the formation of the bicyclic structure 17e and its epimer epi-17e as tosylate salts in a ratio of 2:1.

The diastereomers were separated at this stage, delivering the major product in 17e in 38% yield. Suzuki coupling and hydrogenolysis of the alkene resulted in the formation of 17. The moderate yield over the two last steps was attributed to the highly sensitive benzylic alcohol decomposing easily under acidic conditions.

The racemic compound 18 was accessed from N-Boc 3-pyrrolidinone which was arylated with the octylphenyl Grignard and deprotected under acidic conditions.

### Repositioning the Keto Group in Extended C-2 Modified Analogs

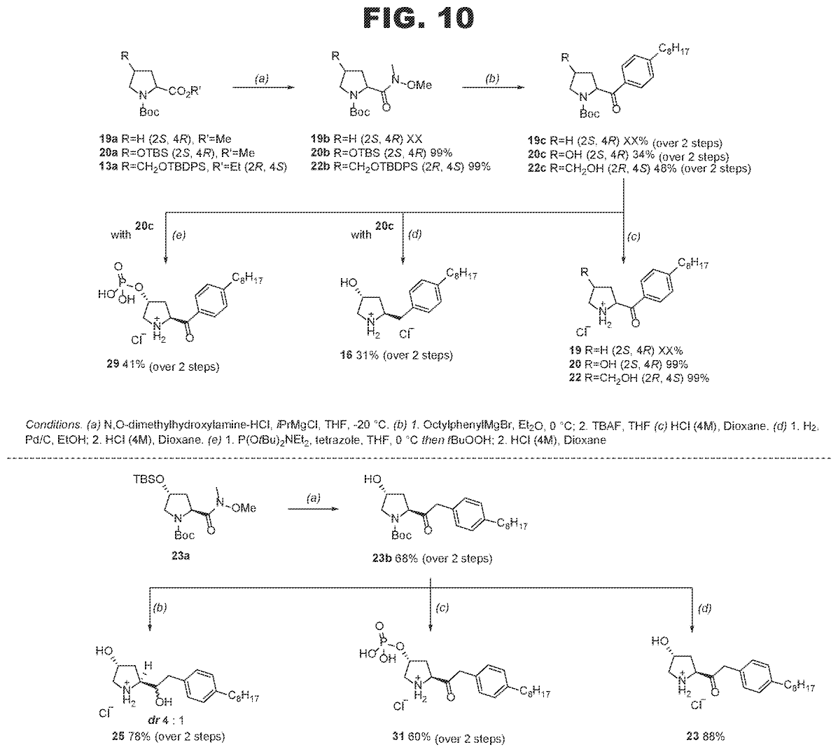

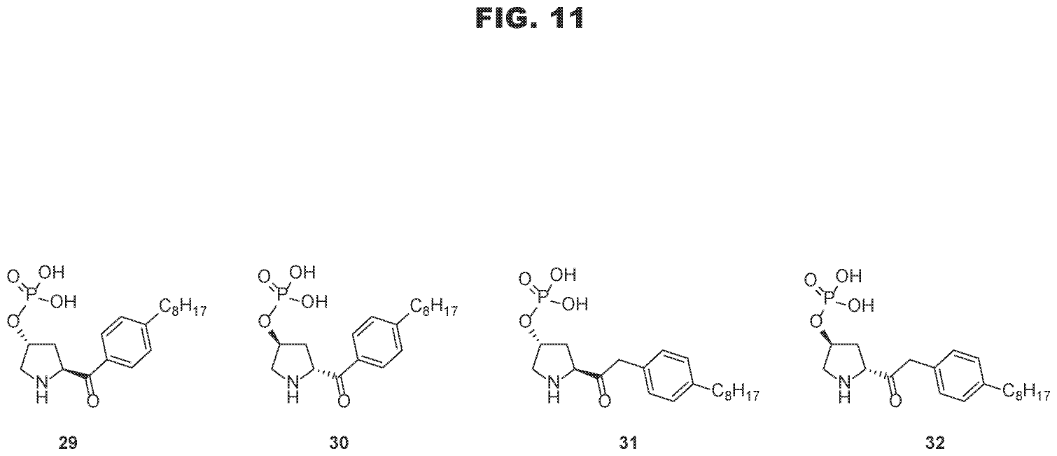

In considering an alternative position of the keto group in the chain, the keto group was placed on the alpha-position of C-2 branched aryloctyl pyrrolidine analogs (FIG. 9; Table 2). This would also avoid the partial epimerization issues encountered due to beta elimination and ring closure as described above, although the basicity of the pyrrolidine nitrogen might be expected to be diminished due the inductive effect of the carbonyl group.

TABLE 2
Cytotoxicity, nutrient transporter down-regulation and vacuolation profiles of β-aminoketone analogs

| | | | % CD98 down-regulation | | | Vacuolation score | | |
|---|---|---|---|---|---|---|---|---|
| Comp. | IC50 (μM) [95% Cl] | | 2.5 μM | 10 μM | 40 μM | 2.5 μM | 10 μM | 40 μM |
| 3 | 2.1 | [2.0, 2.2] | 42 | 66 | n.d. | ++ | +++ | n.d. |
| 19 | <40 | | −21 | −4 | 33 | 0 | 0 | +++ |
| 20 | 28.1 | [26.1, 30.2] | 2 | 9 | 48 | 0 | 0/+ | +++ |
| 21 | 15.3 | [14.6, 15.9] | 12 | 51 | 41 | 0 | +++ | +++ |
| 22 | 19.6 | [18.7, 20.5] | −0 | 19 | 61 | 0 | 0 | +++ |
| 23 | 18.6 | [15.5, 22.3] | 13 | 23 | 59 | 0 | 0 | +++ |
| 24 | 17.1 | [14.5, 20.2] | 11 | 16 | 54 | 0 | 0 | +++ |
| 25 | 3.0 | [2.6, 3.5] | 19 | 62 | n.d. | + | +++ | n.d. |
| 26 | 1.8 | [1.7, 2.0] | 9 | 62 | n.d. | 0 | ++ | n.d. |

In general, the cytotoxic activity of the C-2 ketoaryl derivatives was significantly reduced compared to 3 and to the corresponding β-keto analogs (compare Tables 1 and 2). This indicated that a C-2 carbonyl group adjacent to the pyrrolidine nitrogen atom was not well tolerated. However, the corresponding alcohols retained the activity of compound 3. Nevertheless, all of the C-2 keto analogs were able to down-regulate nutrient transporters at elevated concentrations. The same trend held for vacuolation, as 21 reached maximum vacuolation at 10 μM (Table 2). The negative influence of the α-keto group in the chain was further seen by the 5-10 fold decrease in cytotoxicity when comparing compounds 20 and 21 to 16. It is also notable that, at the concentration where it kills 50% of cells, 26 does not down-regulate nutrient transporter proteins or vacuolate suggesting an alternative mode of action.

Analogs 20, 22 and 23, as well as the corresponding reduction product 25, were synthetized as single enantiomers from the intermediates 20b, 22b and 23a (FIG. 10). The Weinreb amides 20b and 22b were prepared according to Toda et al. (N. Toda, et al., Org Lett. 5 (2003) 269-271, the disclosure of which is herein incorporated by reference) without epimerizing at the C-2 position. After the formation of the ketones by reaction with octylphenyl magnesium bromide, and removal of the O-protecting group using TBAF, intermediates 20c and 22c were converted individually into the α-ketoaryl pyrrolidines 20 and 22. Pd/C-catalyzed hydrogenation of the keto group in 22c led to N-Boc 16 in modest yield. Following a similar protocol, the extended ketone intermediate 23b was prepared, which was transformed to 23 and 25, the latter consisting of a 4:1 mixture of diastereomers (FIG. 10).

Phosphate esters 29 and 31 were prepared as well as their enantiomers (not shown) by standard methods (FIG. 10). These phosphates were tested to see if any exhibited cytotoxic activity. While it was not surprising that the phosphate esters 29, 30, 31, and 32 were totally inactive in downregulation and vacuolation tests compared to their hydroxy pyrrolidine ketone progenitors 20, 21, 23 and 24, as the charged phosphate should not be able to enter the cell, it was surprising to find that these analogs were cytotoxic at IC₅₀ 12.9 μM, 12.8 μM, 25.0 μM and 25.3 μM respectively (Table 3). The cytotoxicity of the phosphate esters over the unphosphorylated progenitors despite the absence of transporter loss or vacuolation suggests that they could act through a distinct mechanism, possibly targeting a receptor on the cell surface.

TABLE 3
Cytotoxicity, nutrient transporter down-regulation and vacuolation profiles of β-aminoketone phosphate analogs

| | | | % CD98 down-regulation | | | Vacuolation score | | |
|---|---|---|---|---|---|---|---|---|
| Comp. | IC50 (μM) [95% Cl] | | 2.5 μM | 10 μM | 40 μM | 2.5 μM | 10 μM | 40 μM |
| 3 | 2.1 | [2.0, 2.2] | 42 | 66 | n.d. | ++ | +++ | n.d. |
| 29 | 12.9 | [11.9, 14.0] | n.d. | 3 | −8 | 0 | 0 | 0 |
| 30 | 12.8 | [11.7, 14.0] | n.d. | −9 | −0 | 0 | 0 | 0 |
| 31 | 25.0 | [18.1, 34.4] | n.d. | 3 | 0 | n.d. | 0 | 0 |
| 32 | 25.3 | [19.5, 32.9] | n.d. | 5 | 8 | n.d. | 0 | 0 |

### Synthesis and Characterization of Compounds

All reactions involving moisture sensitive compounds were performed in flame-dried glassware under a positive pressure of dry, oxygen free, argon and in dry solvents. Anhydrous solvents were distilled under a positive pressure of argon before use and dried by standard methods. THF, ether, CH₂Cl₂ and toluene were dried by the SDS (Solvent Delivery System). Commercial grade reagents were used without further purification. Silica column chromatography was performed on 230-400 mesh silica gel. Thin layer chromatography (TLC) was carried out on glass-backed silica gel plates. Visualisation was effected by UV light (254 nm) or by staining with potassium permanganate solution, cerium ammonium molybdate or p-anisaldehyde followed by heating. ¹H and ¹³C NMR spectra were recorded on Bruker AV-400 and AV-500 MHz spectrometers at room temperature (298 K). Chemical shifts are reported in parts per million (ppm) referenced from CDCl₃(δ_{H}:7.26 ppm and δ_{C}:77.0 ppm). Coupling constants (J) are reported in Hertz (Hz). Multiplicities are given as multiplet (m), singlet (s), doublet (d), triplet (t), quartet (q), quintet (quin.) and broad (br.). Infrared spectra were recorded on a FT-IR spectrometer and are reported in reciprocal centimetres (cm⁻¹). Optical rotations were determined on an Anton Paar MCP 300 polarimeter at 589 nm. Specific rotations are given in units of 10⁻¹ deg cm² g⁻¹. High resolution mass spectra (HRMS) were performed by the “Centre regional de spectroscopie de masse de l'Université de Montreal” with electrospray ionisation (ESI) coupled to a quantitative time-of-flight (TOF) detector.

General procedure A for N-Boc deprotection: HCl (500 μL, 4M in dioxane, excess) was added to an N-Boc intermediate in dry dioxane. The reaction was stirred at rt until disappearance of the starting material by TLC analysis. The solution was then concentrated in vacuo in several cycles co-distilling with dry dioxane.

General procedure B for removal of silyl ethers: TBAF (1.1 eq., 1.0 M in THF) was added to a solution of silyl ether in dry THF (C=0.06 M). The reaction was then stirred at rt until disappearance of the starting material by TLC analysis. The solution was diluted with saturated aq. NaHCO₃ solution and EtOAc. The aqueous layer was extracted ×2 with EtOAc. The organic layers were combined, washed ×1 with brine, dried over Na₂SO₄, filtered, concentrated.

tert-Butyl (S)-2-(2-(4-octylphenyl)-2-oxoethyl)pyrrolidine-1-carboxylate (8b): Prepared according to general procedure C, starting from 8a (M. J. Bottomley, et al., J. Biol. Chem. 283 (2008) 26694-26704, the disclosure of which is herein incorporated by reference) (300 mg, 1.10 mmol). The crude was purified by flash column chromatography (EtOAc/hexane 1:6, Rf: 0.17) to give 8b as a colorless oil (274 mg, 62%). α²⁰_{D} −22.9 (c 1.3, CHCl₃). IR (neat), vₘₐₓ: 2924, 2854, 1680, 1606, 1455, 1391, 1365, 1277, 1169, 1116, 1012, 989, 772, 545 cm⁻¹. ¹H NMR (CDCl₃, 500 MHz, mixture of rotamers), 67 : 7.96-7.87 (m, 2H), 7.25 (d, J=7.5Hz, 2H), 4.34-4.29 (m, 1H), 3.74 (br. d, J=14.8 Hz, 0.5H), 3.47 (br. d, J=15.2 Hz, 0.5H), 3.40 (br. s, 1H), 3.32 (br. s, 1H), 2.85-2.73 (m, 1H), 2.64 (br. s, 2H), 2.03 (br. s, 1H), 1.90-1.79 (m, 2H), 1.75 (br. s, 1H), 1.64-1.57 (m, 2H), 1.45 (s, 9H), 1.30-1.22 (m, 10H), 0.86 (t, J=7.0 Hz, 3H) ppm. ¹³C NMR (CDCl₃, 125 MHz, mixture of rotamers), δ: 198.8, 198.3, 154.4, 154.3, 149.1, 148.7, 134.6, 128.7, 128.5, 128.4, 79.7, 79.2, 54.5, 54.3, 46.7, 46.2, 43.7, 43.0, 36.0, 31.9, 31.3, 31.1, 30.3, 29.4, 29.3, 29.2, 28.6, 23.6, 22.8, 22.6, 14.1 ppm. HRMS (ESI) calcd. for C₂₅H₄₀NO₃ (M+H)⁺ 402.30027, found 402.29965.

(S)-1-(4-Octylphenyl)-2-(pyrrolidin-2-yl)ethan-1-one hydrochloride (8): Prepared according to general procedure A, starting from 8b (100 mg, 0.25 mmol). The crude was purified by flash column chromatography (EtOH/CH₂Cl₂ 1:4, Rf: 0.45) to give product 8 as a white solid (84 mg, 99%). Note: the product racemized spontaneously when dissolved in MeOH or H₂O. For biological testing a portion of this solid was dissolved in the minimum amount of HPLC grade water, filtered (pore size=0.45 μm) and lyophilized. α²⁵D -39.1 (c 0.23, CHCl₃). IR (neat), vₘₐₓ: 2921, 2852, 1678, 1605, 1589, 1466, 1377, 1222, 1188, 1032, 976, 914, 822, 770, 569 cm⁻¹. ¹H NMR (CDCl₃, 400 MHz), δ: 9.52 (br. s, 2H), 7.86 (d, J=8.1 Hz, 2H), 7.18 (d, J=7.8 Hz, 2H), 4.17-4.09 (m, 1H), 3.89 (dd, J=18.4, 5.9 Hz, 1H), 3.50 (dd, J=18.4, 6.8 Hz, 1H), 3.40 (t, J=7.2 Hz, 2H), 2.61-2.57 (m, 2H), 2.37-2.29 (m, 1H), 2.10-1.95 (m, 2H), 1.81-1.70 (m, 1H), 1.59-1.54 (m, 2H), 1.30-1.24 (m, 10H), 0.88 (t, J=6.8 Hz, 3H) ppm. ¹³C NMR (CDCl₃, 125 MHz), δ: 196.8, 149.6, 133.6, 128.7, 128.4, 56.0, 45.0, 40.5, 36.0, 31.9, 31.0, 30.6, 29.7, 29.4, 29.3, 29.2, 23.6, 22.6, 14.1 ppm. HRMS (ESI) calcd. for C₂₀H₃₂NO (M)⁺ 302.24784, found 302.24782.

tert-Butyl (2S)-2-(2-hydroxy-2-(4-octylphenyl)ethyl)pyrrolidine-1-carboxylate (8c): NaBH₄ (4.9 mg, 0.13 mmol, 1.5 eq.) was added to a solution of 8b (35 mg, 0.087 mmol) in MeOH (3 mL) at 0° C. The resulting mixture was stirred for 2 hours at the same temperature. Afterwards, the reaction was quenched with brine (1 mL), the MeOH was removed in vacuo and the product was extracted with EtOAc (4×4 mL). The organic layers were dried over Na₂SO₄, filtered and concentrated. The residue was purified by flash column chromatography (EtOAc/hexane 1:6, then EtOAc/hexane 1:4 Rf: 0.38 and 0.19) to give 8c diast1 (7 mg, 20%) and 8c diast2 (28 mg, 80%) as colorless oils. 8c diastl: α²⁰_{D} −8.0 (c 0.2, MeOH). IR (neat), vₘₐₓ: 3406, 2923, 2851, 1723, 1671, 1397, 1245, 1168, 1104, cm⁻¹. ¹H NMR (CDCl₃, 300 MHz), δ: 7.28 (d, J=7.9 Hz, 2H), 7.13 (d, J=8.0 Hz, 2H), 5.33 (br. s, 1H), 4.64-4.57 (m, 1H), 4.33-4.25 (m, 1H), 3.37 (t, J=6.6 Hz, 2H), 2.60-2.54 (m, 2H), 2.03-1.93 (m, 2H), 1.91-1.87 (m, 2H), 1.72-1.67 (m, 2H), 1.62-1.54 (m, 2H), 1.49 (s, 9H), 1.25 (br. s, 10H), 0.87 (t, J=6.8 Hz, 3H) ppm. ¹³C NMR (CDCl3, 75 MHz), δ: 156.7, 141.6, 141.5, 128.2, 125.6, 80.0, 69.8, 54.0, 46.6, 46.3, 35.6, 31.9, 31.5, 31.2, 29.7, 29.5, 29.3, 28.5, 23.6, 22.7, 14.1 ppm. HRMS (ESI) calcd. for C₂₅H₄₁NO₃Na (M+Na)⁺ 426.29787, found 426.29919. 8c diast2: α²⁰_{D} −52.5 (c 0.8, MeOH). IR (neat), vₘₐₓ: 3413, 2924, 2854, 1668, 1393, 1365, 1247, 1168, 1103, 849, 772, 557 cm⁻¹. ¹H NMR (CDCl₃, 300 MHz), δ: 7.26 (d, J=7.8 Hz, 2H), 7.13 (d, J=7.7 Hz, 2H), 4.74 (br. s, 1H), 4.10 (br. s, 1H), 3.31 (br. s, 2H), 2.60-2.55 (m, 2H), 2.14 (br. s, 1H), 2.05-1.93 (m, 1H), 1.89-1.79 (m, 2H), 1.69 (br. s, 2H), 1.61-1.54 (m, 2H), 1.46 (s, 9H), 1.30-1.26 (m, 10H), 0.87 (t, J=6.8 Hz, 3H) ppm. ¹³C NMR (CDCl₃, 75 MHz), δ: 155.4, 142.3, 141.6, 128.3, 125.5, 79.7, 72.5, 55.7, 46.4, 46.3, 35.6, 32.4, 31.9, 31.5, 29.7, 29.5, 29.3, 29.2, 28.5, 23.8, 22.6, 14.1 ppm. HRMS (ESI) calcd. for C₂₅H₄₁NO₃Na (M+Na)⁺ 426.29787, found 426.29907.

tert-Butyl (R)-2-(4-octylphenethyl)pyrrolidine-1-carboxylate (8c1): 8c (12 mg, 0.0297 mmol) was dissolved in EtOH (3 mL) and Pd/C (10%, 7 mg) was added to the resulting solution. The air was removed from the flask under vacuum and replaced with hydrogen (balloon). The reaction was vigorously stirred overnight at room temperature. Afterwards, the mixture was filtered through a celite pad, washing with EtOH. The collected solution was concentrated in vacuo, affording 8c1 as a colorless oil (9 mg, 78%). α²⁰_{D} −36.0 (c 0.45, CHCl₃). IR (neat), vₘₐₓ: 2924, 2853, 1694, 1514, 1455, 1391, 1364, 1254, 1169, 1100, 771 cm⁻¹. ¹H NMR (CDCl₃, 400 MHz, mixture of rotamers), δ: 7.09 (s, 4H), 3.85 (br. s, 0.4H), 3.75 (br. s, 0.6H), 3.41 (br. s, 0.8H), 3.32 (br. s, 1.2H), 2.58-2.54 (m, 4H), 2.14 (br. s, 0.4H), 2.04-1.87 (m, 1.6H), 1.85-1.80 (m, 2H), 1.72 (br. s, 1H), 1.63-1.55 (m, 3H), 1.45 (s, 9H), 1.32-1.24 (m, 10H), 0.88 (t, J=6.8 Hz, 3H) ppm.

¹³C NMR (CDCl₃, 75 MHz, major rotamer), δ: 154.6, 140.3, 139.2, 128.3, 128.1, 79.0, 56.9, 46.1, 36.4, 35.5, 32.4, 31.9, 31.6, 30.6, 29.7, 29.5, 29.4, 29.2, 28.6, 23.2, 22.7, 14.1 ppm. HRMS (ESI) calcd. for C₂₅H₄₁NO₂K (M+K)⁺ 426.27744, found 426.27543.

(R)-2-(4-Octylphenethyl)pyrrolidine hydrochloride (9): Prepared according to general procedure A, starting from 8c1 (9 mg, 0.023 mmol). The crude was purified by flash column chromatography (EtOH/CH₂Cl₂ 1:8, Rf: 0.16) to give product 9 as a white solid (7 mg, 93%). For biological testing a portion of this solid was dissolved in the minimum amount of HPLC grade water, filtered (pore size=0.45 pm) and lyophilized. α²⁵_{D} −4.0 (c 0.35, CHCl₃). IR (neat), vₘₐₓ: 2921, 2852, 2751, 1591, 1514, 1455, 1418, 1042, 815, 722, 554 cm⁻¹. ¹H NMR (CDCl₃, 500 MHz), δ: 9.69 (br. s, 1H), 9.19 (br. s, 1H), 7.12 (d, J=8.0 Hz, 2H), 7.05 (d, J=8.0 Hz, 2H), 3.56-3.48 (m, 1H), 3.44-3.37 (m, 1H), 3.35-3.30 (m, 1H), 2.80-2.74 (m, 1H), 2.71-2.65 (m, 1H), 2.55-2.52 (m, 2H), 2.37-2.30 (m, 1H), 2.15-2.08 (m, 1H), 2.06-1.97 (m, 2H), 1.96-1.88 (m, 1H), 1.72-1.64 (m, 1H), 1.59-1.53 (m, 2H), 1.29-1.25 (m, 10H), 0.87 (t, J=7.0 Hz, 3H) ppm. ¹³C NMR (CDCl₃, 125 MHz), δ: 140.9, 137.2, 128.6, 128.3, 60.0, 44.8, 35.5, 34.0, 32.5, 31.9, 31.6, 30.4, 29.7, 29.5, 29.4, 29.3, 23.5, 22.7, 14.1 ppm. HRMS (ESI) calcd. for C₂₀H₃₄N (M)⁺ 288.26858, found 288.26992.

a tert-Butyl (2S)-2-(2-methoxy-2-(4-octylphenyl)ethyl)pyrrolidine-1-carboxylate (8c2): NaH (1.3 mg, 60% dispersion in mineral oil, 0.033 mmol, 1.2 eq.) was added to a solution of 8c diast2 (11 mg, 0.027 mmol) in dry THF (1 mL) at 0° C. The resulting mixture was stirred at the same temperature for 1 h, before adding methyl iodide (5 □L, 0.081 mmol, 3 eq.). Then, the reaction was stirred at room temperature for 3 h, before being quenched with water (1 mL). The product was extracted with EtOAc (3×2 mL) and the combined organic layers were dried over MgSO₄, filtered and concentrated. The residue was purified by flash column chromatography (EtOAc/hexane 1:6, Rf: 0.16) to give 8c2 as a colorless oil (7 mg, 64%). α²⁵_{D} −77.1 (c 0.35, CHCl₃). IR (neat), vₘₐₓ: 2924, 2854, 1692, 1454, 1391, 1364, 1251, 1170, 1102, 771 cm⁻¹. ¹H NMR (CDCl₃, 500 MHz, mixture of rotamers), δ: 7.23-7.13 (m, 4H), 4.11 (br. s, 1H), 4.02 (br. s, 0.5H), 3.93 (br. s, 0.5H), 3.39 (br. s, 0.5H), 3.29 (br. s, 1.5H), 3.16 (s, 3H), 2.60-2.57 (m, 2H), 2.27 (br. s, 1H), 1.82-1.73 (m, 2H), 1.62-1.56 (m, 3H), 1.46 (s, 9H), 1.30-1.26 (m, 10H), 0.88 (t, J=6.9 Hz, 3H) ppm. ¹³C NMR (CDCl₃, 125 MHz, mixture of rotamers), δ: 154.5, 142.2, 139.5, 128.4, 126.5, 81.6, 79.0, 78.7, 56.3, 54.7, 46.3, 46.1, 42.6, 35.7, 31.9, 31.5, 30.9, 30.3, 29.7, 29.5, 29.3, 29.2, 28.6, 23.7, 23.2, 22.6, 14.1 ppm. HRMS (ESI) calcd. for C₂₆H₄₄NO₃ (M+H)⁺ 418.33210, found 418.33141.

(2S)-2-(2-Methoxy-2-(4-octylphenyl)ethyl)pyrrolidine hydrochloride (10): Prepared according to general procedure A, starting from 8c2 (6 mg, 0.014 mmol). The crude was purified by flash column chromatography (EtOH/CH₂Cl₂ 1:10, Rf: 0.20) to give product 10 as a white solid (3 mg, 60%). For biological testing a portion of this solid was dissolved in the minimum amount of HPLC grade water, filtered (pore size=0.45 μm) and lyophilized. α²⁵_{D} −55.0 (c 0.20, CHCl₃). IR (neat), vₘₐₓ: 2921, 2851, 2766, 1459, 1107, 1033, 827, 722, 564 cm⁻¹. ¹H NMR (CDCl₃, 500 MHz), δ: 10.53 (br. s, 1H), 8.54 (br. s, 1H), 7.19 (d, J=8.1 Hz, 2H), 7.14 (d, J=8.1 Hz, 2H), 4.35 (dd, J=10.2, 2.9 Hz, 1H), 3.91 (br. s, 1H), 3.51-3.46 (m, 1H), 3.38-3.33 (m, 1H), 3.20 (s, 3H), 2.59-2.56 (m, 2H), 2.28-2.21 (m, 2H), 2.07-2.03 (m, 2H), 1.95-1.92 (m, 1H), 1.73-1.67 (m, 1H), 1.61-1.55 (m, 2H), 1.30-1.24 (m, 10H), 0.87 (t, J=7.0 Hz, 3H) ppm. ¹³C NMR (CDCl₃, 125 MHz), δ: 143.2, 137.3, 128.7, 126.4, 82.4, 58.9, 56.5, 44.5, 40.1, 35.7, 31.9, 31.4, 30.7, 29.7, 29.5, 29.3, 29.2, 23.1, 22.6, 14.1 ppm. HRMS (ESI) calcd. for C₂₁H₃₆NO (M)⁺ 318.27914, found 318.28009.

(2S,4R)-1-(tert-Butoxycarbonyl)-4-((tert-butyldimethylsilyl)oxy)pyrrolidine-2-carboxylic acid (11a2): 11a1 (200 mg, 0.56 mmol, 1.0 eq.) was dissolved in MeOH (1 mL) and an aqueous LiOH (330 μL, 1M, 1.5 eq.) was added. The solution was stirred at 45° C. for 3h whereby TLC analysis indicated that the reaction had gone to completion. A 5% (w/w) aqueous HCl solution was added dropwise until pH=2, whereby a white precipitate was formed. The mixture was extracted with Et₂O (2×5 mL). The resulting organic layer was collected, dried over Na₂SO₄, filtered and concentrated to afford 11a2 as an incolore oil which was brought to the next step without further purification (128 mg, 66%). α²⁰_{D} −67.31 (c 0.21, CHCl₃).

tert-Butyl(2S,4R)-4-((tert-butyldimethylsilyl)oxy)-2-(2-methoxy-2-oxoethyl)pyrrolidine-1-carboxylate (11a): 11a2 was synthesized in accordance with the procedure from 12a (120 mg, 0.35 mmol). The residue was purified by flash column chromatography (hexane/EtOAc 8:2 Rf: 0.28) to give 11a as a colorless oil (77 mg, 59% over 2 steps). α²⁰_{D} −74.10 (c 0.78, CHCl₃). IR (neat), vₘₐₓ: 2929, 1739, 1693, 1472, 1152, 1108, 853, 774 cm⁻¹. ¹H NMR (CDCl³, 500 MHz, mixture of rotamers), δ: 4.32-4.28 (p, J=4.3 Hz, 1H), 4.24-4.16 (m, 1H), 3.65 (s, 3H), 3.43-3.33 (m, 2H), 2.99-2.86 (dd, J=11.1, 4.6 Hz, 1H), 2.37 (m, 1H), 2.09 (m, 1H), 1.84 (m, 1H), 1.44 (s, 9H), 0.85 (s, 9H), 0.04 (s, 6H) ppm. ¹³C NMR (CDCl₃, 125 MHz, mixture of rotamers), δ: 172.0, 171.9, 155.0, 154.9, 79.9, 79.5, 70.2, 69.6, 55.1, 54.7, 53.0, 51.6, 41.2, 40.5, 39.6, 38.9, 28.6, 25.8, 18.1 ppm. HRMS (ESI) calcd. for C₂₅H₄₀NO₃ (M+H)⁺ 374.23680, found 374.23637.

tert-Butyl(2S,4R)-4-((tert-butyldimethylsilyl)oxy)-2-(2-(methoxy(methyl)amino)-2-oxoethyl)pyrrolidine-1-carboxylate (11b): 11b was synthesized in accordance with the procedure from 12b (30.0 mg, 0.08 mmol). The residue was purified by flash column chromatography (hexane/EtOAc 7:3 Rf: 0.35) to give 11b as a colorless oil (26 mg, 81%). α²⁰_{D} −56.60 (c 0.58, CHCl₃). IR (neat), vₘₐₓ: 2954, 1692, 1390, 1252, 1156, 835 cm⁻¹. ¹H NMR (CDCl₃, 500 MHz, mixture of rotamers), δ: 4.34-4.26 (m, 2H), 3.68 (s, 3H), 3.43-3.32 (m, 2H), 3.16-3.02 (m, 4H), 2.46 (bs, 1H), 2.12 (bs, 1H), 1.87 (bs, 1H), 1.45 (s, 9H), 0.85 (s, 9H), 0.04 (s, 6H) ppm. ¹³C NMR (CDCl₃, 125 MHz, mixture of rotamers), δ: 172.7, 172.4, 155.0, 79.7, 79.3, 70.3, 69.7, 61.4, 55.1, 54.5, 53.1, 41.4, 40.6, 37.7, 36.7, 32.1, 32.1, 28.6, 28.5, 25.9, 25.9, 18.1 ppm. HRMS (ESI) calcd. for C₂₅H₄₀NO₃ (M+H)⁺ 403.26230, found 403.26277.

tert-Butyl (2S,4R)-4-hydroxy-2-(2-(4-octylphenyl)-2-oxoethyl)pyrrolidine-1-carboxylate (11c1): 11c was synthesized in accordance with the general procedure C (11 mg, 0.02 mmol). 11c was obtained as a yellow oil which was submitted to general procedure B without further purification. The resulting residue was purified by flash column chromatography (hexane/EtOAc 8:2 Rf:0.33) to give 11c1 as a yellow oil (5 mg, 63% over 2 steps). α²⁰_{D} +50.40 (c 0.25, CHCl₃). IR (neat), vₘₐₓ: 2953, 1856, 1783, 1251, 932, 704 cm⁻¹. ¹H NMR (CDCl₃, 500 MHz, mixture of rotamers), δ: 7.89 (bs, 2H), 7.26-7.24 (m, 2H), 4.44-4.40 (m, 2H), 3.90-3.87 (m, 0.57 H), 3.67.3.60 (m, 1H), 3.46 (m, 0.64H), 2.89-2.84 (dd, J=15.5, 9.6 Hz, 1H), 2.65-2.62 (m, 2H), 2.22 (m, 2H), 1.90 (bs, 1H), 1.62-1.59 (m, 2H), 1.45 (s, 9H), 1.32-1.23 (m, 10H), 0.88-0.85 (t, 3H) ppm. ¹³C NMR (CDCl₃, 125 MHz, mixture of rotamers), δ: 198.9, 198.2, 171.3, 154.9, 149.3, 149.1, 134.6, 128.8, 128.5, 80.3, 79.7, 69.9, 69.4, 60.5, 54.9, 54.6, 53.3, 43.3, 40.3, 36.1, 32.0, 31.2, 29.5, 29.4, 29.3, 28.6, 22.8, 21.2, 14.3, 14.2 ppm. HRMS (ESI) calcd. for C₂₅H₄₀NO₃ (M+H)⁺ 418.29519, found 418.29710.

(2S,4R)-4-Hydroxy-2-(2-(4-octylphenyl)-2-oxoethyl)pyrrolidin-1-ium chloride (11): 11 was synthesized in accordance with the general procedure A (21 mg, 0.05 mmol). 11 was obtained as a white solid (13 mg, 75%). IR (neat), vₘₐₓ: 3310, 2921, 1669, 1605, 1277 cm⁻¹. ¹H NMR (CDCl₃, 500 MHz, mixture of diastereomers), δ: 7.71-7.70 (d, J=8.2 Hz, 2H), 7.00-6.99 (d, J=8.0 Hz, 2H), 4.51 (s, 1H), 4.07-4.04 (dd, J=11.3, 6.4 Hz, 1H major isomer), 3.99-3.96 (dd, J=9.0, 6.2 Hz, 1H minor isomer), 3.41-3.24 (m, 2H), 2.37-2.05 (m, 3H), 1.77-1.63 (m, 1H), 1.36 (m, 2H), 1.18-1.14 (m, 10H), 0.78-0.76 (t, 3H) ppm. ¹³C NMR (CDCl₃, 125 MHz, mixture of diastereomers), δ: 198.5, 149.1, 133.5, 128.5, 128.5, 69.1, 69.1, 54.3, 54.0, 52.8, 38.5, 37.7, 35.6, 31.8, 30.8, 29.4, 29.4, 29.3, 22.6, 13.8 ppm. HRMS (ESI) calcd. for C₂₅H₄₀NO₃ (M+H)⁺ 318.24276, found 318.24284.

1-(tert-butyl)2-ethyl(2R,4R)-4-(((tert-butyldiphenylsilyl)oxy)methyl)-5-oxopyrrolidine-1,2-dicarboxylate (12a3) and 1-(tert-butyl)2-ethyl(2R,4S)-4-(((tert-butyldiphenylsilyl)oxy)methyl)-5-oxopyrrolidine-1,2-dicarboxylate (epi-12a3): HCl (84 mL, 0.2 N in H₂O, 16.8 mmol, 1 eq.) was added dropwise to a solution of 12a1 (See N.C. Hait et al, Nat. Neuro. 17 (2014) 971-980, the disclosure of which is herein incorporated by reference (5.25 g, 16.8 mmol) in MeOH (125 mL) and the resulting solution was stirred at room temperature for 1 h. Afterwards, a small amount of bromocresol green was added to monitor the pH of the solution and NaBH₃CN (2.11 g, 33.6 mmol, 2 eq.) was added portion wise (4 portions over 2 h). Meanwhile, the pH of the solution had been corrected by adding a few drops of HCl (0.2 N in H₂O), anytime the pH indicator had turned blue. HCl has always been added in the minimal amount necessary to make the indicator turn back to yellow. The reaction was stirred at room temperature for 48 hours, continuing monitoring and correcting the pH when needed. Eventually, a few drops of NaHCO₃ satd. solution were added until the indicator turned blue and the mixture was concentrated in vacuo to remove the organic solvent. Brine (130 mL) was added to the mixture and the product was extracted in EtOAc (3×130 mL). The organic layers were dried over Na₂SO₄, filtered and concentrated. The resulting residue was purified by flash column chromatography (MeOH/CH₂Cl₂ 1:20, Rf:0.24) to give the intermediate alcohol 12a2 (3.38 g, 70%) as a 2:1 mixture of diasteroisomers. This intermediate was redissolved in dry DMF (60 mL) and imidazole (2.41 g, 35.4 mmol, 3 eq.) was added to the resulting solution. Then, this mixture was cooled down to 0° C. and TBDPSCI (4.60 mL, 17.7 mmol, 1.5 eq.) was added dropwise. The resulting solution was stirred at room temperature for 3 h, before adding EtOH (1 mL) and stirring for additional 30 min. Eventually, the mixture was poured into water (400 mL) and the product was extracted into Et₂O (3×300 mL). The organic layers were dried over Na₂SO₄, filtered and concentrated. The resulting residue was purified by flash column chromatography (Et₂O/hexane 1:2 Rf:0.13 and 0.06, then Et₂O/hexane 1:1) to give 12a3 (2.98 g, 48%) and epi-12a3 (1.12 g, 18%) as colorless oils. The stereochemistry was assigned by performing NOESY experiments (through space coupling observed between 2-H and 4-H in epi-12a3) and in analogy with similar published products. (See N. C. Hait, et al., Oncogenesis 4 (2015) e156, the disclosure of which is herein incorporated by reference) 12a3: α²⁵_{D} +28.3 (c 2.7, CHCl₃). IR (neat), vₘₐₓ: 2931, 1792, 1746, 1718, 1472, 1428, 1369, 1313, 1278, 1188, 1151, 1111, 1007, 966, 913, 848, 822, 734, 702, 613, 504 cm⁻¹. ¹H NMR (CDCl₃, 500 MHz), δ: 7.66-7.62 (m, 4H), 7.43-7.37 (m, 6H), 4.62 (dd, J=9.7, 3.2 Hz, 1H), 4.24 (q, J=7.1 Hz, 2H), 4.03 (dd, J=10.2, 4.8 Hz, 1H), 3.80 (dd, J=10.2, 3.4 Hz, 1H), 2.82-2.77 (m, 1H), 2.46-2.40 (m, 1H), 2.17-2.12 (m, 1H), 1.50 (s, 9H), 1.30 (t, J=7.1 Hz, 3H), 1.03 (s, 9H) ppm. ¹³C NMR (CDCl₃, 125 MHz), δ: 173.3, 171.6, 149.3, 135.7, 135.5, 133.2, 132.6, 129.8, 127.7, 83.4, 62.9, 61.6, 57.6, 44.5, 27.8, 26.8, 25.2, 19.2, 14.1 ppm. HRMS (ESI) calcd. for C₂₉H₃₉NO₆SiNa (M+Na)⁺ 548.24389, found 548.24492. epi-12a3: α²⁵_{D}+ 7.7 (c 3.6, CHCl₃). IR (neat), vₘₐₓ: 2931, 1791, 1746, 1718, 1473, 1428, 1369, 1318, 1151, 1109, 1032, 970, 909, 822, 781, 734, 702, 613, 504 cm⁻¹. ¹H NMR (CDCl3, 500 MHz), δ: 7.66-7.61 (m, 4H), 7.44-7.35 (m, 6H), 4.50 (dd, J=8.9, 7.5Hz, 1H), 4.22-4.13 (m, 2H), 3.93 (dd, J=10.3, 6.7 Hz, 1H), 3.87 (dd, J=10.3, 4.1 Hz, 1H), 2.79 (dddd, J=9.5, 8.7, 6.7, 4.1 Hz, 1H), 2.52-2.45 (m, 1H), 2.16 (ddd, J=13.2, 8.7, 7.5Hz, 1H), 1.49 (s, 9H), 1.25 (t, J=7.1 Hz, 3H), 1.04 (s, 9H) ppm. ¹³C NMR (CDCl3, 125 MHz), δ: 172.7, 171.3, 149.2, 135.6, 135.5, 133.2, 132.9, 129.7, 127.7, 83.5, 62.3, 61.5, 57.6, 45.4, 27.8, 26.7, 24.3, 19.2, 14.0 ppm. HRMS (ESI) calcd. for C₂₉H₃₉NO₆SiNa (M+Na)⁺ 548.24389, found 548.24498.

1-(tert-Butyl) 2-ethyl (2R,4S)-4-(((tert-butyldiphenylsilyl)oxy)methyl)pyrrolidine-1,2-dicarboxylate (13a): LiEt₃BH (4.68 mL, 1 M in THF, 4.68 mmol, 1.2 eq.) was added dropwise to a solution of 12a3 (2.05 g, 3.90 mmol) in anhydrous THF (70 mL) at −78° C. under an argon atmosphere and the resulting solution was stirred at the same temperature for 30 min. Afterwards, the reaction was quenched with NaHCO₃ satd. sol. (20 mL) and allowed to reach 0° C., then a few drops of H₂O₂ 30% were added and the mixture was stirred at 0° C. for 20 min. Eventually, the organic solvent was removed under vacuo and the remaining aqueous layer was extracted with CH₂Cl₂ (3×120 mL). The combined organic layers were dried over Na₂SO₄, filtered and concentrated, affording a colorless oil. This intermediate hemiaminal was redissolved in anhydrous CH₂Cl₂ (70 mL) and Et₃SiH (1.25 mL, 7.8 mmol, 2 eq.) was added to the solution under an argon atmosphere. The resulting mixture was cooled down to −78° C. and BF₃OEt₂(481 □L, 3.90 mmol, 1 eq.) was added dropwise. The reaction was stirred at −78° C. for 30 min, before adding NaHCO₃ satd. sol. (20 mL) and allowing the mixture to reach room temperature. The product was extracted with CH₂Cl₂ (3×120 mL) and the organic extracts were dried over Na₂SO₄, filtered and concentrated. The residue was purified by flash column chromatography (EtOAc/hexane 1:6, Rf: 0.24) to give 13a as a colorless oil (1.52 g, 76%). α²⁵_{D}⁺ 24.7 (c 1.5, CHCl₃). IR (neat), vₘₐₓ: 2931, 2858, 1744, 1699, 1473, 1427, 1389, 1365, 1257, 1188, 1110, 1030, 939, 870, 823, 741, 702, 611, 505 cm⁻¹. ¹H NMR (CDCl₃, 500 MHz, mixture of rotamers), δ: 7.65-7.63 (m, 4H), 7.44-7.37 (m, 6H), 4.34 (dd, J=7.9, 4.1 Hz, 0.4H), 4.24-4.14 (m, 2.6H), 3.73 (dd, J=10.6, 7.7 Hz, 0.6H), 3.64-3.59 (m, 2.4H), 3.29 (dd, J=10.6, 7.4 Hz, 0.6H), 3.23, (dd, J=10.5, 7.4 Hz, 0.4H), 2.60-2.51 (m, 1H), 2.13-2.04 (m, 1H), 2.03-1.98 (m, 1H), 1.47 (s, 3.6H), 1.42 (s, 5.4H), 1.30-1.25 (m, 3H), 1.06 (s, 3.6H), 1.05 (s, 5.4H) ppm. ¹³C NMR (CDCl₃, 125 MHz, mixture of rotamers), δ: 173.2, 172.9, 154.4, 153.7, 135.5, 133.4, 133.3, 129.7, 127.7, 79.8, 79.7, 64.8, 60.9, 60.8, 59.0, 58.7, 48.9, 48.8, 39.8, 38.9, 33.0, 32.2, 28.4, 28.3, 26.8, 19.2, 14.3, 14.1 ppm. HRMS (ESI) calcd. for C₂₉H₄₂NO₅Si (M+H)⁺ 512.28268, found 512.28059.

(2R,4S)-1-(tert-Butoxycarbonyl)-4-(((tert-butyldiphenylsilyl)oxy)methyl)pyrrolidine-2-carboxylic acid (12a4): NaOH (290 □L, 1 N in H₂O, 0.29 mmol, 1.5 eq.) was added to a solution of 13a (99 mg, 0.19 mmol) in MeOH (1.2 mL) and the resulting mixture was vigorously stirred for 24 h. Afterwards, the organic solvent was concentrated in vacuo and the residue was suspended in brine (20 mL). Afterwards, while gradually acidifying to pH=2 by adding HCl 0.2 N, the product was extracted with CH₂Cl₂ (6×20 mL). The organic layers were dried over Na₂SO₄, filtered and concentrated, affording 12a4 (93 mg, 99%) as a colorless solid. α²⁵_{D}⁺ 19.3 (c 0.9, MeOH). IR (neat), vₘₐₓ: 2929, 1699, 1390, 1366, 1162, 1108, 998, 906, 823, 739, 700, 608, 503 cm⁻¹. ¹H NMR (CD₃OD, 500 MHz, mixture of rotamers), δ: 7.74-7.66 (m, 4H), 7.47-7.37 (m, 6H), 4.30-4.21 (m, 1H), 3.66-3.57 (m, 3H), 3.37-3.33 (m, 1H), 2.57-2.54 (m, 1H), 2.17-2.11 (m, 1H), 2.06-2.03 (m, 1H), 1.48 (s, 3.6 H), 1.44 (s, 5.4 H), 1.06 (s, 9 H) ppm. ¹³C NMR (CD₃OD, 125 MHz, mixture of rotamers), δ: 175.9, 154.9, 154.6, 135.9, 135.3, 134.6, 133.1, 129.6, 129.5, 129.0, 127.5, 127.2, 80.0, 79.8, 64.8, 64.6, 59.5, 48.9, 48.6, 39.7, 38.9, 32.8, 32.1, 27.4, 27.2, 26.0, 25.8, 18.7, 18.5 ppm. HRMS (ESI) calcd. for C₂₇H₃₈NO₅SiNa (M+Na)⁺ 506.23332, found 506.23394.

tert-Butyl(2R,4S)-4-(((tert-butyldiphenylsilyl)oxy)methyl)-2-(2-methoxy-2-oxoethyl)pyrrolidine-1-carboxylate (12a): Et₃N (54 □L, 0.388 mmol, 2 eq.) and isobutylchloroformate (40 □L, 0.310 mmol, 1.6 eq.) were added to a solution of 12a4 (94 mg, 0.194 mmol) in anhydrous THF (2 mL) at 0° C. and the resulting mixture was stirred at room temperature for 1 h. Afterwards, the reaction was cooled down to 0° C. and a freshly prepared solution of CH₂N₂ in Et₂O was added dropwise until the resulting mixture remained bright yellow. Then, the reaction was stirred for 1 h at 0° C. and for 30 min at room temperature, adding further CH₂N₂ any time the mixture had turned back to colorless. Eventually, the flask was cooled down again to 0° C. and a 0.5 M solution of acetic acid in water was slowly added until the mixture turned colorless. Then, the layers were separated and the aqueous one was extracted with EtOAc (3×5 mL). The combined organic layers were washed with water (5 mL) and brine (5 mL), dried over Na₂SO₄, filtered and concentrated. The residue was purified by flash column chromatography (EtOAc/hexane 1:3, Rf: 0.21) to give the diazo intermediate as a pale yellow oil. This intermediate was redissolved in dry MeOH (2 mL) and a solution of silver benzoate (9 mg, 0.0388 mmol, 0.2 eq.) in Et₃N (54 □L, 0.388 mmol, 2 eq.) was added to this mixture under an argon atmosphere. Then, the flask was wrapped in aluminum foil and the reaction was refluxed for 2 h. Afterwards, the mixture was left to reach room temperature, filtered through a celite pad washing with abundant EtOAc and concentrated. The residue was purified by flash column chromatography (EtOAc/hexane 1:4, Rf: 0.27) to give 12a as a colorless oil (54 mg, 55% over two steps). α²⁵_{D}⁺ 21.2 (c 1.0, CHCl₃). IR (neat), vₘₐₓ: 2931, 2858, 1737, 1692, 1428, 1388, 1365, 1253, 1161, 1109, 823, 740, 702, 611, 504 cm⁻¹. ¹H NMR (CDCl₃, 500 MHz, mixture of rotamers), δ: 7.64-7.62 (m, 4H), 7.44-7.37 (m, 6H), 4.21 (br. s, 0.5H), 4.13 (br. s, 0.5H), 3.67 (s, 3H), 3.63-3.60 (m, 1H), 3.58-3.51 (m, 1.5H), 3.43 (br. s, 0.5H), 3.25-3.16 (m, 1H), 2.91, (d, J=14.2 Hz, 0.5H), 2.80 (d, J=14.6 Hz, 0.5H), 2.50-2.44 (m, 1H), 2.33 (dd, J=14.6, 9.7 Hz, 1H), 1.86 (br. s, 1H), 1.80-1.77 (m, 1H), 1.46 (s, 9H), 1.05 (s, 9H) ppm. ¹³C NMR (CDCl₃, 125 MHz, mixture of rotamers), δ: 171.9, 154.3, 154.1, 135.5, 133.5, 129.7, 127.9, 127.7, 79.6, 79.3, 65.3, 54.0, 51.6, 49.1, 39.3, 39.2, 38.7, 38.4, 33.7, 33.1, 28.5, 26.8, 19.2 ppm. HRMS (ESI) calcd. for C₂₉H₄₂NO₅Si (M+H)⁺ 512.28268, found 512.28235.

tert-Butyl(2R,4S)-4-(((tert-butyldiphenylsilyl)oxy)methyl)-2-(2-(methoxy(methyl)amino)-2-oxoethyl)pyrrolidine-1-carboxylate (12b): Isopropyl magnesium chloride (312 □L, 2 M in THF, 0.624 mmol, 6 eq.) was added dropwise to a solution of 12a (53 mg, 0.104 mmol) and N,O-dimethylhydroxylamine (30 mg, 0.312 mmol, 3 eq.) in dry THF (1 mL) at −20° C. The resulting mixture was allowed to reach 0° C. over 3 h, then, it was stirred at the same temperature overnight. Afterwards, the reaction was quenched by adding a few drops of water. Then, the mixture was filtered on a celite pad washing with abundant EtOAc and concentrated. The residue was purified by flash column chromatography (EtOAc/hexane 1:1, Rf:0.30) to give 12b as a colorless oil (47 mg, 84%). α²⁵_{D}⁺ 23.3 (c 0.6, CHCl₃). IR (neat), vₘₐₓ: 2931, 2857, 1690, 1385, 1364, 1256, 1162, 1109, 1000, 906, 870, 823, 739, 702, 611, 504 cm⁻¹. ¹H NMR (CDCl3, 500 MHz, mixture of rotamers), δ: 7.65-7.62 (m, 4H), 7.44-7.36 (m, 6H), 4.24 (br. s, 1H), 3.68 (s, 3H), 3.65-3.62 (m, 1H), 3.56 (br. s, 1.5H), 3.45 (br. s, 0.5H), 3.24 (br. s, 1H), 3.17 (s, 3H), 3.00, (d, J=14.7 Hz, 0.5H), 2.88 (d, J=13.6 Hz, 0.5H), 2.52-2.45 (m, 2H), 1.89-1.81 (m, 2H), 1.46 (s, 9H), 1.04 (s, 9H) ppm. ¹³C NMR (CDCl3, 125 MHz, mixture of rotamers), δ: 172.3, 154.2, 135.6, 135.5, 133.6, 129.7, 127.7, 79.5, 79.1, 65.4, 61.2, 54.0, 49.1, 39.2, 38.4, 36.8, 36.4, 33.8, 32.0, 28.5, 26.8, 19.2 ppm. HRMS (ESI) calcd. for C₃₀H₄₅N₂O₅Si (M+H)⁺ 541.30923, found 541.30687.

tert-Butyl (2R,4S)-4-(((tert-butyldiphenylsilyl)oxy)methyl)-2-(2-(4-octylphenyl)-2-oxoethyl)pyrrolidine-1-carboxylate (12c): Prepared according to general procedure C, starting from 12b (30 mg, 0.055 mmol). The crude was purified by flash column chromatography (EtOAc/hexane 1:10, Rf: 0.17) to give 12c as a colorless oil (25 mg, 68%). α²⁵_{D}⁺ 5.3 (c 0.3, CHCl₃). IR (neat), vₘₐₓ: 2924, 2853, 1671, 1606, 1515, 1458, 1390, 1366, 1175, 1111, 823, 739, 701, 611, 504 cm⁻¹. ¹H NMR (CDCl₃, 500 MHz, mixture of rotamers), δ: 7.95 (d, J=7.3 Hz, 1H), 7.90 (d, J=6.8 Hz, 1H), 7.64-7.61 (m, 4H), 7.43-7.35 (m, 6H), 7.27-7.26 (m, 2H), 4.38-4.34 (m, 1H), 3.72 (d, J=14.8 Hz, 0.5H), 3.61 (br. s, 1H), 3.58-3.53 (m, 1.5H), 3.50-3.43 (m, 1H), 3.29-3.25 (m, 0.5H), 3.22-3.19 (m, 0.5H), 2.88-2.78 (m, 1H), 2.65 (br. s, 2H), 2.54-2.48 (m, 1H), 1.87-1.75 (m, 2H), 1.65-1.59 (m, 2H), 1.47 (s, 4.5H), 1.45 (s, 4.5H), 1.31-1.26 (m, 10H), 1.03 (s, 9H), 0.88 (t, J=6.9 Hz, 3H) ppm. ¹³C NMR (CDCl₃, 125 MHz, mixture of rotamers), δ: 198.7, 198.3, 154.4, 154.2, 134.6, 133.5, 129.7, 128.7, 128.5, 128.4, 127.7, 79.7, 79.3, 65.4, 65.2, 54.4, 49.2, 43.9, 43.2, 39.2, 38.4, 36.0, 33.7, 32.7, 31.8, 31.1, 29.7, 29.4, 29.3, 29.2, 28.5, 26.8, 22.6, 19.2, 14.1 ppm. HRMS (ESI) calcd. for C₄₂H₆₀NO₄Si (M+H)⁺ 670.42861, found 670.42981.

tert-Butyl(2R,4S)-4-(hydroxymethyl)-2-(2-(4-octylphenyl)-2-oxoethyl)pyrrolidine-1-carboxylate (12c1): Prepared according to general procedure B, starting from 12c (14 mg, 0.021 mmol). The crude was purified by flash column chromatography (EtOAc/hexane 1:1, Rf:0.28) to give 12c1 as a colorless oil (9 mg, 99%). α²⁵_{D}⁺ 7.5 (c 0.4, CHCl₃). IR (neat), vₘₐₓ: 3439, 2924, 2854, 1673, 1606, 1394, 1366, 1255, 1173, 1123, 772, 558 cm⁻¹. ¹H NMR (CDCl₃, 500 MHz, mixture of rotamers), δ: 7.94 (d, J=7.7 Hz, 1H), 7.89 (d, J=7.9 Hz, 1H), 7.27-7.24 (m, 2H), 4.42-4.37 (m, 1H), 3.72 (d, J=15.5Hz, 0.5H), 3.63 (br. s, 1.5H), 3.58-3.49 (m, 1.5H), 3.47 (br. s, 0.5H), 3.26-3.23 (m, 0.5H), 3.16-3.13 (m, 0.5H), 2.91-2.80 (m, 1H), 2.65 (br. s, 2H), 2.55-2.46 (m, 1H), 1.89-1.80 (m, 2H), 1.64-1.59 (m, 2H), 1.46-1.40 (m, 9H), 1.31-1.25 (m, 10H), 0.88 (t, J=7.0 Hz, 3H) ppm. ¹³C NMR (CDCl₃, 125 MHz, mixture of rotamers), δ: 198.5, 198.0, 154.2, 148.8, 149.0, 134.5, 128.5, 128.4, 127.7, 79.7, 79.5, 65.2, 54.4, 49.2, 49.0, 43.9, 43.2, 39.2, 38.4, 36.0, 33.7, 32.7, 31.8, 31.1, 29.7, 29.4, 29.3, 29.2, 28.5, 26.8, 22.6, 14.1 ppm. HRMS (ESI) calcd. for C₂₆H₄₂NO₄ (M+H)⁺ 432.31084, found 432.30903.

2-((2R,4S)-4-(Hydroxymethyl)pyrrolidin-2-yl)-1-(4-octylphenyl)ethan-1-one hydrochloride (12): Prepared according to general procedure A, starting from 12c1 (8 mg, 0.019 mmol). The crude was purified by flash column chromatography (EtOH/CH₂Cl₂ 1:4, Rf:0.15) to give product 12 as a white solid (6 mg, 88%). Note: the product epimerized spontaneously on C-2 when dissolved in MeOH or H₂O, giving a 1:1 mixture of diasteroisomers. For biological testing a portion of the product was dissolved in the minimum amount of HPLC grade water, filtered (pore size=0.45 pm) and lyophilized. IR (neat), vₘₐₓ: 3376, 2922, 2852, 1675, 1605, 1570, 1465, 1378, 1282, 1184, 1039, 906, 815, 722, 549 cm⁻¹. ¹H NMR (CD₃OD, 500 MHz, 1:1 mixture of diasteroisomers), δ: 7.98 (d, J=8.3 Hz, 4H), 7.38 (d, J=8.3 Hz, 4H), 4.18-4.12 (m, 1H), 4.09-4.04 (m, 1H), 3.76-3.65 (m, 4H, partially deuterated), 3.63-3.58 (m, 2H), 3.47-3.39 (m, 4H, partially deuterated), 3.20-3.13 (m, 2H), 2.74-2.71 (m, 4H), 2.67-2.58 (m, 2H), 2.44-2.38 (m, 1H), 2.22-2.17 (m, 1H), 1.99 (dt, J=13.5, 8.4 Hz, 1H), 1.70-1.62 (m, 5 H), 1.36-1.30 (m, 20H), 0.91 (t, J=7.0 Hz, 6H) ppm. ¹³C NMR (CD₃OD, 125 MHz, 1:1 mixture of diasteroisomers), δ: 197.1, 149.7, 133.6, 128.6, 128.0, 62.4, 61.9, 56.2, 55.3, 39.6, 39.0, 35.5, 32.9, 32.6, 31.6, 30.9, 29.1, 29.0, 28.9, 22.3, 13.0 ppm. HRMS (ESI) calcd. for C₂₁ H₃₄NO₂ (M)⁺ 332.25841, found 332.25898.

tert-Butyl(2R,4S)-4-(((tert-butyldiphenylsilyl)oxy)methyl)-2-((E)-3-methoxy-3-oxoprop-1-en-1-yl)pyrrolidine-1-carboxylate (13a1): DIBAL-H (760 □L, 1 M in CH₂Cl₂, 0.76 mmol, 2 eq.) was added dropwise to a solution of 13a (194 mg, 0.38 mmol), in dry CH₂Cl₂ (3.5 mL) at −78° C. The resulting mixture was stirred at the same temperature for 2 hours, before quenching the reaction with MeOH (100 □L). Then, the solution was allowed to reach room temperature and a 2 M solution of potassium sodium tartrate in water (3.5 mL) was added. The resulting mixture was vigorously stirred at room temperature for 30 min, before separating the layers. The aqueous one was extracted with CH₂Cl₂ (3×7 mL) and the combined organic layers were dried over MgSO₄, filtered and concentrated, affording a colorless oil. This intermediate aldehyde was redissolved in dry CH₂Cl₂ (3.5 mL) and methyl(triphenylphosphoranylidene)acetate (191 mg, 0.57 mmol, 1.5 eq.) was added at 0° C. The resulting solution was stirred for 1 h at 0° C. and for 1 h at room temperature, before being cooled down again to 0° C. and quenched with NH₄Cl satd. sol. (3.5 mL). The layers were separated and the aqueous one was extracted with CH₂Cl₂ (3×7 mL). The combined organic layers were washed with water (7 mL) and brine (7 mL), dried over Na₂SO₄, filtered and concentrated. The residue was purified by flash column chromatography (EtOAc/hexane 1:4, Rf: 0.30) to give 13a1 as a colorless oil (143 mg, 72%). α²⁵_{D}⁺36.4 (c 1.1, CHCl₃). IR (neat), vₘₐₓ: 2931, 2858, 1725, 1693, 1428, 1387, 1364, 1265, 1162, 1107, 978, 861, 823, 740, 701, 611, 503 cm⁻¹. ¹H NMR (CDCl₃, 500 MHz, mixture of rotamers), δ: 7.65-7.62 (m, 4H), 7.43-7.37 (m, 6H), 6.87-6.97 (m, 1H), 5.83 (t, J=14.5Hz, 1H), 4.53 (br. s, 0.4H), 4.37 (br. s, 0.6H), 3.75 (s, 1.8H), 3.73 (s, 1.2H), 3.61 (d, J=6.3 Hz, 2H), 3.60-3.57 (m, 0.6H), 3.50-3.46 (m, 0.4H), 3.27 (t, J=9.4 Hz, 0.6H), 3.21-3.18 (m, 0.4H), 2.50-2.41 (m, 1H), 1.97-1.86 (m, 1H), 1.80 (dd, J=6.4, 2.5Hz, 0.6H), 1.78 (dd, J=6.4, 2.5Hz, 0.4H), 1.46 (s, 5.4H), 1.41 (s, 3.6H), 1.05 (s, 9H) ppm. ¹³C NMR (CDCl₃, 125 MHz, mixture of rotamers), δ: 166.8, 154.2, 154.1, 148.7, 148.4, 135.5, 133.3, 129.7, 127.9, 127.7, 120.0, 79.7, 65.0, 64.9, 57.7, 57.4, 51.6, 49.0, 48.9, 39.3, 38.4, 34.1, 33.4, 28.4, 28.2, 26.8, 19.2 ppm. HRMS (ESI) calcd. for C₃₀H₄₂NO₅Si (M+H)⁺ 524.28270, found 524.28136.

tert-Butyl(2S,4S)-4-(((tert-butyldiphenylsilyl)oxy)methyl)-2-(3-methoxy-3-oxopropyl)pyrrolidine-1-carboxylate (13b): 13a1 (122 mg, 0.23 mmol) was dissolved in MeOH (9 mL) and Pd/C (10%, 29 mg) was added to the resulting solution. The air was removed from the flask under vacuum and replaced with hydrogen (balloon). The reaction was vigorously stirred overnight at room temperature. Afterwards, the mixture was filtered through a celite pad, washing with MeOH. The collected solution was concentrated in vacuo, affording 13b as a colorless oil (122 mg, 99%). α²⁰_{D}⁺ 20.9 (c 0.9, CHCl3). IR (neat), vₘₐₓ: 2931, 2857, 1737, 1690, 1427, 1388, 1364, 1254, 1169, 1109, 823, 739, 701, 610, 504 cm⁻¹. ¹H NMR (CDCl₃, 500 MHz, mixture of rotamers), δ: 7.70-7.63 (m, 4H), 7.44-7.37 (m, 6H), 3.88 (br. s, 0.5H), 3.80 (br. s, 0.5H), 3.67 (s, 3H), 3.58 (d, J=6.2 Hz, 2H), 3.49-3.43 (m, 0.5H), 3.40-3.35 (m, 0.5H), 3.29-3.24 (m, 0.5H), 3.22-3.17 (m, 0.5H), 2.54-2.48 (m, 1H), 2.32 (br. s, 2H), 2.04-1.92 (m, 1H), 1.75-1.66 (m, 3H), 1.46 (s, 9H), 1.05 (s, 9H) ppm. ¹³C NMR (CDCl₃, 125 MHz, mixture of rotamers), δ: 173.9, 173.7, 154.7, 154.5, 135.5, 133.5, 129.7, 127.7, 79.4, 79.1, 65.6, 65.4, 56.5, 51.5, 48.9, 39.5, 38.7, 33.4, 32.8, 31.1, 30.2, 30.0, 28.5, 26.8, 19.2 ppm. HRMS (ESI) calcd. for C₃₀H₄₄NO₅Si (M+H)⁺ 526.2983, found 526.2993.

tert-Butyl(2S,4S)-4-(((tert-butyldiphenylsilyl)oxy)methyl)-2-(3-(methoxy(methyl)amino)-3-oxopropyl)pyrrolidine-1-carboxylate (13b1): Prepared as reported for 12b, starting from 13b (122 mg, 0.23 mmol). The crude was purified by flash column chromatography (EtOAc/hexane 1:1, Rf:0.28) to give 13b1 as a colorless oil (111 mg, 87%). α²⁵_{D}⁺ 20.2 (c 1.1, CHCl₃). IR (neat), vₘₐₓ: 2930, 2857, 1688, 1386, 1364, 1254, 1175, 1109, 997, 823, 740, 702, 611, 504 cm⁻¹. ¹H NMR (CDCl₃, 500 MHz, mixture of rotamers), δ: 7.65-7.63 (m, 4H), 7.44-7.36 (m, 6H), 3.92 (br. s, 0.5H), 3.83 (br. s, 0.5H), 3.68 (s, 3H), 3.59 (d, J=6.1 Hz, 2H), 3.51-3.45 (m, 0.5H), 3.43-3.37 (m, 0.5H), 3.30-3.25 (m, 0.5H), 3.17 (br. s, 3.5H), 2.57-2.51 (m, 1H), 2.43 (br. s, 2H), 1.94 (br. s, 1H), 1.71 (br. s, 3H), 1.45 (s, 9H), 1.04 (s, 9H) ppm. ¹³C NMR (CDCl₃, 125 MHz, mixture of rotamers), δ: 174.4, 174.2, 154.7, 135.5, 133.5, 129.6, 127.7, 79.2, 78.9, 65.7, 65.5, 61.1, 56.8, 48.8, 39.5, 38.7, 33.6, 33.0, 32.2, 30.0, 29.8, 29.7, 29.2 28.5, 26.8, 19.2 ppm.

HRMS (ESI) calcd. for C₃₁H₄₇N₂O₅Si (M+H)⁺ 555.3249, found 555.3268.

tert-Butyl(2S,4S)-4-(hydroxymethyl)-2-(3-(4-octylphenyl)-3-oxopropyl)pyrrolidine-1-carboxylate (13c1): Prepared by applying in sequence general procedures C and B, starting from 13b1 (34 mg, 0.061 mmol). The crude was purified by flash column chromatography (EtOAc/hexane 1:1, Rf: 0.20) to give 13c1 as a colorless oil (12 mg, 44% over two steps). α_{D}⁺ 6.3 (c 0.6, CHCl₃). IR (neat), vₘₐₓ: 3437, 2923, 2854, 1678, 1605, 1391, 1364, 1253, 1174, 1122, 770, 567 cm⁻¹. ¹H NMR (CDCl₃, 500 MHz, mixture of rotamers), δ: 7.87 (d, J=8.3Hz, 2H), 7.26-7.24 (m, 2H), 4.01 (br. s, 0.5H), 3.95 (br. s, 0.5H), 3.64-3.60 (m, 2H), 3.47-3.44 (m, 1H), 3.31-3.26 (m, 0.5H), 3.18-3.12 (m, 0.5H), 3.09-3.02 (m, 0.5H), 2.96 (br. s, 1.5H), 2.64 (t, J=7.6 Hz, 2H), 2.54 (br. s, 1H), 2.04 (br. s, 1H), 1.88-1.74 (m, 3H), 1.64-1.54 (m, 2H), 1.41 (s, 9H), 1.30-1.25 (m, 10H), 0.87 (t, J=7.0 Hz, 3H) ppm. ¹³C NMR (CDCl₃, 125 MHz, mixture of rotamers), δ: 199.8, 199.2, 154.8, 148.8, 148.6, 134.6, 128.6, 128.2, 79.5, 79.2, 64.8, 56.8, 49.0, 48.4, 39.6, 38.8, 36.0, 35.7, 35.3, 33.8, 33.2, 31.8, 31.1, 29.7, 29.6, 29.5, 29.4, 29.2, 29.1, 28.5, 27.5, 22.6, 14.1 ppm. HRMS (ESI) calcd. for C₂₇H₄₄NO₄ (M+H)⁺ 446.32650, found 446.32667.

3-(2S,4S)-4-(Hydroxymethyl)pyrrolidin-2-yl)-1-(4-octylphenyl)propan-1-one hydrochloride (13) and (2S)-2-(hydroxymethyl)-5-(4-octylphenyl)-1,2,3,6,7,7a-hexahydropyrrolizin-4-ium chloride (13e) : Prepared according to general procedure A, starting from 13c1 (6 mg, 0.013 mmol). The crude was triturated and washed with Et₂O, affording 13 as a white solid (4 mg, 80%). Note: in CD₃OD the product was slowly but completely converted into the bicyclic salt 13e. On the other hand, in D₂O the two species resulted in equilibrium, giving a mixture with a 2:1 constant ratio in favor of the open compound 13. For biological testing a portion of the product was dissolved in the minimum amount of HPLC grade water, filtered (pore size=0.45 μm) and lyophilized. 13: ¹H NMR (CD₃OD, 500 MHz), δ: 7.94 (d, J=8.3 Hz, 2H), 7.33 (d, J=8.3 Hz, 2H), 3.75-3.67 (m, 1H), 3.63-3.53 (m, 2H), 3.50 (dd, J=11.8, 8.2 Hz, 1H), 3.23 (dt, J=13.9, 6.9 Hz, 2H, partially deuterated), 3.12 (dd, J=11.8, 6.8 Hz, 1H), 2.71-2.67 (m, 2H), 2.66-2.60 (m, 1H), 2.21-2.01 (m, 3H), 1.91 (dt, J=13.5, 9.1 Hz, 1H), 1.68-1.61 (m, 2H), 1.33-1.29 (m, 10H), 0.89 (t, J=6.9 Hz, 3H) ppm. 13e: IR (neat), vₘₐₓ: 3410, 2923, 2853, 1645, 1605, 1456, 1417, 1373, 1296, 1190, 1045, 811, 566 cm⁻¹. ¹H NMR (CD₃OD, 500 MHz), δ: 7.93 (d, J=8.4 Hz, 2H), 7.56 (d, J=8.4 Hz, 2H), 5.09-5.00 (m, 1H), 4.23-4.17 (m, 1H), 4.14-4.06 (m, 2H, partially deuterated), 3.71 (dd, J=6.0, 1.3 Hz, 2H), 3.68-3.61 (m, 1H, partially deuterated), 3.02-2.95 (m, 1H), 2.80-2.76 (m, 2H), 2.60 (dt, J=12.7, 7.6 Hz, 1H), 2.28 (ddd, J=12.8, 7.4, 2.0 Hz, 1H), 2.13-2.02 (m, 1H), 1.98-1.90 (m, 1H), 1.72-1.65 (m, 2H), 1.35-1.29 (m, 10H), 0.89 (t, J=6.9Hz, 3H) ppm. ¹³C NMR (CD₃OD, 125 MHz), δ: 178.8, 152.6, 131.2, 129.5, 123.6, 75.7, 63.2, 51.4, 43.4, 41.1, 35.6, 31.6, 31.1, 30.7, 29.1, 29.0, 28.9, 26.6, 22.3, 13.0 ppm.

((2S,5S)-5-(4-Octylphenyl)hexahydro-1H-pyrrolizin-2-yl)methanol hydrochloride (14): NaBH₄ (0.5 mg, 0.012 mmol, 1.5 eq.) was added to a solution of 13e (3 mg, 0.008 mmol) in MeOH (300 □L) at 0° C. The resulting mixture was stirred for 1 hour at the same temperature. Afterwards, the reaction was quenched with HCl 1 N (20 □L) and concentrated. The crude was purified by flash column chromatography (MeOH/CH₂Cl₂ 1:4, Rf:0.58) to give product 14 as a white solid (3 mg, 99%, dr. 10:1). The stereochemistry was assigned by performing NOESY experiments (through space coupling observed between 2-H and 5-H in the major diastereoisomer). For biological testing the product was dissolved in the minimum amount of HPLC grade water, filtered (pore size=0.45 pm) and lyophilized. α²⁵_{D} −58.7 (c 0.15, MeOH). IR (neat), vₘₐₓ: 3417, 2923, 2853, 1518, 1456, 1089, 1037, 833, 535 cm⁻¹. ¹H NMR (CD₃OD, 500 MHz), δ: 7.49 (d, J=8.1 Hz, 2H), 7.32 (d, J=8.1 Hz, 2H), 4.52-4.46 (m, 1H), 4.42 (dd, J=10.9, 6.7 Hz, 1H), 3.69 (dd, J=11.1, 5.2 Hz, 1H), 3.62 (dd, J=11.1, 5.9 Hz, 1H), 3.45 (dd, J=11.8, 6.6 Hz, 1H), 3.09 (dd, J=11.8, 10.5Hz, 1H), 2.92-2.83 (m, 1H), 2.67-2.64 (m, 2H), 2.54-2.51 (m, 1H), 2.41-2.35 (m, 2H), 2.09-2.05 (m, 2H), 2.00-1.92 (m, 1H), 1.65-1.59 (m, 2H), 1.32-1.28 (m, 10H), 0.89 (t, J=6.9 Hz, 3H) ppm. ¹³C NMR (CD₃OD, 125 MHz), δ: 145.1, 130.2, 129.2, 127.9, 72.7, 68.6, 60.7, 54.1, 39.5, 35.2, 32.8, 32.7, 31.6, 31.2, 31.1, 29.1, 29.0, 28.9, 22.3, 13.0 ppm. HRMS (ESI) calcd. for C₂₂H₃₆NO (M)⁺ 330.2797, found 330.2793.

tert-Butyl (2S,4R)-4-((tert-butyldimethylsilyl)oxy)-2-((E)-3-methoxy-3-oxoprop-1-en-1-yl)pyrrolidine-1-carboxylate (15a1): 15a was synthesized in accordance with the procedure from 13a1 (200 mg, 0.56 mmol). The residue was purified by flash column chromatography (hexane/EtOAc 8:2 Rf:0.38) to give 15a1 as a colorless oil (150 mg, 69% over 2 steps). α²⁰_{D} −3.03 (c 3.15, CHCl₃). IR (neat), vₘₐₓ: 2977, 2926, 2855, 1701, 1396, 1260, 987, 753 cm⁻¹. ¹H NMR (CDCl3, 500 MHz, mixture of rotamers), δ: 6.85-6.84 (m, 1H), 5.90-5.75 (d, J=15.0 Hz, 1H), 4.57-4.47 (m, 1H), 4.35-4.33 (t, J=7.8 Hz, 1 H), 3.75 (s, 3H), 3.47-3.36 (m, 2H), 2.10-2.06 (m, 1H), 1.85-1.80 (m, 1H), 1.45 (s, 9H), 0.89 (s, 9H), 0.07 (s, 6H) ppm. ¹³C NMR (CDCl₃, 125 MHz, mixture of rotamers), δ: 200.3, 200.0, 166.9, 154.7, 149.3, 148.9, 128.5, 127.6, 127.0, 120.0, 79.9, 70.0, 69.7, 69.5, 56.9, 55.3, 54.8, 51.6, 41.5, 40.6, 36.9, 28.4, 28.2, 25.7, 17.9 ppm. HRMS (ESI) calcd. for C₂₅H₄₀NO₃ (M+H)⁺ 618.35301, found 618.35381.

tert-Butyl(2R,4R)-4-((tert-butyldimethylsilyl)oxy)-2-(3-methoxy-3-oxopropyl)pyrrolidine-1-carboxylate (15a): 15a was synthesized in accordance with the procedure from 13b (350 mg, 0.91 mmol). The residue was purified by flash column chromatography (hexane/EtOAc 8:2 Rf:0.33) to give 15a as a colorless oil (352 mg, 99%). α²⁰_{D} −29.09 (c 0.44, CHCl₃). IR (neat), vₘₐₓ: 2929; 1739; 1693; 1390; 1154; 833; 773 cm⁻¹. ¹H NMR (CDCl3, 500 MHz, mixture of rotamers), δ: 4.33-4.32 (q, 1H), 4.10-3.95 (m, 1H), 3.66 (s, 3H), 3.48-3.32 (m, 2H), 2.30 (bs, 2H), 2.07-1.96 (m, 2H), 1.78-1.71 (m, 2H), 1.45 (s, 9H), 0.86 (s, 9H), 0.05 (s, 6H) ppm. ¹³C NMR (CDCl₃, 125 MHz, mixture of rotamers), δ: 174.1, 155.3, 79.6,79.4, 70.5, 70.0, 55.6, 55.0, 54.7, 51.7, 40.8, 40.1, 31.0, 30.7, 30.5, 30.2, 28.6, 25.9, 18.1 ppm. HRMS (ESI) calcd. for C₂₅H₄₀NO₃ (M+H)⁺ 388.25140, found 388.25200.

tert-Butyl(2R,4R)-4-((tert-butyldimethylsilyl)oxy)-2-(3-(methoxy(methyl)amino)-3-oxopropyl)pyrrolidine-1-carboxylate (15a1): 15a1 was synthesized in accordance with the procedure from 12b (334.0 mg, 0.86 mmol). The residue was purified by flash column chromatography (hexane/EtOAc 6:4 Rf: 0.25) to give 15a1 as a colorless oil (328 mg, 92%). α²⁰_{D} −25.71 (c 0.35, CHCl₃). IR (neat), vₘₐₓ: 2928, 2854, 1738, 1390, 1156, 1110, 534, 774 cm⁻¹. ¹H NMR (CDCl3, 500 MHz, mixture of rotamers), δ: 4.36-4.34 (m, 1H), 3.97-3.95 (m, 1H), 3.68 (s, 3H), 3.39-3.33 (m, 2H), 3.17 (s, 3H), 2.41 (m, 2H), 1.99-1.97 (m, 2H), 1.77-1.73 (m, 2H), 1.45 (s, 9H), 0.86 (s, 9H), 0.05 (s, 6H), ppm. ¹³C NMR (CDCl₃, 125 MHz, mixture of rotamers), δ: 174.5, 155.4, 79.4, 70.3, 61.3, 55.9, 54.7, 40.6, 32.4, 30.3, 28.9, 28.6, 25.9, 18.1 ppm. HRMS (ESI) calcd. for C₂₅H₄₀NO₃ (M+H)⁺ 403.26230, found 403.26193.

tert-Butyl(2R,4R)-4-hydroxy-2-(3-(4-octylphenyl)-3-oxopropyl)pyrrolidine-1-carboxylate (15b1): 15b was synthesized in accordance with the general procedure C (100 mg, 0.24 mmol). 15b was obtained as a yellow oil which was submitted to general procedure B without further purification. The resulting residue was purified by flash column chromatography (hexane/EtOAc 4:6 Rf: 0.33) to give 15b1 as a yellow oil (61 mg, 59% over 2 steps). α²⁰_{D} −19.00 (c 0.40, CHCl3). IR (neat), vₘₐₓ: 2922, 1672, 1411 cm⁻¹.

¹H NMR (CDCl3, 500 MHz, mixture of rotamers), δ: 7.90-7.88 (d, J=8.2 Hz, 2H), 7.29-7.27 (d, J=9.3 Hz, 2H), 4.48-4.47 (m, 1H), 4.12-4.09 (m, 1H), 3.60-3.58 (d, J=11.7 Hz, 1H), 3.47-3.43 (dd, J=12.0, 4.6 Hz, 1H), 2.99-2.95 (m, 2H), 2.69-2.66 (t, J=6.4 Hz, 2H), 2.21-2.11 (m, 2H), 1.91-1.87 (m, 3H), 1.66-1.63 (m, 2H), 1.46 (s, 9H), 1.33-1.28 (m, 10H), 0.92 (t, 3H) ppm. ¹³C NMR (CDCl₃, 125 MHz, mixture of rotamers), δ: 199.4, 155.2, 148.8, 134.5, 129.5, 128.6, 128.2, 79.6, 70.0, 55.6, 54.7, 40.3, 36.0, 31.9, 31.1, 29.7, 29.6, 29.4, 29.3, 29.2, 28.5, 22.7, 14.1, 14.1 ppm. HRMS (ESI) calcd. for C₂₅H₄₀NO₃ (M+H)⁺ 454.29280, found 454.29428.

(2R,4R)-4-Hydroxy-2-(3-(4-octylphenyl)-3-oxopropyl)pyrrolidin-1-ium chloride (15): 15 was synthesized in accordance with the general procedure A (43 mg, 0.10 mmol). 15 was obtained as a white solid (26 mg, 72%). As for 13, 15 revealed prone to cyclize spontaneously to 15c in protic solvent such as MeOH. IR (neat), vₘₐₓ: 2977, 2926, 2855, 1701, 1396, 1260, 987, 753 cm⁻¹. ¹H NMR (CDCl₃, 500 MHz, opened form), δ: 7.96-7.94 (d, J=8.3 Hz, 2H), 7.35-7.33 (d, J=8.3 Hz, 2H), 4.54 (m, 1H), 3.95-3.90 (m, 1H), 3.48-3.43 (dd, J=12.5, 4.1 Hz, 1H), 3.28-3.19 (m, 3H), 2.71-2.67 (m, 2H), 2.26-2.10 (m, 3H), 1.87-1.80 (m, 1H), 1.65 (m, 2H), 1.34-1.29 (m, 10H), 0.91-0.88 (t, J=6.85 Hz, 3H) ppm. ¹H NMR (CDCl3, 500 MHz, cyclized form), δ: 8.00-7.98 (d, J=8.5Hz, 1H), 7.59-7.57 (d, J=8.4 Hz, 2H), 5.24 (m, 1H), 4.93-4.90 (m, 1H), 4.33-4.29 (m, 1H), 4.17-4.13 (m, 2H), 3.74 (m, 1H), 2.83-2.79 (m, 2H), 2.64 (m, 1H), 2.36-2.32 (dd, J=13.1, 6.1 Hz, 1H), 2.12-2.09 (m, 1H), 1.97-1.89 (m, 1H), 1.71 (m, 2H), 1.36-1.31 (m, 10H), 0.93-0.90 (t, J=6.9 Hz, 3H) ppm. HRMS (ESI) calcd. for C₂₅H₄₀NO₃ (M+H)⁺ 618.35301, found 618.35381.

(2S)-tert-Butyl 2-((4-bromophenyl)(hydroxy)methyl)pyrrolidine-1-carboxylate (17a1): BuLi (2.5 M in hexane, 11.4 mL, 24 mmol, 2.0 eq.) was added dropwise via syringe to a stirred and cooled to −78° C. solution of 1,4-dibromobenzene (5.88 g, 24.9 mmol, 2.06 eq.) in dry THF (42 mL) under Ar. Stirring was continued for 30 min before N-Boc-D-prolinal 17a (2.2038 g, 1.0 mmol) in THF (18 mL) was added via syringe over ca 5 min. Stirring was continued for a further 15 min and the mixture was quenched by addition of sat. NH₄Cl (20 mL) and H₂O (10 mL). The bulk of the THF was then removed in vacuo and the mixture was extracted into Et₂O (30 mL), washed once with brine (15 mL) and dried (MgSO₄). Evaporation of the solvent and chromatography over SiO₂ (2.5×40 cm) using 13% EtOAc-hexanes afforded (2S)-tert-butyl 2-((4-bromophenyl) (hydroxy)methyl) pyrrolidine-1-carboxylate 17a1 as a mixture of diastereomers (2.3 g, 59%).

(S)-tert-Butyl 2-(4-bromobenzoyl)pyrrolidine-1-carboxylate (17b): Dess-Martin periodinane (3.5 g, 8.2 mmol, 1.3 eq.) was added as a solid over ca 2 min to a stirred and cooled (0° C.) solution of (2S)-tert-butyl 2-((4-bromophenyl) (hydroxy)methyl)pyrrolidine-1-carboxylate 17a1 (mixture of diastereomers, 2.3 g, 6.5 mmol, 1.0 eq.) in CH₂Cl₂ (24 mL). The flask was capped with a glass stopper and stirring was continued overnight.

The mixture was then quenched by the addition of sat. NaHCO₃ (10 mL) and H₂O (5 mL) and stirring was continued for 30 min. The mixture was then filtered through Celite (2×3 cm), washing the filter cake with CH₂Cl₂. The aqueous layer was extracted once with CH₂Cl₂ (10 mL) and the combined organic was dried (MgSO₄), evaporated, and chromatographed over SiO₂ (2.5×30 cm) using 13% EtOAc-hexanes to afford (S)-tert-butyl 2-(4-bromobenzoyl)pyrrolidine-1-carboxylate 17b (1.6011 g, 70%). HRMS (ESI) calcd. for C₁₆H₂₀BrNO₃ (M+Na)⁺ 376.05188, found 376.05202.

tert-Butyl(S)-2-((R)-1-(4-bromophenyl)-3-ethoxy-1-hydroxy-3-oxopropyl)pyrrolidine-1-carboxylate (17c) and tert-butyl (S)-2-((S)-1-(4-bromophenyl)-3-ethoxy-1-hydroxy-3-oxopropyl)pyrrolidine-1-carboxylate (epi-17c): BuLi (2.5 M in hexane, 5.5 mL, 13.7 mmol, 3.0 eq.) was added via syringe to a stirred and cooled to −78° C. solution of i-Pr₂NH (1.9 mL, 13.6 mmol, 3.0 eq.) in THF (12 mL). The cooling bath was removed for 10 min and then replaced and stirring was continued for a further 10 min before EtOAc (1.5 mL, 15.4 mmol, 3.4 eq.) was added dropwise via syringe. Stirring was then continued for 45 min before (S)-tert-butyl-2-(4-bromobenzoyl)pyrrolidine-1-carboxylate 17b (1.6011 g, 4.55 mmol, 1.0 eq.) in THF (5 mL+1 mL rinse) was added at a slow dropwise rate via syringe (ca 15 min). Stirring was then continued for 20 min and then the mixture was quenched by the addition of sat. NH₄Cl (5 mL) and H₂O (5 mL). The mixture was diluted with Et₂O (30 mL) and washed once with H₂O (20 mL), once with brine (20 mL) and dried (Na₂SO₄). Evaporation of the solvent provided (S)-tert-butyl 2-((S)-1-(4-bromophenyI)-3-ethoxy-1-hydroxy-3-oxopropyl)pyrrolidine-1-carboxylate 17c and (S)-tert-butyl 2-((R)-1-(4-bromophenyI)-3-ethoxy-1-hydroxy-3-oxopropyl)pyrrolidine-1-carboxylate epi-17c as a mixture (1.54 g, 76%), which was used directly in the next step without further purification. HRMS (ESI) calcd. for C₂₅H₄₀NO₃ (M+H)⁺ 464.10431, found 464.10432.

tert-Butyl(S)-2-((R)-1-(4-bromophenyl)-1,3-dihydroxypropyl)pyrrolidine-1-carboxylate (17c1) and tert-butyl(S)-2-((S)-1-(4-bromophenyl)-1,3-dihydroxypropyl)pyrrolidine-1-carboxylate (epi-17c1): LiBH₄ solution (2 M in THF, 1.1 mL, 2.2 mmol, 0.6 eq.) was added via syringe to a stirred and cooled to 0° C. solution of esters (S)-tert-butyl 2-((S)-1-(4-bromophenyl)-3-ethoxy-1-hydroxy-3-oxopropyl)pyrrolidine-1-carboxylate 17c and (S)-tert-butyl 2-((R)-1-(4-bromophenyl)-3-ethoxy-1-hydroxy-3-oxypropyl)pyrrolidine-1-carboxylate epi-17c (mixture from previous step, 1.54 g, 3.48 mmol, 1.0 eq.) in THF (10 mL) under Ar. The ice-bath was left in place but not recharged and stirring was continued for 7 h. The mixture was then quenched by the careful addition of H₂O (3 mL) and then NaHCO_{3aq}(sat., 5 mL). EtOAc (10 mL) was then added and the biphasic mixture was stirred for 1 h. The aqueous phase was extracted once with EtOAc (10 mL) and the combined organic was washed once with brine (10 mL) and dried (Na₂SO₄). Evaporation of the solvent and filtration of the residue through a plug of SiO₂ (2×4 cm) using 40% EtOAc-hexanes (ca 100 mL, TLC control) afforded the alcohols (S)-tert-butyl 2-((R)-1-(4-bromophenyl)-1,3-dihydroxypropyl)pyrrolidine-1-carboxylate 17c1 and (S)-tert-butyl 2-((S)-1-(4-bromophenyl)-1,3-dihydroxypropyl)pyrrolidine-1-carboxylate epi-17c1 as a mixture (1.38 g, 72%).

tert-Butyl(S)-2-((R)-1-(4-bromophenyl)-1-hydroxy-3-(tosyloxy)propyl)pyrrolidine-1-carboxylate (17d) and (S)-tert-butyl 2-((S)-1-(4-bromophenyl)-1-hydroxy-3-(tosyloxy)propyl)pyrrolidine-1-carboxylate (epi-17d): Et₃N (0.93 mL, 6.7 mmol, 1.2 eq.) followed by TsCl (1.16 g, 6.1 mmol, 1.1 eq.) were added to a stirred solution of alcohols (S)-tert-butyl-2-((R)-1-(4-bromophenyl)-1,3-dihydroxypropyl) pyrrolidine -1-carboxylate 17c1 and (S)-tert-butyl-2-((S)-1-(4-bromophenyl)-1,3-dihydroxypropyl) pyrrolidine-1-carboxylate epi-17c1 (mixture from previous step, 2.22 g, 5.55 mmol, 1.0 eq.) in CH₂Cl₂ (10 mL). The flask was capped with a glass stopper and stirred for 8 h. The mixture was diluted with CH₂Cl₂ (10 mL), washed once with H₂O (25 mL), and dried (MgSO₄). Removal of the solvent in vacuo and chromatography over SiO₂ (2.5×35 cm) using 10% EtOAc-hexanes afforded the tosylates (S)-tert-butyl 2-((R)-1-(4-bromophenyl)-1-hydroxy-3-(tosyloxy)propyl)pyrrolidine-1-carboxylate 17d and (S)-tert-butyl 2-((S)-1-(4-bromophenyl)-1-hydroxy-3-(tosyloxy)propyl)pyrrolidine-1-carboxylate epi-17d as a mixture (2.23 g, 72%).

(1R)-1-(4-Bromophenyl)-1-hydroxyoctahydropyrrolizin-4-ium 4-methylbenzenesulfonate (17e) and (1S)-1-(4-bromophenyl)-1-hydroxyoctahydropyrrolizin-4-ium 4-methylbenzenesulfonate (epi-17e): A solution of tosylates (S)-tert-butyl 2-((R)-1-(4-bromophenyl)-1-hydroxy-3-(tosyloxy)propyl)pyrrolidine-1-carboxylate 17d and (S)-tert-butyl 2-((S)-1-(4-bromophenyl)-1-hydroxy-3-(tosyloxy)propyl)pyrrolidine-1-carboxylate epi-17d (2.2 g, 4.0 mmol, 1.0 eq.) was stirred for 10 h at 110° C. in PhMe (18 mL) under Ar. The solvent was then removed in vacuo and the residue was chromatographed over SiO₂ (2.5×35 cm) using 10-20% MeOH-CHCl₃ to give a faster eluting fraction ((1R,7aS)-1-(4-bromophenyl)-1-hydroxyoctahydropyrrolizin-4-ium 4-methylbenzenesulfonate) 17e and a slower eluting fraction ((1S,7aS)-1-(4-bromophenyl)-1-hydroxyoctahydropyrrolizin-4-ium 4-methylbenzenesulfonate) epi-17e. Mixed fractions were discarded. After removal of the solvent, the faster eluting diastereomer was crystallized from CH₂Cl₂-t-BuOMe (note 1) to provide ((1R,7aS)-1-(4-bromophenyl)-1-hydroxyoctahydropyrrolizin-4-ium 4-methylbenzenesulfonate 17e (0.7 g, 38%). The slower eluting fraction was also evaporated to dryness and the solid residue was suspended in CH₂Cl₂ (3 mL) and filtered through a syringe filter (25 mm, PTFE 0.45 μm) to remove silica washing with three portions of CH₂Cl₂ (3 ml each) (note 2). The solvent was then removed in vacuo and the solid residue was crystallized from CH₂Cl₂-t-BuOMe (note 3) to provide (1S,7aS)-1-(4-bromophenyl)-1-hydroxyoctahydropyrrolizin-4-ium 4-methylbenzenesulfonate epi-17e (0.3 g, 17%). 17e: mp 143° C. α²⁵_{D}⁺ 21 (c 0.65, MeOH). IR (neat), vₘₐₓ: 3371, 3673, 1657, 1394, 561 cm⁻¹. ¹H NMR (400 MHz, CDCl₃), δ: 1.74-1.83 (m, 1H), 2.05-2.13 (m, 1H), 2.24-2.33 (m, 2H), 2.39 (s, 3H), 2.45 (dd, J=5.4, 13.4 Hz, 1H), 2.81 (ddd, J=7.2, 13.0, 13.0 Hz, 1H), 3.03-3.09 (m, 1H), 3.32 (ddd, J=5.9, 12.8, 12.8 Hz, 1H), 3.55 (ddd, J=6.0, 6.0, 11.7 Hz, 1H), 3.80-3.87 (br s, 1H), 3.94-3.99 (m, 1H), 4.38 (app dd, J=4.5, 8.0 Hz, 1H), 7.18 (d, J=8.1 Hz, 2H), 7.36 (d, J=8.7 Hz, 2H), 7.45 (d, J=8.7 Hz, 2H), 7.71 (d, J=8.1 Hz, 2H), 11.26 (br s, 1H) ppm. ¹³C NMR (100 MHz, CDCl₃), δ: 21.8, 22.8, 27.5, 42.6, 53.7, 55.7, 79.4, 122.3, 126.1, 127.7, 129.4, 132.0, 140.3, 141.0, 142.0 ppm. LRMS found m/z 282.0. epi-17e: mp=177.5-178.5° C. IR (neat), vₘₐₓ: 1234, 1185, 1009, 815, 696, 568, 478 cm⁻¹. ¹H NMR (400 MHz, CDCl₃), δ: 1.18-1.26 (m, 1H), 1.77-1.84 (m, 1H), 1.93-2.01 (m, 2H), 2.39 (s, 3H), 2.53-2.66 (overlapping m, 2H), 2.97 (ddd, J=11.4, 9.6, 6.8 Hz, 1H), 3.26 (dd, J=11.6, 6.4 Hz, 1H), 3.98 (ddd, J=11.3, 6.1, 6.1 Hz, 1H), 4.31 (ddd, J=11.9, 11.9, 7.1 Hz, 1H), 4.61 (dd, J=10.0, 8.2 Hz, 1H), 5.80 (br s, 1H), 7.18 (d, J=7.9 Hz, 2H), 7.33 (d, J=8.6 Hz, 2H), 7.49 (d, J=8.6 Hz, 2H), 7.73 (d, J=7.9 Hz, 2H) ppm. ¹³C NMR (100 MHz, CDCl₃), δ: 4.3, 21.8, 26.0, 29.8, 33.5, 54.2, 57.2, 81.8, 123.1, 126.3, 128.6, 129.3, 132.2, 139.4, 140.9, 141.8 ppm. LRMS found m/z 282.0. Note 1: Diastereomer (1R,7aS)-1-(4-bromophenyl)-1-hydroxyoctahydropyrrolizin-4-ium 4-methylbenzenesulfonate was dissolved in ca. 10 mL of CH₂Cl₂ and was then brought to boiling with a heat gun. t-BuOMe (ca 5 mL) was then added and the solution was allowed to crystallize overnight, the flask being left completely open. The next day the flask was capped with a glass stopper and cooled at ca −15° C. (freezer section of fridge) for a further 10 h. Filtration afforded flocculent white needles of ((1R,7aS)-1-(4-bromophenyl)-1-hydroxyoctahydropyrrolizin-4-ium 4-methylbenzenesulfonate. Note 2: The syringe filter was attached to a syringe and the plunger was removed. The suspension was then transferred to this syringe via Pasteur pipette and forced through the filter by re-inserting the plunger. Note 3: Diastereomer (1S,7aS)-1-(4-bromophenyl)-1-hydroxyoctahydropyrrolizin-4-ium 4-methylbenzenesulfonate was dissolved in ca 10 mL of CH₂Cl₂ with stirring in an oil bath set at 45° C. tBu-OMe was added (ca 3mL) and stirring was discontinued. The oil bath was shut off but left in place and the mixture was allowed to crystallize overnight, the flask being left completely open. The next day the flask was capped with a glass stopper and cooled at ca −15° C. (freezer section of fridge) for a further 10 h. Filtration afforded small needles.

(1R)-1-(4-Octylphenyl)hexahydro-1H-pyrrolizin-1-ol hydrochloride (17): To a solution of catecholborane (135 μL, 1.0 M in THF, 0.135 mmol, 1.5 eq.) was added the octyne dropwise (19.9 μL, 0.135 mmol, 1.5 eq.). Gas formation was observed during the addition. The colorless solution was refluxed for 2h then cooled back to rt. In another flask, 17e was dissolved in a biphasic mixture of DME (1.1 mL) and aqueous NaHCO₃ (1 M) solution, then the octyne/catecholborane solution was syringed into the flask. Pd(PPh3)4 (3.1 mg, 0.0027 mmol, 0.03 eq.) was added and the overall white suspension was refluxed overnight. Et₂O and brine added. The aqueous layer was extracted ×2 with Et₂O. The organic layers were collected, dried over Na₂SO₄, filtered through Celite, concentrated. The crude oil was chromatographed over SiO₂ (9:1 DCM/MeOH) to deliver an orange oil (18 mg). The product was engaged in the next step without further purification. The previously obtained oil (12 mg, 0.038 mmol, 1.0 eq.) was dissolved in MeOH (1.2 mL) and Pd/C was added in one portion (0.4 mg, 0.0038 mmol, 0.1 eq.). The flask was purged ×3 with H₂ and the black suspension was stirred for 1 h 30. Then HCl (9.5 μL, 4 M, 0.038 mmol, 1.0 eq.) was added and the solution was stirred for 2 h, then filtered through a pad of Celite and concentrated. The resulting crude was chromatographed on reversed C18 column (0 to 20% MeCN in H₂O) to deliver 17 as a colorless oil (9 mg, 67%). IR (neat), vₘₐₓ: 3357, 2923, 1593, 1349 cm⁻¹. ¹H NMR (CDCl3, 500 MHz, mixture of rotamers), δ: 7.14-7.08 (m, 4H), 4.78 (bs, 1H), 4.60-4.57 (t, J=, 0.47H), 4.54-4.50 (t, J=14.8, 0.53H), 3.91-3.67 (m, 3H), 3.54-3.47 (m, 1H), 2.58-2.54 (m, 2H), 2.30-2.25 (m, 0.53H), 2.13-2.09 (m, 0.47H), 1.84-1.81 (m, 1H), 1.58-1.56 (m, 2H), 1.58-1.40 (m, 25H), 1.29-1.25 (m, 13H), 0.89-0.86 (t, J=, 3H) ppm. ¹³C NMR (CDCl₃, 125 MHz, mixture of rotamers), δ: 207.1, 207.1, 154.6, 153.8, 142.2, 141.9, 130.6, 130.3, 129.7, 129.6, 129.0, 128.9, 83.1, 83.1, 83.1, 83.0, 82.9, 80.9, 80.5, 75.7, 75.6, 74.8, 74.8, 63.4, 62.5, 53.8, 53.7, 53.5, 53.5, 47.6, 46.3, 32.0, 30.0, 30.0, 29.6, 29.4, 28.5, 28.4, 22.8, 14.2 ppm. HRMS (ESI) calcd. for C₂₃H₃₇NO₃Na (M+H)⁺ 316.2635, found 316.2644.

tert-Butyl 3-hydroxy-3-(4-octylphenyl)pyrrolidine-1-carboxylate ((±)18b): (±)18b was synthesized in accordance with the general procedure C (100 mg, 0.54 mmol). The resulting residue was purified by flash column chromatography (hexane/EtOAc 8:2, Rf:0.28) to give (±)18b as a pale yellow oil (123 mg, 61%). IR (neat), vₘₐₓ: 3392, 2923, 1670, 1412, 1134 cm⁻¹. ¹H NMR (500 MHz, CDCl₃, mixture of rotamers) δ 7.37 (t, J=6.6 Hz, 2H), 7.18 (d, J=8.0 Hz, 2H), 3.80-3.49 (m, 4H), 2.65-2.50 (m, 2H), 2.36-2.09 (m, 2H), 1.67-1.54 (m, 2H), 1.47 (d, J=11.1 Hz, 9H), 1.38-1.11 (m, 10H), 0.88 (t, J=7.0 Hz, 3H). ¹³C NMR (125 MHz, CDCl₃, mixture of rotamers) δ 154.9, 154.8, 142.8, 140.1, 128.7, 125.2, 125.2, 80.7, 79.9, 79.6, 59.7, 58.8, 45.2, 44.7, 39.6, 38.9, 35.7, 32.0, 31.6, 29.9, 29.6, 29.5, 29.4, 28.7, 22.8, 14.3. HRMS (ESI) calcd. for C₂₃H₃₇NO₃Na (M+Na)⁺ 398.2666, found 398.2681.

3-Hydroxy-3-(4-octylphenyl)pyrrolidin-1-ium chloride ((±)18): 18 was synthesized in accordance with the general procedure XX (25 mg, 0.066 mmol). The resulting residue was triturated with a mixture of CH₂Cl₂/Et₂O (9:1) to give 18 as a pale yellow oil (9.6 mg, 45%, Rf:0.18 CH₂Cl₂/MeOH 9:1, 1% Et₃N). IR (neat), vₘₐₓ: 3385, 2922, 1617, 1379, 1179, 1086 cm⁻¹. ¹H NMR (500 MHz, MeOD) δ 7.44 (d, J=8.1 Hz, 2H), 7.22 (d, J=8.0 Hz, 2H), 3.61 (d, J=9.8 Hz, 2H), 3.43 (dd, J=30.3, 11.5Hz, 2H), 2.68-2.55 (m, 2H), 2.43 (dd, J=23.4, 10.3 Hz, 1H), 2.33 (d, J=11.0 Hz, 1H), 1.61 (d, J=7.3 Hz, 2H), 1.40-1.22 (m, 10H), 0.89 (t, J=6.9 Hz, 3H). ¹³C NMR (125 MHz, MeOD) δ 142.7, 137.9, 128.3, 125.1, 79.6, 56.6, 48.1, 48.0, 47.8, 47.6, 47.4, 47.3, 47.1, 44.4, 38.1, 35.0, 31.6, 31.3, 29.2, 29.0, 28.9, 22.3, 13.0. HRMS (ESI) calcd. for C₁₈H₂₉NO (M+H)⁺ 276.2322, found 276.2326.

tert-Butyl (S)-2-(methoxy(methyl)carbamoyl)pyrrolidine-1-carboxylate (19b): To a −20° C. cooled solution containing 19a (321 mg, 1.4 mmol, 1.0 eq.) and N,O-dimethylhydroxylamine-HCl (205 mg, 2.10 mmol, 1.5 eq.) in dry THF (1 mL) was added iPrMgCl (1.4 mL, 2.8 mmol, 2.0 M in THF, 2.0 eq.) dropwise. The light brown solution was stirred at −10° C. for 20 min whereby additional N,O-dimethylhydroxylamine-HCl (205 mg, 2.10 mmol, 1.5 eq.) was added in one portion followed by iPrMgCl (1.4 mL, 2.8 mmol, 2.0 M in THF, 2.0 eq.) dropwise. The reaction was stirred for 20 additional minutes at −10° C. whereby TLC analysis indicated that the reaction had gone to completion. Saturated aqueous NH₄Cl solution (5 mL) was added and the resulting aqueous layer was extracted with EtOAc (2×5 mL). The organic layers were collected, dried over Na₂SO₄, filtered, concentrated to give 19b as a pure incolore oil (321 mg, 89%) which was brought to the next step without further purification (Rf: 0.34 hexanes/EtOAc 5:5). α²⁰_{D} −13.92 (c 1.25, CHCl₃). The spectral datas matched those reported in the litterature.⁶

tert-Butyl (S)-2-(4-octylbenzoyl)pyrrolidine-1-carboxylate (19c): 19c was synthesized in accordance with the general procedure C (100 mg, 0.39 mmol). The resulting residue was purified by flash column chromatography (hexane/EtOAc 8:2 Rf 0.32) to give 19c as a yellow oil (98 mg, 65%). α²⁰_{D} −78.05 (c 0.21, CHCl₃). IR (neat), vₘₐₓ: 2925, 2584, 1687, 1391, 1160 cm⁻¹. ¹H NMR (500 MHz, CDCl₃) δ 7.88 (dd, J=19.0, 8.2 Hz, 2H), 7.25 (dd, J=15.6, 7.8 Hz, 2H), 5.37-5.16 (m, 1H), 3.76-3.41 (m, 2H), 2.64 (dt, J=11.2, 7.8 Hz, 2H), 2.37-2.22 (m, 1H), 1.99-1.85 (m, 3H), 1.67-1.55 (m, 2H), 1.46 (s, 4H), 1.36-1.20 (m, 16H), 0.87 (t, J=7.0 Hz, 3H). ¹³C NMR (CDCl₃, 125 MHz, mixture of rotamers), δ: 198.7, 198.2, 154.6, 154.0, 149.1, 149.1, 133.0, 132.9, 128.8, 128.8, 128.8, 128.4, 79.8, 79.7, 61.4, 61.1, 46.9, 46.8, 36.1, 32.0, 31.2, 29.5, 29.4, 29.3, 28.6, 28.3, 23.7, 22.8, 14.2 ppm. HRMS (ESI) calcd. for C₂₄H₃₇NO₃ (M+Na)⁺ 410.26660, found 410.26710.

(S)-2-(4-Octylbenzoyl)pyrrolidin-1-ium chloride (19): 19 was synthesized in accordance with the general procedure A (27 mg, 0.07 mmol). The resulting residue was triturated with EtOAc to give 19 as a white powder (14 mg, 64%). α²⁰_{D} −38.71 (c 0.16, CHCl₃). IR (neat), vₘₐₓ: 2923, 2853, 1686, 1397, 1250, 997 cm⁻¹. ¹H NMR (500 MHz, MeOD) δ 8.00 (d, J=8.3 Hz, 2H), 7.43 (d, J=8.3 Hz, 2H), 5.34 (dd, J=9.3, 7.1 Hz, 1H), 3.45 (dt, J=15.3, 6.1 Hz, 2H), 2.79-2.61 (m, 1H), 2.24-1.90 (m, 2H), 1.73-1.58 (m, 2H), 1.42-1.23 (m, 10H), 0.90 (t, J=7.0 Hz, 3H). ¹³C NMR (CDCl₃, 125 MHz, mixture of rotamers), δ: 194.9, 152.9, 132.1, 130.8, 130.7, 64.8, 47.8, 37.3, 33.3, 32.6, 31.4, 30.9, 30.7, 30.6, 25.5, 24.0, 14.7 ppm. HRMS (ESI) calcd. for C₁₉H₃₀NO (M+H)⁺ 288.23219, found 288.23192.

tert-Butyl(2S,4R)-4-((tert-butyldimethylsilyl)oxy)-2-(methoxy(methyl)carbamoyl)pyrrolidine-1-carboxylate (20b): 20a was synthesized in accordance with the procedure from 19b (50.0 mg, 0.14 mmol). The crude incolore oil of 20b (54 mg, 95%) was brought to the next step without further purification (Rf: 0.25 hexanes/EtOAc 7:3). α²⁰_{D} −14.00 (c 0.50, CHCl3).

tert-Butyl(2R,4S)-4-((tert-butyldimethylsilyl)oxy)-2-(methoxy(methyl)carbamoyl)pyrrolidine-1-carboxylate (21b): 21b was synthesized in accordance with the procedure of its enantiomer 20b (300 mg, 0.84 mmol). 21b was obtained as a colorless oil (298 mg, 91%) which was brought to the next step without further purification (Rf:0.25 hexanes/EtOAc 7:3). α²⁰_{D}⁺ 13.00 (c 1.00, CHCl₃). The spectral datas matched those reported for its enantiomer.

tert-Butyl (2S,4R)-4-hydroxy-2-(4-octylbenzoyl)pyrrolidine-1-carboxylate (20c): 20b1 was synthesized in accordance with the general procedure C (200 mg, 0.52 mmol). 20b1 was obtained as a yellow oil which was submitted to general procedure B without further purification. The resulting residue was purified by flash column chromatography (hexane/EtOAc 4:6 Rf. 0.35) to give 20c as a yellow oil (135 mg, 65% over 2 steps). α²⁰_{D}−6.66 (c 0.27, CHCl₃). IR (neat), vₘₐₓ: 2924, 1686, 1605, 1399, 1158 cm⁻¹. ¹H NMR (CDCl₃, 500 MHz, mixture of rotamers), δ: 7.95-7.90 (m, 2H), 7.31-7.27 (m, 2H), 5.51-5.48 (t, J=7.6 Hz, 0.45 H), 5.42-5.39 (t, J=8.1 Hz, 0.55 H), 4.55 (s, 1H), 3.80-3.56 (m, 2H), 2.71-2.68 (m, 2H), 2.41-2.37 (m, 1H), 2.09-2.04 (m, 1H), 1.74-1.62 (m, 3H), 1.49 (s, 3H), 1.32-1.26 (m, 15 H), 0.92-0.89 (t, J=7.0 Hz, 3H) ppm. ¹³C NMR (CDCl₃, 125 MHz, mixture of rotamers), δ: 199.0, 198.5, 154.9, 154.4, 149.7, 149.6, 133.4, 133.3, 129.2, 129.1, 129.1, 128.7, 80.7, 80.4, 71.0, 70.3, 60.0, 59.7, 55.6, 40.1, 39.3, 36.4, 32.2, 31.5, 31.4, 29.8, 29.6, 29.6, 28.8, 28.5, 23.0, 14.5 ppm. HRMS (ESI) calcd. for C₂₅H₄₀NO₃ (M+H)⁺ 426.26150, found 426.26245.

tert-Butyl (2R,4S)-4-hydroxy-2-(4-octylbenzoyl)pyrrolidine-1-carboxylate (21c): 21b1 was synthesized in accordance with the general procedure C (298 mg, 0.77 mmol). 21b1 was obtained as a yellow oil which was submitted to general procedure B without further purification. The resulting residue was purified by flash column chromatography (hexane/EtOAc 4:6 Rf 0.35) to give 21c as a yellow oil (205 mg, 62% over 2 steps). α²⁰_{D}+38.18 (c 1.65, CHCl₃). The spectral datas matched those reported for its enantiomer.

(2S,4R)-4-Hydroxy-2-(4-octylbenzoyl)pyrrolidin-1-ium chloride (20): 20 was synthesized in accordance with the general procedure A (18 mg, 0.07 mmol). 20 was obtained as a white solid (18 mg, 86%). α²⁰_{D} −30.69 (c 0.80, CHCl3). IR (neat), vₘₐₓ: 2923, 2470, 2070, 1596, 1463, 1119, 973 cm⁻¹. ¹H NMR (CDCl3, 500 MHz), δ: 7.99-7.98 (d, J=8.2 Hz, 1H), 7.43-7.42 (d, J=7.9 Hz, 2H), 5.51-5.47 (dd, J=10.3, 8.2 Hz, 1H), 4.61-4.60 (m, 1H), 3.39 (s, 2H), 2.73-2.71 (m, 2H), 2.66-2.62 (dd, J=12.9, 8.2 Hz, 1H), 2.07-2.02 (ddd, J=13.8, 10.4, 4.2 Hz, 1H), 1.68-1.65 (m, 2H), 1.34-1.28 (m, 10H), 0.91-0.88 (t, J=7.0 Hz, 3H) ppm. ¹³C NMR (CDCl₃, 125 MHz), δ: 194.6, 162.8, 152.6, 131.8, 130.4, 130.4, 71.3, 63.3, 55.0, 40.5, 37.0, 33.0, 32.2, 30.5, 30.4, 30.3, 23.7, 14.4 ppm. HRMS (ESI) calcd. for C₂₅H₄₀NO₃ (M+H)⁺ 304.22770, found 304.22860.

(2R,4S)-4-Hydroxy-2-(4-octylbenzoyl)pyrrolidin-1-ium chloride (21): 21 was synthesized in accordance with the general procedure A (30 mg, 0.07 mmol). 21 was obtained as a white solid (16 mg, 64%). α²⁰_{D} 25.63 (c 0.34, CHCl₃). The spectral datas matched those reported for its enantiomer.

tert-Butyl (2R,4R)-4-hydroxy-2-(4-octylbenzyl)pyrrolidine-1-carboxylate (16a): 20c (20 mg, 0.050 mmol) was dissolved in EtOH (5 mL) and Pd/C (10%, 24 mg) was added to the resulting solution. The air was removed from the flask under vacuum and replaced with hydrogen (balloon). The reaction was vigorously stirred for 24 hours at room temperature. Afterwards, the mixture was filtered through a celite pad, washing with abundant EtOH, and the collected solution was concentrated in vacuo. The crude was purified by flash column chromatography (EtOAc/hexane 1:1, Rf: 0.35) to give 16a as a colorless oil (6 mg, 32%). α²⁵_{D} −32.7 (c 0.30, CHCl3). IR (neat), vₘₐₓ: 3408, 2923, 2853, 1694, 1668, 1513, 1455, 1393, 1365, 1253, 1153, 1116, 981, 858, 770, 553 cm⁻¹.¹H NMR (CDCl₃, 500 MHz, mixture of rotamers), δ: 7.11-7.04 (m, 4H), 4.22-4.14 (m, 2H), 3.50 (br. s, 0.6 H), 3.35 (br. s, 0.4 H), 3.30 (br. s, 1H), 3.09 (br. s, 1H), 2.68 (br. s, 0.4 H), 2.63 (br. s, 0.6 H), 2.57-2.54 (m, 2H), 1.87 (br. s, 2H), 1.60-1.55 (m, 2H), 1.52 (s, 9H), 1.31-1.26 (m, 10H), 0.87 (t, J=7.0 Hz, 3H) ppm. ¹³C NMR (CDCl₃, 125 MHz, mixture of rotamers), δ: 154.9, 141.0, 135.3, 129.3, 128.4, 79.7, 69.7, 69.4, 57.3, 54.5, 40.3, 39.4, 35.6, 31.9, 31.6, 29.5, 29.3, 29.2, 28.6, 22.6, 14.1 ppm. HRMS (ESI) calcd. for C₂₄H₃₉NO₃Na (M+Na)⁺ 412.28222, found 412.28110.

(3R,5R)-5-(4-Octylbenzyl)pyrrolidin-3-ol (16): Prepared according to general procedure A, starting from 16a (6 mg, 0.015 mmol). The crude was triturated in Et₂O to give product 16 as a white solid (5 mg, 100%). For biological testing a portion of the product was dissolved in the minimum amount of HPLC grade water, filtered (pore size=0.45 μm) and lyophilized. α_{D +}4.0 (c 0.25, MeOH). IR (neat), vₘₐₓ: 3318, 2920, 2851, 1515, 1437, 1394, 1314, 1266, 1159, 1080, 1063, 1031, 961, 772, 720, 615, 531, 450 cm⁻¹. ¹H NMR (CD₃OD, 500 MHz), δ: 7.25 (d, J=8.1 Hz, 2H), 7.21 (d, J=8.1 Hz, 2H), 4.56 (t, J=4.2 Hz, 1H), 4.12-4.05 (m, 1H), 3.50 (dd, J=12.4, 4.2 Hz, 1H), 3.19 (d, J=12.4 Hz, 1H), 3.05 (d, J=7.6 Hz, 2H), 2.63-2.60 (m, 2H), 2.13 (dd, J=13.7, 5.8 Hz, 1H), 1.90 (ddd, J=13.7, 11.6, 4.2 Hz, 1H), 1.65-1.59 (m, 2H), 1.34-1.31 (m, 10H), 0.92 (t, J=7.0 Hz, 3H) ppm. ¹³C NMR (CD₃OD, 125 MHz), δ: 141.9, 133.6, 128.7, 128.4, 69.0, 60.3, 53.0, 39.5, 37.1, 35.1, 31.6, 31.3, 29.2, 29.0, 28.9, 22.3, 13.0 ppm.

tert-Butyl(2R,4S)-4-(((tert-butyldiphenylsilyl)oxy)methyl)-2-(methoxy(methyl)carbamoyl)pyrrolidine-1-carboxylate (22b): Isopropyl magnesium chloride (585 □L, 2 M in THF, 1.17 mmol, 6 eq.) was added dropwise to a solution of 13a (100 mg, 0.195 mmol) and N,O-dimethylhydroxylamine (87 mg, 0.89 mmol, 4.5 eq.) in dry THF (1 mL) at −20° C. The resulting mixture was kept at the same temperature and stirred for 1 h. Afterwards, the reaction was quenched at −20° C. by adding NH₄Cl satd. sol. (5 mL) and the product was extracted with EtOAc (3×5 mL). The organic layers were washed with brine (5 mL), dried over Na₂SO₄, filtered and concentrated. The residue was purified by flash column chromatography (EtOAc/hexane 1:2, Rf: 0.09) to give 22b as a colorless oil (102 mg, 99%). α_{D}⁺ 13.6 (c 1.7, CHCl3). IR (neat), vₘₐₓ: 2931, 2858, 1696, 1472, 1427, 1388, 1365, 1319, 1256, 1164, 1109, 999, 940, 909, 879, 823, 741, 702, 613, 504, 489 cm⁻¹. ¹H NMR (CDCl3, 500 MHz, mixture of rotamers), δ: 7.65-7.63 (m, 4H), 7.43-7.37 (m, 6H), 4.75 (d, J=6.5, 0.5H), 4.65 (d, J=6.2, 0.5H), 3.80-3.78 (m, 0.5H), 3.77 (s, 1.5H), 3.70 (s, 1.5H), 3.69-3.65 (m, 0.5H), 3.59 (dd, J=6.1, 1.7 Hz, 2H), 3.35 (dd, J=10.5, 7.0 Hz, 0.5H), 3.29 (dd, J=10.5, 7.1 Hz, 0.5H), 3.20 (s, 3H), 2.69-2.55 (m, 1H), 2.11 (dt, J=12.9, 8.8 Hz, 0.5H), 2.03 (dt, J=12.9, 9.5Hz, 0.5H), 1.95-.1.90 (m, 1H), 1.46 (s, 4.5H), 1.42 (s, 4.5H), 1.05 (s, 4.5H), 1.04 (s, 4.5H) ppm. ¹³C NMR (CDCl₃, 125 MHz, mixture of rotamers), δ: 173.1, 154.5, 153.8, 135.5, 133.4, 133.3, 129.6, 127.7, 79.5, 79.4, 65.0, 61.3, 61.2, 56.7, 56.4, 49.3, 49.1, 39.5, 38.6, 32.7, 32.0, 28.5, 28.4, 26.8, 26.7, 19.2 ppm. HRMS (ESI) calcd. for C₂₉H₄₃N₂O₅Si (M+H)⁺ 527.29358, found 527.29360.

tert-Butyl(2R,4S)-4-(((tert-butyldiphenylsilyl)oxy)methyl)-2-(4-octylbenzoyl)pyrrolidine-1-carboxylate (22c): Prepared by applying in sequence general procedures C and B, starting from 22b (85 mg, 0.16 mmol). The crude was purified by flash column chromatography (EtOAc/hexane 1:1, Rf 0.16) to give 22c as a colorless oil (32 mg, 48% over two steps). α²⁵_{D}⁺ 16.6 (c 0.65, CHCl3). IR (neat), vₘₐₓ: 3434, 2924, 2854, 1687, 1605, 1394, 1365, 1223, 1161, 1129, 998, 882, 769 cm⁻¹. ¹H NMR (CDCl₃, 500 MHz, mixture of rotamers), δ: 7.89 (d, J=8.1 Hz, 0.8H), 7.85 (d, J=8.1 Hz, 1.2H), 7.27 (d, J=8.1 Hz, 1.2H), 7.24 (d, J=8.1 Hz, 0.8H), 5.37 (dd, J=9.4, 2.4 Hz, 0.4H), 5.24 (dd, J=9.3, 3.6 Hz, 0.6H), 3.82-3.75 (m, 1H), 3.66-3.57 (m, 2H), 3.33 (dd, J=10.7, 6.9 Hz, 0.6H), 3.27 (dd, J=10.6, 7.8 Hz, 0.4H), 2.67-2.62 (m, 2H), 2.57-2.46 (m, 1H), 2.20 (dt, J=12.9, 9.0 Hz, 0.6H), 2.12 (dt, J=12.2, 9.6 Hz, 0.4H), 2.03-1.99 (m, 1H), 1.93 (br. s, 0.4H), 1.76 (br. s, 0.6H), 1.66-1.58 (m, 2H), 1.45 (s, 3.6 H), 1.30-1.27 (m, 10H), 1.26 (s, 5.4H), 0.87 (t, J=6.9 Hz, 3H) ppm. ¹³C NMR (CDCl3, 125 MHz, mixture of rotamers), δ: 198.3, 197.8, 154.5, 153.9, 149.1, 132.6, 132.4, 128.7, 128.6, 128.3, 79.9, 79.8, 64.1, 64.0, 61.0, 60.9, 49.3, 48.9, 39.7, 38.8, 36.0, 33.1, 32.2, 31.8, 31.1, 31.0, 29.4, 29.2, 28.5, 28,2, 22.6, 14.1 ppm. HRMS (ESI) calcd. for C₂₅H₃₉NO₄Na (M+Na)⁺ 440.27713, found 440.27771.

((2R,4S)-4-(Hydroxymethyl)pyrrolidin-2-yl)(4-octylphenyl)methanone hydrochloride (22): Prepared according to general procedure A, starting from 22c (6 mg, 0.014 mmol). The crude was triturated in Et₂O to give product 22 as a white solid (5 mg, 100%). For biological testing a portion of the product was dissolved in the minimum amount of HPLC grade water, filtered (pore size=0.45 pm) and lyophilized. α²⁵_{D}⁺ 46.4 (c 0.25, CHCl₃). IR (neat), vₘₐₓ: 3370, 2922, 2852, 1683, 1605, 1570, 1464, 1416, 1400, 1373, 1350, 1310, 1263, 1182, 1164, 1092, 1060, 1013, 989, 967, 901, 722, 528 cm⁻¹.¹H NMR (CD₃OD, 500 MHz), δ: 8.01 (d, J=8.3 Hz, 2H), 7.45 (d, J=8.3 Hz, 2H), 5.43 (t, J=7.9 Hz, 1H), 3.70 (dd, J=10.9, 4.7 Hz, 1H), 3.65 (dd, J=10.9, 5.0 Hz, 1H), 3.62 (dd, J=11.2, 6.8 Hz, 1H), 3.34-3.30 (m, 1H), 2.77-2.73 (m, 2H), 2.60-2.52 (m, 2H), 2.19-2.13 (m, 1H), 1.71-1.65 (m, 2H), 1.37-1.31 (m, 10H), 0.91 (t, J=7.0 Hz, 3H) ppm. ¹³C NMR (CD₃OD, 125 MHz), δ: 193.1, 151.1, 130.2, 129.0, 63.2, 61.7, 48.0, 39.5, 35.6, 32.3, 31.6, 30.8, 29.1, 29.0, 28.9, 22.3, 13.0 ppm. HRMS (ESI) calcd. for C₂₀H₃₂NO₂ (M)⁺ 318.24276, found 318.24265.

tert-Butyl(2S,4R)-4-hydroxy-2-(2-(4-octylphenyl)acetyl)pyrrolidine-1-carboxylate (23b): 23a1 was synthesized in accordance with the general procedure C (500 mg, 1.24 mmol). 23a1 was obtained as a yellow oil which was submitted to general procedure B without further purification. The resulting residue was purified by flash column chromatography (hexane/EtOAc 4:6, Rf 0.40) to give 23b as a yellow oil (303 mg, 59% over 2 steps). α²⁰_{D} −66.58 (c 1.55, CHCl3). IR (neat), vₘₐₓ: 3433, 2923, 1676, 1394, 1159 cm⁻¹. ¹H NMR (CDCl₃, 500 MHz, mixture of rotamers), δ: 7.14-7.09 (m, 4H), 4.61-4.54 (m, 1H), 4.35 (bs, 1H), 3.81 (s, 0.75 H), 3.74-3.67 (m, 1.25 H), 3.63-3.61 (m, 0.63 H), 3.53-3.44 (m, 1.37 H), 2.58-2.55 (t, J=7.7 Hz, 2H), 2.08-1.79 (m, 2H), 1.59-1.56 (m, 2H), 1.46 (s, 3.51H), 1.39 (s, 5.19H), 1.30-1.26 (m, 10H), 0.89-0.86 (t, J=7.0Hz, 3H) ppm. ¹³C NMR (CDCl₃, 125 MHz, mixture of rotamers), δ: 207.9, 207.2, 154.8, 154.2, 142.0, 141.7, 130.6, 130.3, 129.6, 129.5, 128.8, 128.7, 80.7, 80.3, 70.4, 69.5, 63.4, 62.7, 55.2, 55.2, 47.1, 46.1, 35.6, 31.9, 31.5, 29.5, 29.3, 29.3, 28.3, 22.7, 14.1 ppm. HRMS (ESI) calcd. for C₂₅H₄₀NO₃ (M+H)⁺ 318.24276, found 318.24270.

tert-Butyl(2R,4S)-4-hydroxy-2-(2-(4-octylphenyl)acetyl)pyrrolidine-1-carboxylate (24b): 24a was synthesized in accordance with the general procedure C (484 mg, 1.20 mmol). 24a1 was obtained as a yellow oil which was submitted to general procedure XX without further purification. The resulting residue was purified by flash column chromatography (hexane/EtOAc 4:6, Rf 0.40) to give 24b as a yellow oil (340 mg, 68% over 2 steps). α²⁰_{D}⁺ 65.30 (c 0.19, CHCl₃). The spectral datas matched those reported for its enantiomer.

(2S,4R)-4-Hydroxy-2-(2-(4-octylphenyl)acetyl)pyrrolidin-1-ium chloride (23): 23 was synthesized in accordance with the general procedure A (25 mg, 0.06 mmol). 23 was obtained as a white powder (18 mg, 86%). α²⁰_{D}⁺ 91.9 (c 0.09, CHCl₃). IR (neat), vₘₐₓ: 3364, 3191, 2953, 2703, 1716, 1332, 1071, 763, 682 cm⁻¹. ¹H NMR (CDCl₃, 500 MHz), δ: 7.19-7.16 (s, 4H), 4.79-4.75 (dd, J=7.5Hz, 1H), 4.56 (m, 1H), 3.92 (s, 2H), 3.32-3.26 (m, 2H), 2.61 (dd, J=10.8, 7.8 Hz, 2H), 2.49-2.45 (dd, J=13.4, 7.7 Hz, 1H), 2.06-2.00 (ddd, J=13.5, 11.1, 4.0 Hz, 1H), 1.61-1.59 (m, 2H), 1.32-1.28 (m, 10H), 0.93-0.88 (t, J=6.98 Hz, 3H) ppm. ¹³C NMR (CDCl₃, 125 MHz), δ: 202.3, 201.9, 145.0, 142.0, 129.6, 129.4, 128.5, 128.4, 128.2, 69.6, 68.7, 64.6, 64.2, 53.4, 63.1, 35.1, 31.6, 31.3, 29.2, 29.0, 28.9, 22.3, 13.0 ppm. HRMS (ESI) calcd. for C₂₅H₄₀NO₃ (M+H)⁺ 318.24276, found 318.24750.

(2R,4S)-4-Hydroxy-2-(2-(4-octylphenyl)acetyl)pyrrolidin-1-ium chloride (24): 24 was synthesized in accordance with the general procedure A (17 mg, 0.04 mmol). 24 was obtained as a white powder (15 mg, 98%). α²⁰_{D} −80.04 (c 0.04, CHCl₃). The spectral datas matched those reported for its enantiomer.

tert-Butyl (2S,4R)-4-hydroxy-2-(1-hydroxy-2-(4-octylphenyl)ethyl)pyrrolidine-1-carboxylate (23b1): NaBH₄ (2.2 mg, 0.058 mmol, 1.2 eq.) was added in one portion to a solution of XX (20.0 mg, 0.050 mmol, 1.0 eq.) in dry MeOH (0.8 mL). The solution was stirred at rt for 2 h. Saturated aqueous NH₄Cl solution was added and the resulting aqueous layer was extracted with EtOAc (1×2 mL). The organic layers were combined, washed with brine (1×2 mL), dried over Na₂SO_{4,} filtered, concentrated. The resulting residue was purified by flash column chromatography (hexane/EtOAc 4:6, Rf:0.38) to give 23b1 as a colorless oil (15 mg, 75%). α²⁰_{D} −11.76 (c 1.02, CHCl₃). IR (neat), vₘₐₓ: 3409, 2923, 1664, 1403, 1160, 990, 771 cm⁻¹. ¹H NMR (CDCl₃, 500 MHz, mixture of rotamers), δ: 7.17-7.15 (d, J=7.8 Hz, 2H), 7.11-7.09 (d, J=7.9 Hz, 2H), 4.40 (bs, 1H), 4.11-4.09 (m, 1H), 3.83 (bs, 1H), 3.65 (bs, 1H), 3.39-3.36 (dd, J=12.1, 4.2 Hz, 1H), 2.82-2.78 (m , 1H), 2.57-2.54 (m, 3H), 2.09-2.06 (m, 1H), 1.89-1.77 (m, 2H), 1.65 (bs, 1H), 1.60-1.55 (m, 2H), 1.46 (s, 9H), 1.31-1.25 (m, 10H), 0.89-0.86 (t, J=7.0 Hz, 3H), ppm. ¹³C NMR (CDCl₃, 125 MHz, mixture of rotamers), δ: 158.1, 157.9, 141.1, 135.6, 129.5, 128.6, 80.9, 80.3, 73.0, 70.0, 55.6, 35.7, 32.0, 31.7, 29.6, 29.5, 29.4, 28.6, 28.5, 22.8, 14.2 ppm. HRMS (ESI) calcd. for C₂₅H₄₀NO₃ (M+H)⁺ 442.29278, found 442.29415.

tert-Butyl (2R,4S)-4-hydroxy-2-(1-hydroxy-2-(4-octylphenyl)ethyl)pyrrolidine-1-carboxylate (24b1): 24b1 was synthesized in accordance with the procedure of its enantiomer (14 mg, 0.03 mmol). The resulting residue was purified by flash column chromatography (hexane/EtOAc 4:6, Rf:0.38) to give 24b1 as a colorless oil (10 mg, 71%) . The spectral datas matched those reported for its enantiomer.

(2S,4R)-4-Hydroxy-2-(1-hydroxy-2-(4-octylphenyl)ethyl)pyrrolidin-1-ium chloride (23): 23 was synthesized in accordance with the general procedure A (6 mg, 0.014 mmol). 23 was obtained as a white powder (5 mg, 98%). IR (neat), vₘₐₓ: 3363, 2955, 1315, 968, 557 cm⁻¹. ¹H NMR (CDCl₃, 500 MHz, mixture of diastereomers), δ: 7.20-7.19 (d, J=8.1 Hz, 2H), 7.15-7.13 (d, J=8.1 Hz, 2H), 4.55-4.53 (m, 1H), 3.92-3.88 (m, 1H), 3.79-3.74 (m, 1H), 3.30-3.29 (m, 2H), 3.22-3.19 (m, 1H), 2.83-2.80 (m, 2H), 2.60-2.57 (t, J=, 2H), 2.08-2.04 (m, 1H), 1.97-1.91 (m, 1H), 1.61-1.58 (m, 2H), 1.33-1.29 (m, 10H), 0.91-0.89 (t, J=7.0 Hz, 3H) ppm. ¹³C NMR (CDCl₃, 125 MHz, mixture of diastereomers), δ: 142.7, 135.8, 130.7, 129.8, 72.8, 71.2, 63.9, 54.4, 42.2, 38.1, 36.7, 33.2, 33.0, 30.8, 30.6, 30.5, 23.9, 14.6 ppm. HRMS (ESI) calcd. for C₂₅H₄₀NO₃ (M+H)⁺ 320.25835, found 320.25841.

(2R,4S)-4-Hydroxy-2-(1-hydroxy-2-(4-octylphenyl)ethyl)pyrrolidin-1-ium chloride (24): 24 was synthesized in accordance with the general procedure A (10 mg, 0.024 mmol). 24 was obtained as a white powder (8 mg, 95%). The spectral datas matched those reported for its enantiomer.

tert-Butyl(2S,4R)-4-((di-tert-butoxyphosphoryl)oxy)-2-(4-octylbenzoyl)pyrrolidine-1-carboxylate (20c1): Di-tert-butyl diethylphosphoramidite (93%, 44 μL, 0.15 mmol, 2.0 eq.) and tetrazole (0.45 M in ACN, 0.22 mmol, 3.0 eq.) were added dropwise to a solution of 20c (30.0 mg, 0.074 mmol, 1.0 eq.) in dry THF (1 mL) at 0°. The resulting mixture was stirred for 1.5 hours, allowing it to warm up to room temperature. The reaction was cooled back to −30° C. whereby tBuOOH (5.0 M, 0.30 mmol, 4.0 eq.) was added dropwise. The resulting mixture was stirred at −30° C. for 15 minutes and at rt for 15 additional minutes. Afterwards, the reaction was cooled back to 0° C. whereby an aqueous NaHSO₃ solution (10% w/w, 2 mL) was added dropwise. The aqueous layer was extracted with EtOAc (3×2 mL). The resulting organic layer was washed with brine (2 mL), dried over Na₂SO₄, filtered and concentrated. The residue was purified by flash column chromatography (EtOAc/hexane 5:5+0.5% Pyridine, Rf:0.32) to give 20c1 as a colorless oil (26 mg, 62%). α²⁰_{D} −11.76 (c 1.02, CHCl₃). IR (neat), vₘₐₓ: 2977, 2926, 2855, 1701, 1396, 1260, 987, 753 cm⁻¹. ¹H NMR (CDCl₃, 500 MHz, mixture of rotamers), δ: 7.91-7.90 (d, J=8.2 Hz, 0.85 H), 7.87-7.86 (d, J=8.2 Hz, 1.15 H), 7.28-7.26 (d, J=8.7 Hz, 1.15 H), 7.25-7.24 (d, J=8.7 Hz, 0.85 H), 5.47-5.43 (t, J=8.0 Hz, 0.4 H), 5.37-5.33 (t, J=8.2 Hz, 0.6 H), 4.91 (m, 1H), 3.93-3.90 (dd, J=12.2 Hz, 0.6 H), 3.87-3.85 (m, 0.4 H), 3.75-3.69 (m, 1H), 2.67-2.62 (m, 2H), 2.62-2.54 (m, 1H), 2.09-2.01 (m, 1H), 1.62 (m, 2H), 1.50-1.48 (m, 18 H), 1.44 (s, 4H), 1.30-1.22 (m, 15 H), 0.87 (t, J=7.4 Hz, 3H) ppm. ¹³C NMR (CDCl₃, 125 MHz, mixture of rotamers), δ: 198.5, 198.0, 154.3, 153.7, 149.5, 149.4, 133.1, 133.0, 128.9, 128.9, 128.8, 128.5, 83.1, 83.0, 83.0 83.0, 80.4, 80.2, 75.8, 75.8, 75.2, 75.2, 59.5, 59.2, 53.7, 53.7, 53.4, 53.3, 36.2, 32.0, 31.2, 30.1, 30.0, 29.5, 59.3, 28.5, 28.2, 22.8, 14.2 ppm. HRMS (ESI) calcd. for C₂₅H₄₀NO₃ (M+H)⁺ 618.35301, found 618.35381.

tert-Butyl(2R, 4S)-4-((di-tert-butoxyphosphoryl)oxy)-2-(4-octylbenzoyl)pyrrolidine-1-carboxylate (21c1): 21c1 was synthesized in accordance with the procedure of its enantiomer (50 mg, 0.12 mmol). The residue was purified by flash column chromatography (EtOAc/hexane 5:5+0.5% Pyridine, Rf: 0.32) to give 21c1 as a colorless oil (71 mg, 63%). α²⁰_{D}⁺ 11.04 (c 0.53, CHCl₃). The spectral datas matched those reported for its enantiomer.

(2S,4R)-2-(4-Octylbenzoyl)-4-(phosphonooxy)pyrrolidin-1-ium chloride (29): 29 was synthesized in accordance with the general procedure A (25 mg, 0.04 mmol). 29 was obtained as a white solid (12 mg, 66%). α²⁰_{D} −29.39 (c 0.37, CHCl3). IR (neat), vₘₐₓ: 2923, 1685, 1165, 1032, 922, 513 cm⁻¹. ¹H NMR (CDCl3, 500 MHz, mixture of rotamers), δ: 8.01-7.99 (d, J=7.4 Hz, 2H), 7.42-7.40 (d, J=7.8 Hz, 2H), 5.53 (bs, 1H), 4.98 (bs, 1H), 3.70 (bs, 1H), 3.44 (bs, 1H), 2.99 (bs, 1H), 2.73-2.70 (t, J=7.6, 2 H), 2.08 (m, 1 H), 1.67-1.64 (m, 2H), 1.34-1.25 (m, 10H), 0.90-0.87 (t, J=7.0 Hz, 3H) ppm. ¹³C NMR (CDCl₃, 125 MHz, mixture of rotamers), δ: 193.0, 151.2, 130.3, 129.1, 129.0, 74.2, 61.9, 52.7, 38.0, 35.6, 31.6, 30.8, 29.1, 29.0, 28.9, 22.3, 13.0 ppm. HRMS (ESI) calcd. for C₂₅H₄₀NO₃ (M+H)⁺ 384.19344, found 384.19257.

(2S,4R)-2-(4-Octylbenzoyl)-4-(phosphonooxy)pyrrolidin-1-ium chloride (30): 30 was synthesized in accordance with the general procedure A (27 mg, 0.05 mmol). 30 was obtained as a white solid (14 mg, 78%). α²⁰_{D} +27.27 (c 0.55, CHCl₃). The spectral datas matched those reported for its enantiomer.

tert-Butyl(2S, 4R)-4-((di-tert-butoxyphosphoryl)oxy)-2-(2-(4-octylphenyl)acetyl)pyrrolidine-1-carboxylate (23b1): 23b1 was synthesized in accordance with the procedure from 20c1 (68 mg, 0.16 mmol). The resulting residue was purified by flash column chromatography (hexane/EtOAc 6:4, Rf: 0.37) to give 23b1 as a colorless oil (65 mg, 67%). α²⁰_{D} −45.23 (c 0.65, CHCl3). IR (neat), vₘₐₓ: 2925, 1696, 1393, 1262, 1160, 989 cm⁻¹. ¹H NMR (CDCl₃, 500 MHz, mixture of rotamers), δ: 7.14-7.08 (m, 4H), 4.78 (bs, 1H), 4.60-4.57 (t, J=8.9, 7.4 Hz, 0.47H), 4.54-4.50 (t, J=9.1, 7.7 Hz, 0.53H), 3.91-3.67 (m, 3H), 3.54-3.47 (ddd, J=16.6, 13.4, 3.3 Hz, 1H), 2.58-2.54 (m, 2H), 2.30-2.25 (m, 0.53H), 2.13-2.09 (m, 0.47H), 1.84-1.81 (m, 1H), 1.58-1.56 (m, 2H), 1.58-1.40 (m, 25H), 1.29-1.25 (m, 13H), 0.89-0.86 (t, J=7.0 Hz, 3H) ppm. ¹³C NMR (CDCl₃, 125 MHz, mixture of rotamers), δ: 207.1, 207.1, 154.6, 153.8, 142.2, 141.9, 130.6, 130.3, 129.7, 129.6, 129.0, 128.9, 83.1, 83.1, 83.1, 83.0, 82.9, 80.9, 80.5, 75.7, 75.6, 74.8, 74.8, 63.4, 62.5, 53.8, 53.7, 53.5, 53.5, 47.6, 46.3, 32.0, 30.0, 30.0, 29.6, 29.4, 28.5, 28.4, 22.8, 14.2 ppm. HRMS (ESI) calcd. for C₂₅H₄₀NO₃ (M+H)⁺ 610.38672, found 610.38470.

tert-Butyl (2R,4S)-4-((di-tert-butoxyphosphoryl)oxy)-2-(2-(4-octylphenyl)acetyl)pyrrolidine-1-carboxylate (24b1): 24b1 was synthesized in accordance with the procedure from 20c1 (68 mg, 0.16 mmol). The resulting residue was purified by flash column chromatography (hexane/EtOAc 6:4, Rf: 0.37) to give 24b1 as a colorless oil (66 mg, 68%). α²⁰_{D} +53.18 (c 0.85, CHCl₃). The spectral datas matched those reported for its enantiomer.

(2S,4R)-2-(2-(4-Octylphenyl)acetyl)-4-(phosphonooxy)pyrrolidin-1-ium chloride (31): 31 was synthesized in accordance with the general procedure A (30 mg, 0.05 mmol). 31 was obtained as a purple paste (12 mg, 57%). α²⁰_{D}+4.72 (c 1.35, CHCl₃). IR (neat), vₘₐₓ: 2922, 1724, 1514, 1173, 1009 cm⁻¹. ¹H NMR (CDCl3, 500 MHz), δ: 7.17 (m, 4H), 5.00 (bs, 1H), 4.82-4.77 (m, 1H), 3.98-3.92 (s, 2H), 3.60-3.57 (d, J=12.7 Hz, 1H), 3.42-3.39 (d, J=9.4 Hz, 1H), 2.83-2.78 (m, 1H), 2.62-2.58 (m, 2H), 2.18-2.13 (m, 1H), 1.60 (m, 2H), 1.33-1.29 (m, 10H), 0.92-0.88 (t, J=10.7 Hz, 3H) ppm. ¹³C NMR (CDCl₃, 125 MHz), δ: 201.7, 142.0, 141.6, 129.5, 129.5, 129.4, 129.4, 128.5, 128.4, 128.2, 75.0, 74.4, 74.4, 64.5, 64.1, 63.8, 63.3, 52.4, 52.3, 45.1, 35.1, 31.6, 31.3, 29.2, 29.0, 28.9, 22.3, 13.0 ppm. HRMS (ESI) calcd. for C₂₅H₄₀NO₃ (M+H)⁺ 398.20909, found 398.20750.

(2R,4S)-2-(2-(4-Octylphenyl)acetyl)-4-(phosphonooxy)pyrrolidin-1-ium chloride (32): 32 was synthesized in accordance with the general procedure A (35 mg, 0.06 mmol). 32 was obtained as a purple paste (15 mg, 60%). α²⁰_{D} −5.03 (c 1.20, CHCl₃). The spectral datas matched those reported for its enantiomer.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. A compound of formula 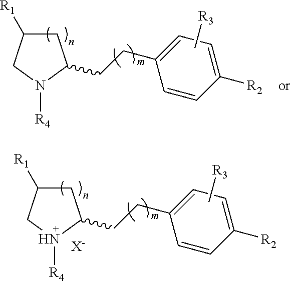
wherein:
R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, cyanide (CN), or a combination thereof;
R₄ is a functional group selected from H, alkyl including methyl (Me), tert-butyloxycarbonyl, or acyl;
X⁻ is an anion of the suitable acid;
n is an independently selected integer selected from 1, 2, or 3;
m is an independently selected integer selected from 0, 1 or 2; and comprising
wherein the linking group connecting the phenyl ring to the azacycle may optionally include one or more functional groups selected from the following:
a polar group in the alpha, beta or gamma position with regard to the azacycle selected from carbonyls (C═O), alcohols (CHOH), and alkoxys; and
a cyclic carbon chain extending from the alpha, beta or gamma positions with regard to the azacycle back to the N of the azacycle;
and a combination thereof.

2. The compound of claim 1, wherein the compound is selected from the group consisting of:

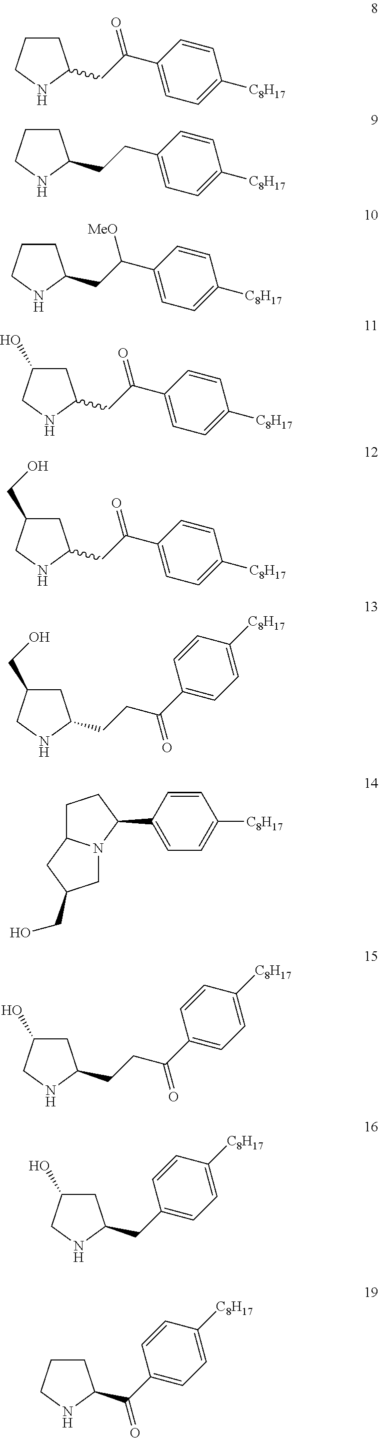

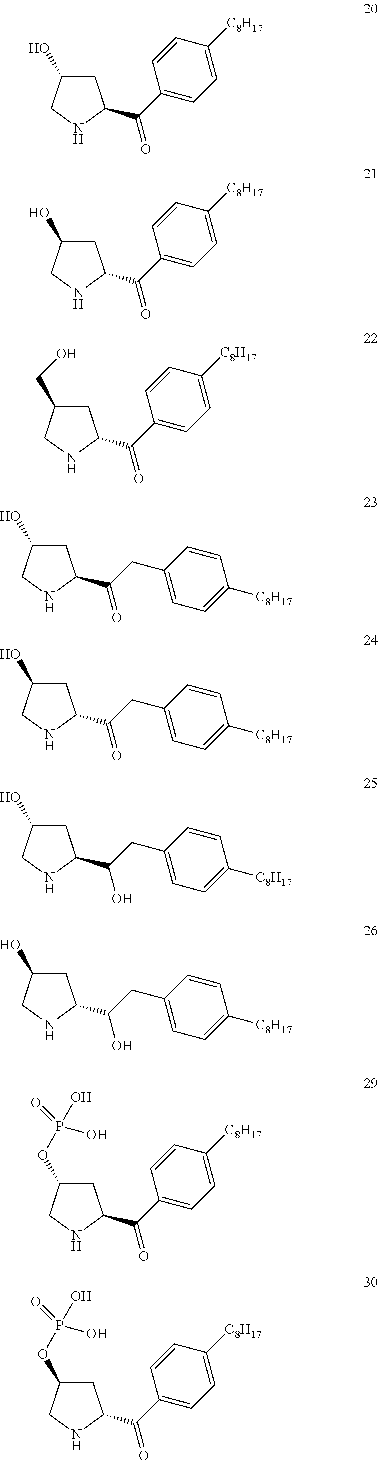

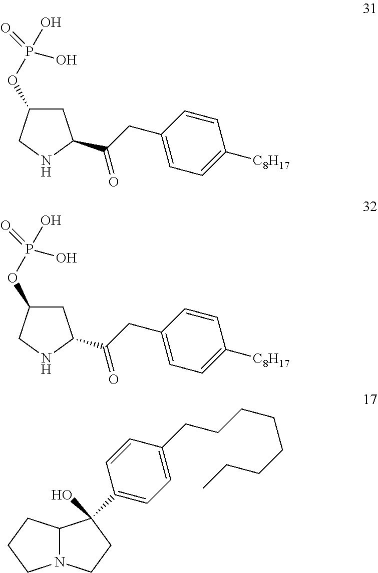

3. (canceled)

4. (canceled)

5. (canceled)

6. The compound of claim 1, wherein the compound is capable of having a cytotoxic or cytostatic effect on human neoplastic cells, and wherein the cytotoxic effect is defined by a reduction in the percentage of viable human neoplastic cells and the cytostatic effect is defined by reduction of proliferation of neoplastic cells.

7. The compound of claim 6, wherein the cytotoxic or cytostatic effect is achieved with a local 50% inhibitory concentration (IC₅₀) of less than twenty micromolar, wherein the local IC₅₀ is defined by the concentration of the compound that reduces the percentage of viable human neoplastic cells by 50%.

8. The compound of claim 6, wherein the human neoplastic cells are at least one of the following:
derived from at least one neoplasm, and wherein the at least one neoplasm is selected from the group consisting of: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors; and
characterized by one of: fast-growing, aggressive, Warburg-phenotypic, malignant, Ras-positive, PTEN-negative, having PI 3-kinase mutations, benign, metastatic, or nodular.

9. (canceled)

10. The compound of claim 1, wherein the compound is at least one of the following:
capable of exerting bioenergetic stress on human cells, wherein the bioenergetic stress is characterized by a decrease of at least one nutrient available to the human cells, and wherein the at least one nutrient is selected from one or more of the group: glucose, amino acids, nucleotides, and lipids; and
capable of inhibiting growth of a tumor comprised of human neoplastic cells, wherein growth is defined by at least one growth assessment, and wherein the at least one growth assessment is selected from the group consisting of: an increase in tumor diameter, an increase in tumor bioluminescence, an increase in tumor volume, an increase in tumor mass, or neoplastic cell proliferation.

11. The compound of claim 10, wherein the human cells are comprised of neoplastic and non-neoplastic cells, and wherein the bioenergetic stress results in greater percentage of cell death in the neoplastic cells relative to non-neoplastic cells.

12. (canceled)

13. A medicament for the treatment of a human disorder comprising:
a pharmaceutical formulation containing a therapeutically effective amount of one or more small molecule compounds having the formula 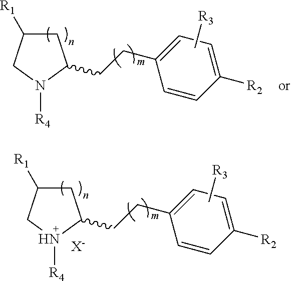
wherein:
R₁ is a functional group selected from H, an alkyl chain, OH, (CH₂)ₙOH, CHOH-alkyl, CHOH-alkyne, (CH₂)ₙOR', (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, cyanide (CN), or a combination thereof;
R₄ is a functional group selected from H, alkyl including methyl (Me), tert-butyloxycarbonyl, or acyl;
X⁻ is an anion of the suitable acid;
n is an independently selected integer selected from 1, 2, or 3;
m is an independently selected integer selected from 0, 1 or 2; and comprising
wherein the linking group connecting the phenyl ring to the azacycle may
optionally include one or more functional groups selected from the following:
a polar group in the alpha, beta or gamma position with regard to the azacycle selected from carbonyls (C═O), alcohols (CHOH), and alkoxys; and
a cyclic carbon chain extending from the alpha, beta or gamma positions with regard to the azacycle back to the N of the azacycle,
or a combination thereof.

14. The medicament of claim 13, wherein the one or more compounds is selected from the group consisting of:

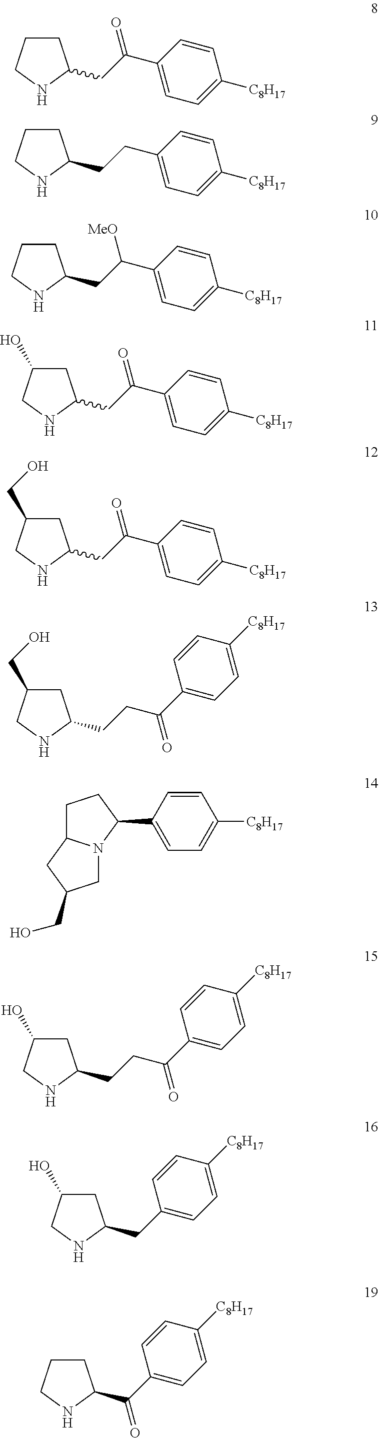

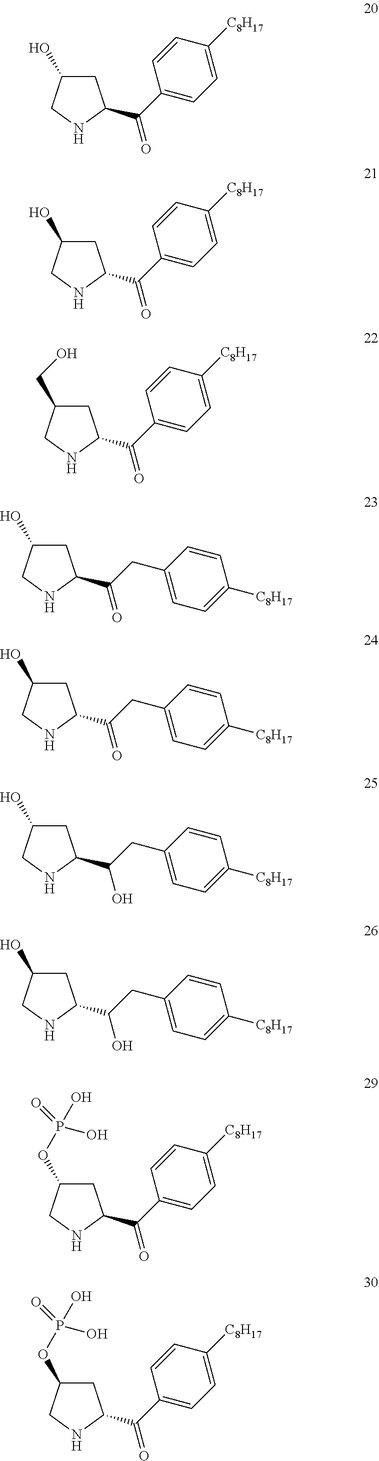

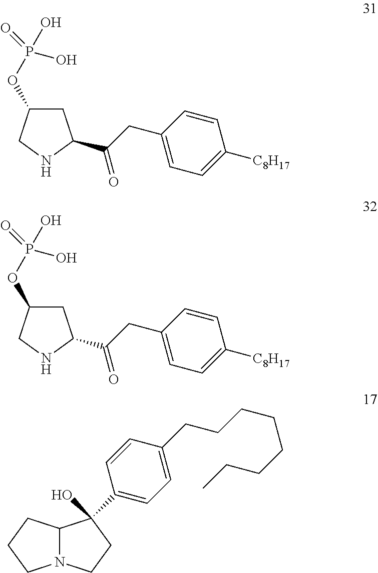

15. (canceled)

16. (canceled)

17. (canceled)

18. The medicament of claim 13, wherein the human disorder is at least one neoplasm, and wherein the at least one neoplasm is selected the group consisting of: acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors.

19. The medicament of claim 13, wherein the one or more compounds is at least one of the following:
capable of having a cytotoxic or cytostatic effect on human neoplastic cells, and wherein the cytotoxic effect is defined by a reduction in the percentage of viable human neoplastic cells and the cytostatic effect is defined by reduction of proliferation of neoplastic cells; and
capable of exerting bioenergetic stress on human cells, wherein the bioenergetic stress is characterized by a decrease of at least one nutrient available to the human cells, and wherein the at least one nutrient is selected from the group consisting of: glucose, amino acids, nucleotides, and lipids.

20. The medicament of claim 19, wherein the cytotoxic or cytostatic effect is achieved with a local 50% inhibitory concentration (IC₅₀) of less than twenty micromolar, wherein the local IC₅₀ is defined by the concentration of the compound that reduces the percentage of viable human neoplastic cells by 50%.

21. The medicament of claim 13, wherein the medicament is for the treatment of a neoplasm characterized by at least one of: fast-growing, aggressive, Warburg-phenotypic, malignant, Ras-positive, PTEN-negative, having PI 3-kinase mutations, benign, metastatic, or nodular.

22. (canceled)

23. The medicament of claim 19, wherein the human cells are comprised of neoplastic and non-neoplastic cells, and wherein the bioenergetic stress results in greater percentage of cell death in the neoplastic cells relative to the non-neoplastic cells.

24. The medicament of claim 13, wherein the pharmaceutical formulation at least one of the following:
is capable of inhibiting growth of a tumor comprising human neoplastic cells, wherein growth is defined by at least one growth assessment, and wherein the at least one growth assessment is selected from one or more of the group: an increase in tumor diameter, an increase in tumor bioluminescence, an increase in tumor volume, an increase in tumor mass, and neoplastic cell proliferation; and
further comprises at least one cytotoxic FDA-approved compound for the treatment of a neoplasm.

25. (canceled)

26. The medicament of claim 23, wherein the at least one cytotoxic FDA-approved compound is selected from the group consisting of:
methotrexate, gemcitabine, tamoxifen, taxol, docetaxel, and enzalutamide.

27. A method of treatment of a human disorder comprising:
administering a pharmaceutical formulation to a human subject, the pharmaceutical formulation containing a therapeutically effective amount of one or more small molecule compounds having the formula 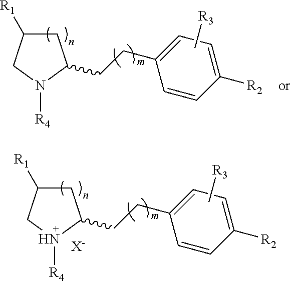
wherein:
R₁ is a functional group selected from H, an alkyl chain, OH, (CH₂)ₙOH, CHOH-alkyl, CHOH-alkyne, (CH₂)ₙOR', (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, cyanide (CN), or a combination thereof;
R₄ is a functional group selected from H, alkyl including methyl (Me), tert-butyloxycarbonyl, or acyl;
X⁻ is an anion of the suitable acid;
n is an independently selected integer selected from 1, 2, or 3;
m is an independently selected integer selected from 0, 1 or 2; and comprising
wherein the linking group connecting the phenyl ring to the azacycle may
optionally include one or more functional groups selected from the following:
a polar group in the alpha, beta or gamma position with regard to the azacycle selected from carbonyls (C═O), alcohols (CHOH) , and alkoxys; and
a cyclic carbon chain extending from the alpha, beta or gamma positions with regard to the azacycle back to the N of the azacycle,
or a combination thereof.

28. The method of claim 27, wherein the one or more compounds is selected from the group consisting of:

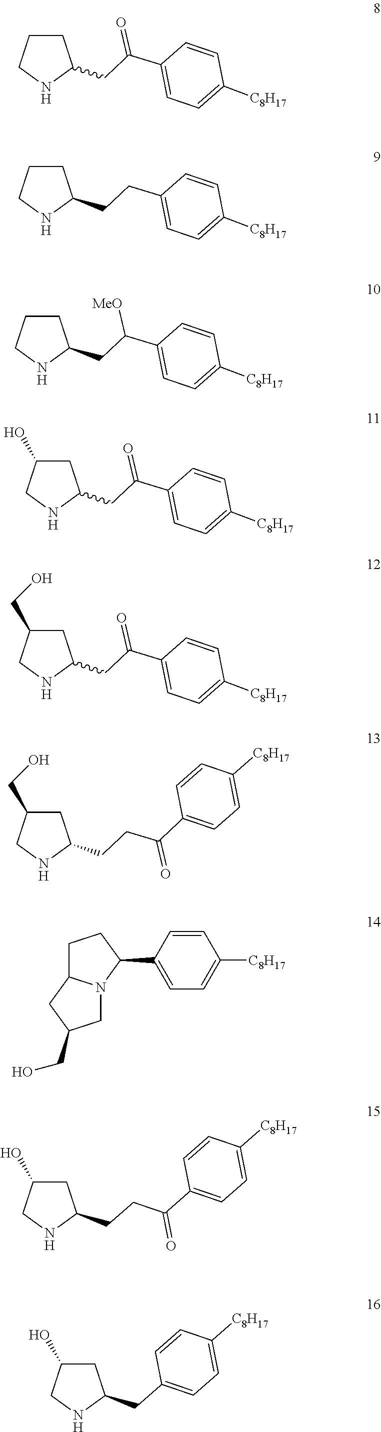

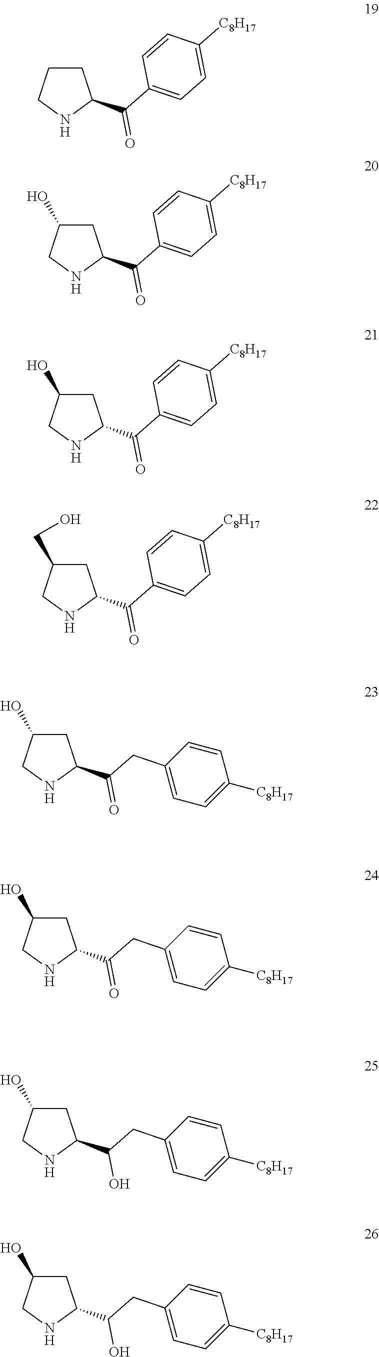

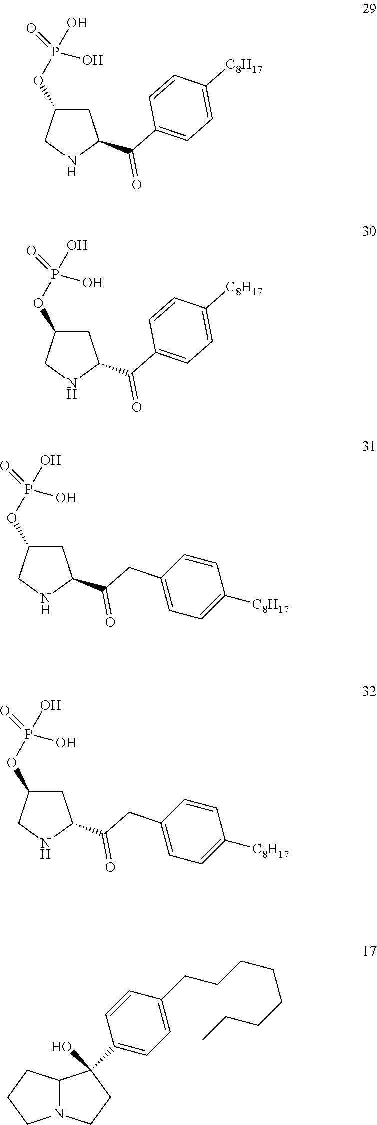

29. (canceled)

30. (canceled)

31. (canceled)

32. The method of claim 27, further comprising diagnosing the human subject with at least one human disorder.

33. The method of claim 32, wherein the at least one human disorder is at least one of the following:
a neoplasm, and wherein the neoplasm is selected from one or more of the group:
acute lymphoblastic leukemia (ALL), acute myeloid leukemia (AML), anal cancer, astrocytomas, basal cell carcinoma, bile duct cancer, bladder cancer, breast cancer, cervical cancer, chronic lymphocytic leukemia (CLL) chronic myelogenous leukemia (CML), chronic myeloproliferative neoplasms, colorectal cancer, endometrial cancer, ependymoma, esophageal cancer, esthesioneuroblastoma, Ewing sarcoma, fallopian tube cancer, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, vaginal cancer, and vascular tumors; and
characterized by at least one neoplasm characterization, and wherein the at least one neoplasm characterization is selected from one or more of the group: fast-growing, aggressive, Warburg-phenotypic, malignant, Ras-positive, PTEN-negative, having PI 3-kinase mutations, benign, metastatic, or nodular.

34. The method of treatment of claim 27, wherein the pharmaceutical formulation is at least one of the following:
inhibits growth of a tumor comprising human neoplastic cells, wherein growth is defined by at least one growth assessment, and wherein the at least one growth assessment is selected from one or more of the group: an increase in tumor diameter, an increase in tumor bioluminescence, an increase in tumor volume, an increase in tumor mass, and neoplastic cell proliferation; and
combined with at least one cytotoxic FDA-approved compound.

35. (canceled)

36. The method of treatment of claim 27, where in the treatment is combined with an FDA-approved standard of care.

37. (canceled)

38. The method of treatment of claim 34, wherein the at least one cytotoxic FDA-approved compound is selected from the group consisting of:
methotrexate, gemcitabine, tamoxifen, taxol, docetaxel, and enzalutamide.

39. A compound having the formula: 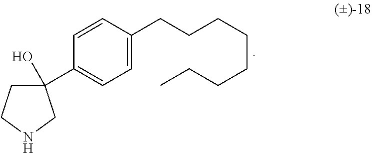 [SMILES: CCCCCCCCc1ccc(C2(O)CCNC2)cc1]
